(19) United States
(12) Patent Application Publication
(10) Pub. No.: US 2026/0049991 A1
(43) Pub. Date: Feb. 19, 2026

(54) SINGLE-MOLECULE PEPTIDE SEQUENCING USING GUANIDINYLATING AGENTS

(71) Applicant: Glyphic Biotechnologies, Inc., New York, NY (US)

(72) Inventors: Joshua Young Cynming YANG, Richardson, TX (US); Jai PRAKASH, Fremont, CA (US); Asmamaw WASSIE, Leander, TX (US); Daniel Masao ESTANDIAN, Antioch, CA (US)

(21) Appl. No.: 19/278,983

(22) Filed: Jul. 24, 2025

Related U.S. Application Data
(63) Continuation of application No. PCT/US24/13211, filed on Jan. 26, 2024.
(60) Provisional application No. 63/481,932, filed on Jan. 27, 2023.

(51) Int. Cl. G01N 33/68; G01N 33/487; G01N 33/543

(52) U.S. Cl. CPC G01N 33/6824; G01N 33/48721; G01N 33/543

(57) ABSTRACT

The present disclosure provides reagents and methods useful for single-molecule sequencing of proteins through use of a sequencing reagent of Formula I, IV, IV′, IV″, IV-A, IV-B, or V-C. The reagents and methods described herein provide for high-throughput single molecule and high efficiency protein and peptide sequencing in mild conditions allowing for high resolution investigation of complex biological systems.

### CROSS REFERENCE

This application is a continuation of International Patent Application No. PCT/US24/13211, filed Jan. 26, 2024, which claims benefit of U.S. Provisional Patent Application No. 63/481,932 filed Jan. 27, 2023, which is incorporated by reference herein in its entirety.

### STATEMENT REGARDING FEDERALLY SPONSORED RESEARCH OR DEVELOPMENT

This invention was made with government support under Grant Number HG012563, awarded by the National Institutes of Health. The government has certain rights in the invention.

### BACKGROUND

Proteins serve a critical role at a cellular level, carrying out a variety of integral functions. Having the technology required to quantify and identify proteins is crucial to understanding their contributions to biological function. Advancements in proteomics have lagged behind while DNA sequencing has rapidly advanced the study of genomics primarily due to technologies that allow for high-throughput sequencing. Current methodologies available for studying proteins include mass spectrometry, Edman sequencing, and immunohistochemistry.

Mass spectrometry (MS) has enabled protein identification and quantification based on the mass/charge ratio of peptide fragments, which can be bioinformatically mapped back to a genomic database. However, MS has yet to quantify a complete set of proteins from a biological system, despite significant advancements. MS exhibits attomole detection for whole proteins and subattomole sensitives after fractionation. Yet, functionally-important, low copy-number proteins that make up about 10% of mammalian protein expression remain undetected.

Edman degradation allows for sequential and selective removal of single N-terminal amino acids which are subsequently identified via HPLC (High-Performance Liquid Chromatography). Edman protein sequencing removes the first N-terminal amino acid for identification using phenyl isothiocyanate (PITC) to conjugate to the N-terminal amino acid, then upon acid and heat treatment, the PITC-labeled N-terminal amino acid is removed. Although Edman sequencing can have 98% efficiency, a major drawback is that it is inherently low throughput, requiring a single highly purified protein, and the inapplicability to systems-wide biology. Moreover, Edman degradation presents a number of other drawbacks including harsh reaction conditions such as heat and acidic conditions which are not amenable to using or analyzing nucleic acids, resultant chiral residues which can hinder detection via binding agents that are stereoisomer-specific, and modification of lysine residues, which can prevent further functionalization of the lysine side chains. Both Edman degradation and mass spectrometry can sequence proteins but lack single molecule sensitivity and do not provide spatial information of proteins in the context of cells.

In regards to spatial information, immunohistochemistry is a protein identification method that allows visualization of cellular localization of proteins but does not provide sequence information. Immunohistochemistry involves the identification of proteins via recognition with fluorophore-conjugated antibodies. This approach excludes protein sequence information but can identify proteins and their respective localizations. A major limitation is the scalability, since even the perfect construction of specific antibodies for every protein in the proteome would require around 25,000 antibodies and, ˜6250 rounds of four-color imaging.

### SUMMARY

Considering the present need for improved methods of single molecule protein sequencing, presented herein are formulas, compounds, and methods for addressing the abovementioned need. In some embodiments, sequencing reagents are provided herein comprising a reactive group, a capture-binding moiety, and a linker. In some instances, these sequencing reagents allow for sequencing of polymeric analytes, such as polypeptides. Provided herein are sequencing reagents comprising reactive groups comprising guanidinylating agents. Also provided herein are methods of sequencing polymeric analytes using the sequencing reagents provided herein. A brief summary of various exemplary embodiments is presented. Some simplifications and omissions may be made in the following summary, which is intended to highlight and introduce aspects of certain embodiments disclosed herein, but not to limit the scope of the disclosure. Detailed descriptions of various embodiments adequate to allow those of ordinary skill in the art to make and use the concepts disclosed herein will follow in later sections.

One aspect of the present disclosure provides a sequencing reagent of Formula I or a stereoisomer, tautomer, or salt thereof.

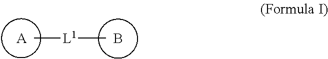

In some embodiments, A comprises a reactive group configured to form a covalent bond with an N-terminal amino acid of a peptide, wherein the reactive group comprises a guanidinylating agent. In some embodiments, B comprises a capture-binding moiety. In some embodiments, L¹ comprises a linker coupled to A and B.

In some embodiments, A comprises a compound of Formula (I-A):

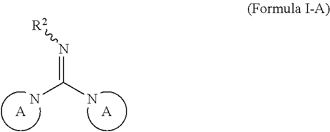

In some embodiments, R² is H, R⁴, OH, OR⁴, NH₂, or —NHR⁴. In some embodiments, R⁴ is C₁₋₆ alkyl, which is optionally substituted with one or two members selected from halo, alkyl, alkoxy, haloalkyl, phenyl, and a 5- or 6-membered heteroaryl, wherein the phenyl and 5- or 6-membered heteroaryl are optionally substituted with one or two members selected from halo, —OH, C₁₋₄ alkyl, C₁₋₄ alkoxy, C₁₋₄ haloalkyl, NO₂, CN, COOR″, and CON(R″)₂, where each R″ is independently H or C₁₋₃ alkyl.

In some embodiments, ring A is a 5-membered heteroaryl ring containing up to three N atoms as ring members and is optionally fused to an additional phenyl or a 5-6 membered heteroaryl ring, and wherein the 5-membered heteroaryl ring and phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R^{a}, —NR₂, phenyl, and 5-6 membered heteroaryl. In some embodiments, each R is independently selected from H and C₁₋₃ alkyl optionally substituted with OH, OR^{a}, —NH₂, —NHR^{a}, or —NR^{a}₂. In some embodiments, each R^{a} is C₁₋₃ alkyl, optionally substituted with OH, oxo, C₁₋₂ alkoxy, or CN. In some embodiments, two R, or two R″, or two R^{a} on the same N can optionally be taken together to form a 4-7 membered heterocyclic ring, optionally containing an additional heteroatom selected from N, O, and S as a ring member, and optionally substituted with one or two groups selected from halo, C₁₋₂ alkyl, OH, oxo, C₁₋₂ alkoxy, and CN.

In some embodiments, the reactive group comprises the structure of Formula I-B:

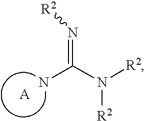

or a stereoisomer, tautomer, or salt thereof.

In some embodiments, R² are each independently hydrogen, R⁴, OH, OR⁴, NH₂, or —NHR⁴. In some embodiments, R⁴ is C₁₋₆ alkyl or C₁₋₆ alkoxy, each of which is optionally substituted with halo, C₁₋₆ alkyl, C₁₋₆ alkoxy, C₁₋₆ haloalkyl, oxo, aryl, and 5-6-membered heteroaryl, wherein the aryl and heteroaryl are optionally substituted with one or two members selected from halo, —OH, C₁₋₄ alkyl, C₁₋₄ alkoxy, C₁₋₄ haloalkyl, NO₂, CN, COOR″, and CON(R″)₂, where each R″ is independently H or C₁₋₃ alkyl. In some embodiments, ring A is a 5-6-membered heteroaryl ring comprising up to three N atoms as ring members and is optionally fused to an additional aryl or 5-6-membered heteroaryl ring, and wherein the aryl and 5-6-membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R^{a}, —NR₂, phenyl, and 5-6 membered heteroaryl. In some embodiments, each R is independently selected from hydrogen and C₁₋₃ alkyl optionally substituted with OH, OR^{a}, —NH₂, —NHR^{a}, or —NR^{a}₂. In some embodiments, each R^{a} is C₁₋₃ alkyl, optionally substituted with OH, oxo, C₁₋₂ alkoxy, or CN. In some embodiments, two R, or two R″, or two R^{a} on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, and CN.

In some embodiments, each R² are independently hydrogen or R⁴.

In some embodiments, R⁴ is C₁₋₆ alkyl or C₁₋₆ alkoxy, each optionally substituted with one or more of C₁₋₆ alkyl, oxo, and aryl.

In some embodiments, Ring A is pyrazole or triazole, each optionally fused with aryl.

In some embodiments, Ring A is pyrazole, triazole, or

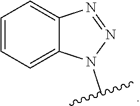 [SMILES: *n1nnc2ccccc21]

In some embodiments, the reactive group comprising the structure Formula I-B is:

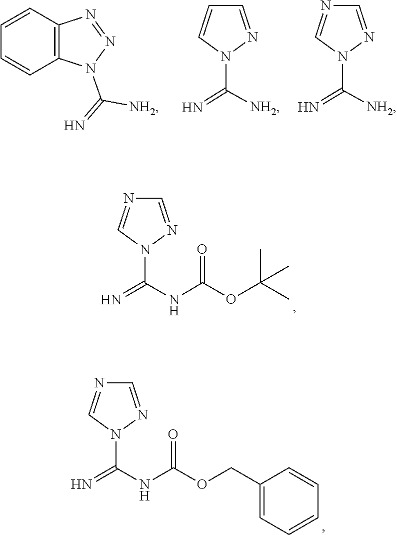 [SMILES: N=C(N)n1nnc2ccccc21 ; N=C(N)n1cccn1 ; N=C(N)n1cncn1 ; CC(C)(C)OC(=O)NC(=N)n1cncn1 ; N=C(NC(=O)OCc1ccccc1)n1cncn1]

stereoisomer, tautomer, or salt thereof.

In some embodiments, the capture-binding moiety comprises a polymer. In some embodiments, the polymer comprises polyethylene glycol (PEG). In some embodiments, PEG comprises between 1 and 20 monomers. In some embodiments, the polymer comprises deoxyribonucleic acid (DNA) or ribonucleic acid (RNA). In some embodiments, the polymer is covalently linked to a substrate.

In some embodiments, the capture-binding moiety is covalently linked to a surface bound linker. In some embodiments, the capture-binding moiety is covalently linked to the surface-bound linker, and wherein the surface-bound linker is linked to the surface.

In some embodiments, the capture-binding moiety comprises a click chemistry moiety. In some embodiments, the capture-binding moiety comprises an azide or an alkyne.

In some embodiments, the capture-binding moiety is configured to couple to a capture-moiety, wherein the capture moiety comprises a thiol group.

In some embodiments, the substrate comprises a thiol group.

In some embodiments, the capture-binding moiety comprises:

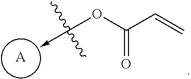

In some embodiments,

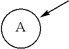

indicates orientation of the capture-binding moiety relative to the reactive group.

In some embodiments, capture-binding moiety comprises a thiol group, and wherein the capture-binding moiety is configured to couple to a surface that comprises an acrylate group.

In some embodiments, the reactive group comprises Formula II-A:

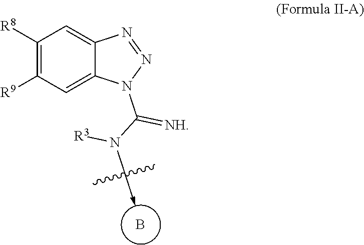

In some embodiments, R³ is hydrogen or substituted or unsubstituted C₁₋₆ alkyl, or heteroalkyl. In some embodiments, R⁸ and R⁹ are independently hydrogen or NO₂. In some embodiments,

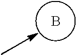

indicates orientation of the reactive group relative to the capture-binding moiety.

In some embodiments, the reactive group comprises Formula III-A:

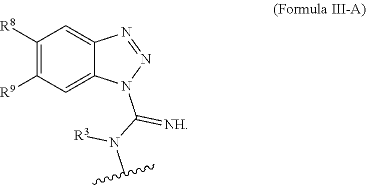

In some embodiments, R³ is hydrogen or substituted or unsubstituted C₁₋₆ alkyl. In some embodiments, R⁸ and R⁹ are independently hydrogen or NO₂.

In some embodiments, R³ comprises an alkane group, and wherein

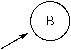

indicates orientation of the reactive group relative to the capture-binding moiety.

In some embodiments, L¹ comprises a cleavable linker. In some embodiments, the cleavable linker comprises a disulfide bond, a hydrazone, a PEG linker, a DNA molecule comprising a cleavage site, a peptide that is cleavable by an enzyme, an ester, or a de-click chemistry moiety.

In some embodiments, the reactive group is linked by a covalent bond with the N-terminal amino acid of the polypeptide. In some embodiments, the capture-binding moiety is linked directly or indirectly to a substrate. In some embodiments, the reactive group is linked by a covalent bond with the N-terminal amino acid of the polypeptide linked to the substrate; and wherein the capture-binding moiety is linked directly or indirectly to the substrate.

Another aspect of the present disclosure provides a sequencing reagent having a structure of Formula IV, or a stereoisomer, tautomer, or salt thereof:

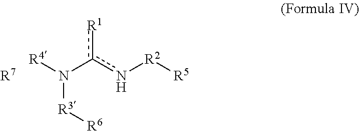

In some embodiments, R¹ comprises one or more leaving groups. In some embodiments, R² and R^{3′} are independently selected from one or more linkers, hydrogen, or null. In some embodiments, R² and R^{3′} are taken together with the atoms from which they are attached to form an aryl, heteroaryl, cycloalkyl, or heterocycloalkyl, the aryl, heteroaryl, cycloalkyl, or heterocycloalkyl being optionally substituted with one or more linker-click-chemistry moieties. In some embodiments, R^{4′} is one or more linkers, hydrogen, or null. In some embodiments, R⁵, R⁶, and R⁷ are independently click-chemistry moieties or null. In some embodiments, [symbol] is a single [symbol] or double bond. In some embodiments, when present, at least one of R⁵, R⁶, and R⁷ comprise a click-chemistry moiety.

In some embodiments, provided herein is a sequencing reagent having the structure of Formula IV′ or a stereoisomer, tautomer, or salt thereof:

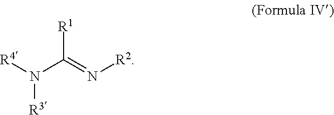

In some embodiments, R¹ comprises one or more leaving groups. In some embodiments, R² and R^{3′} are taken together with the atoms from which they are attached to form an aryl, heteroaryl, cycloalkyl, or heterocycloalkyl, the aryl, heteroaryl, cycloalkyl, or heteroalkyl being substituted with one or more linker-click chemistry moieties. In some embodiments, R^{4′} is hydrogen.

In some embodiments, R² and R^{3′} are taken together with the atoms from which they are attached to form heterocycloalkyl substituted with one or more linker-click chemistry moieties.

In some embodiments, provided herein is a sequencing reagent having the structure of Formula IV″, or a stereoisomer, tautomer, or salt thereof:

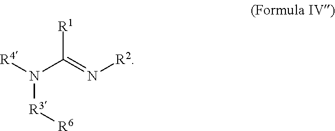

In some embodiments, R comprises one or more leaving groups. In some embodiments, R² and R^{3′} are independently selected from a linker, hydrogen, or null. In some embodiments, R^{4′} is substituted or unsubstituted C₁₋₆ alkyl, substituted or unsubstituted C₁₋₆ heteroalkyl, substituted or unsubstituted C₁₋₆ alkoxy, hydrogen, or null. In some embodiments, R⁶, if present, is a click chemistry moiety.

In some embodiments, the leaving group comprises an electrophilic group. In some embodiments, the electrophilic group comprises S, SCH₃, SO₃H, or NHTf.

In some embodiments, the electrophilic group comprises SR*, and R* comprises H, R′, OH, OR′, NH₂, or NHR′, wherein R′ is a C₁₋₆ alkyl optionally substituted with one or more members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂, where each R″ is independently H or C₁₋₃ alkyl.

In some embodiments, the electrophilic group is substituted or unsubstituted C₃₋₁₀ aryl or substituted or unsubstituted C₃₋₁₀ heteroaryl.

In some embodiments, one or more of R² and R⁵ are null.

In some embodiments, R is:

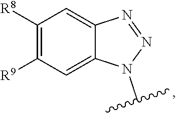

wherein R⁸ and R⁹ are independently selected from hydrogen or an electron withdrawing group.

In some embodiments, the sequencing reagent provided herein has a structure of Formula V-C or a stereoisomer, tautomer, or salt thereof:

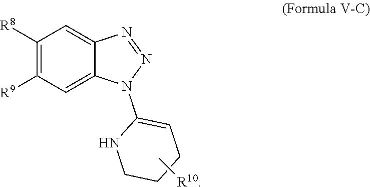

In some embodiments, R⁸ and R⁹ are independently selected from hydrogen or an electron withdrawing group. In some embodiments, R¹⁰ is a linker-click chemistry moiety (e.g., CH₂-azide).

In some embodiments, R^{3′} and R^{4′} are connected by a ring structure.

In some embodiments, the linker is a cleavable linker. In some embodiments, the cleavable linker comprises a disulfide bond, hydrazone, a PEG linker, a DNA molecule comprising a cleavage site, a peptide that is cleavable by an enzyme, an ester, or a de-click chemistry moiety.

In some embodiments, the click-chemistry moiety is azide or alkyne.

In some embodiments, R² and R⁵ are null.

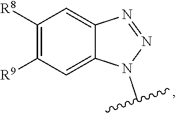

In some embodiments, R¹ is and wherein R⁸ and R⁹ are independently selected from hydrogen or an electron withdrawing group.

In some embodiments, the electron withdrawing group comprises a haloalkyl, a halogen, an amide, or a carbonyl. In some embodiments, the electron withdrawing group comprises a haloalkyl, a halogen, an amide, a carbonyl, or NO₂. In some embodiments, the electron withdrawing group comprises NO₂. In some embodiments, the electron withdrawing group comprises CF₃. In some embodiments, the electron withdrawing group comprises SO₃H. In some embodiments, the electron withdrawing group comprises NR₃, wherein R comprises an alkyl. In some embodiments, the electron withdrawing group comprises NH₃. In some embodiments, the electron withdrawing group comprises CN. In some embodiments, the electron withdrawing group comprises COCl.

In another aspect of the present disclosure, provided herein is a sequencing reagent has the structure of Formula IV-A, or a stereoisomer, tautomer, or salt thereof:

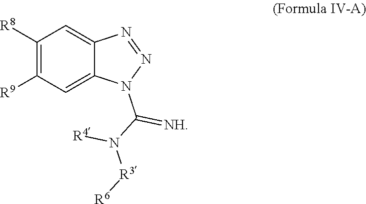

In some embodiments, R^{3′} comprises a linker. In some embodiments, R^{4′} is substituted or unsubstituted C₁₋₆ alkyl, substituted or unsubstituted C₁₋₆ heteroalkyl, substituted or unsubstituted C₁₋₆ alkoxy, hydrogen, or null. In some embodiments, R⁶, if present, is a click-chemistry moiety. In some embodiments, R⁸ and R⁹ are independently selected from hydrogen or an electron withdrawing group.

In some embodiments, R⁴′ is unsubstituted C₁₋₆ alkyl. In some embodiments, R^{4′} is C₁ alkyl.

In some embodiments, R^{3′} is C₁-C₆ alkyl.

In some embodiments, the electron withdrawing group comprises a halogen, a haloalkyl, an amide, or a carbonyl. In some embodiments, the electron withdrawing group comprises a haloalkyl, a halogen, an amide, a carbonyl, or NO₂. In some embodiments, the electron withdrawing group comprises NO₂. In some embodiments, the electron withdrawing group comprises CF₃. In some embodiments, the electron withdrawing group comprises SO₃H. In some embodiments, the electron withdrawing group comprises NR₃, wherein R comprises an alkyl. In some embodiments, the electron withdrawing group comprises NH₃. In some embodiments, the electron withdrawing group comprises CN. In some embodiments, the electron withdrawing group comprises COCl.

In some embodiments, at least one of R⁸ and R⁹ are NO₂. In some embodiments, R⁸ is NO₂. In some embodiments, R⁹ is NO₂.

In some embodiments, R⁶ comprises an azide. In some embodiments, R⁶ comprises an alkyne.

In some embodiments, R^{4′} is an alkyl. In some embodiments, R^{4′} is CH₂CH₂CH₃.

In some embodiments, R⁸ is hydrogen.

In some embodiments, R⁹ is NO₂.

In some embodiments, R^{3′} is an alkyl (e.g., C₁-C₆ alkyl).

In some embodiments, the sequencing reagent of Formula IV-A is:

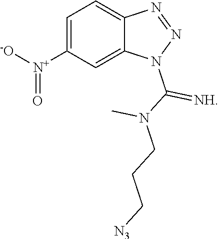

In some embodiments, R¹ is S.

Another aspect of the present disclosure provides a sequencing reagent, wherein the sequencing reagent represented by Formula IV is Formula TV-B:

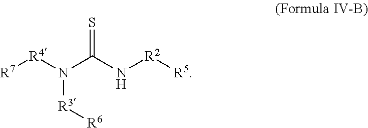

In some embodiments, R², R^{3′}, and R^{4′} are independently selected from hydrogen, one or more linkers, or null. In some embodiments, R⁵, R⁶, and R⁷ are independently click-chemistry moieties or null.

In some embodiments, the click-chemistry moiety comprises an azide or an alkyne. In some embodiments, the linker is a cleavable linker. In some embodiments, the cleavable linker comprises a disulfide bond, a hydrazone, a PEG linker, a DNA molecule comprising a cleavage site, a peptide that is cleavable by an enzyme, an ester, or a de-click chemistry moiety.

In some embodiments, R² is an alkyl. In some embodiments, R² is hydrogen.

In some embodiments, R^{3′} is C₁-C₆ alkyl.

In some embodiments, R⁶ is an azide.

In some embodiments, R^{4′} is hydrogen.

In some embodiments, R⁵ is an azide.

In some embodiments, R^{3′} and R⁵ are hydrogen.

In some embodiments, the sequencing reagent, wherein the sequencing reagent represented by Formula IV-B is:

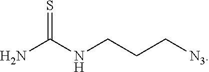

In some embodiments, the sequencing reagent is selected from:

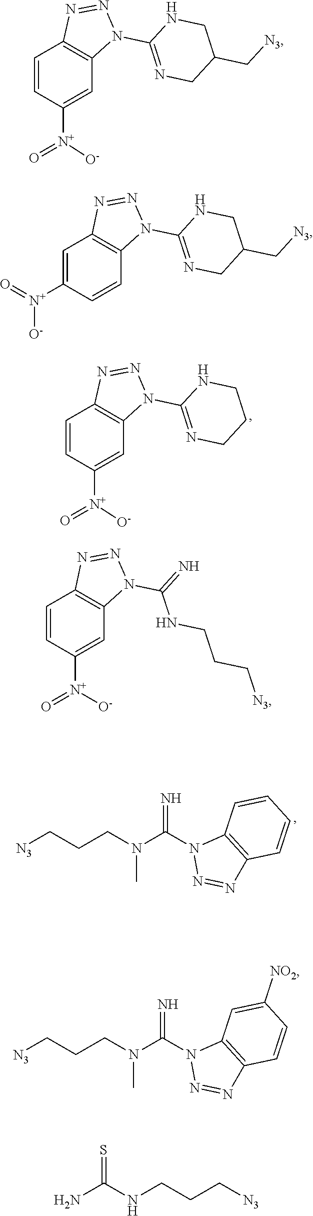

stereoisomer, tautomer, or salt thereof.

In some embodiments, the linker comprises PEG.

Another aspect of the present disclosure provides a method of using the sequencing agents provided herein. In some embodiments the method comprises (a) providing a substrate, a substrate-bound capture moiety, and a polymeric analyte, (b) contacting the polymeric analyte with the sequencing reagent, wherein the sequencing reagent binds to a monomer of the polymer analyte to form a sequencing reagent-monomer complex, (c) tethering the sequencing reagent-monomer complex to the substrate via the substrate-bound capture moiety, (d) cleaving the sequencing reagent-monomer complex from the polymeric analyte, thereby providing a detectable complex, and (e) detecting the detectable complex.

In some embodiments, the polymeric analyte comprises a polypeptide. In some embodiments, the monomer comprises a terminal amino acid residue. In some embodiments, the substrate-bound capture moiety comprises a DNA primer. In some embodiments, detecting the detectable complex comprises contacting the sequencing reagent-monomer complex with a binding agent. In some embodiments, the binding agent comprises an antibody, nanobody, single chain variable fragment (scFv), or aptamer. In some embodiments, the binding agent comprises a polymerizable molecule. In some embodiments, the polymerizable molecule comprises a nucleic acid molecule.

In some embodiments, the method further comprises coupling the nucleic acid molecule to the substrate-bound capture moiety or an additional substrate-bound capture moiety.

In some embodiments, the method further comprises repeating (b)-(e), thereby sequencing the polymeric analyte.

In some embodiments, in (a), the polymeric analyte is coupled to the substrate.

In some embodiments, (d) is performed chemically or enzymatically.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A schematically shows an example workflow for processing polymeric analytes molecules (e.g., peptides) described herein. FIG. 1B schematically shows another example workflow for processing polymeric analytes in solution. FIG. 1C schematically shows another example workflow for processing polymeric analytes and detection. FIG. 1D schematically shows another example workflow for processing polymeric analytes and detection using a nanopore sequencing system. FIG. 1E schematically shows another example workflow for processing polymeric analytes and detection using a nanopore sequencing system. FIG. iF schematically shows another example workflow for processing polymeric analytes and detection in solution.

FIG. 2 schematically shows an example linker for connecting polymerizable molecules to polymeric analytes.

FIG. 3 schematically shows an example of a sequencing approach to analyze or characterize polymeric analytes.

FIG. 4 schematically shows a computer system that is programmed or otherwise configured to implement methods provided herein.

FIG. 5 shows a reaction mechanism for conjugation to and cleavage of an N-terminal amino acid using thiourea.

FIG. 6 shows a reaction mechanism for conjugation to and cleavage of an N-terminal amino acid using a sequencing reagent comprising an azide-functionalized thiourea.

FIG. 7A shows a reaction mechanism for conjugation to and cleavage of an N-terminal amino acid using a sequencing reagent comprising 6-nitrobenzotriazole-1-carboxamidine and an azide linker. FIG. 7B shows an example synthesis scheme for the sequencing reagent of FIG. 7A.

FIG. 7C shows an example synthesis scheme for an additional sequencing reagent similar to that of FIG. 7A. FIG. 7D shows an example synthesis scheme for a similar compound as that of FIG. 7A that does not comprise an azide linker.

FIG. 8 shows a reaction mechanism for conjugation to and cleavage of an N-terminal amino acid using a sequencing reagent described herein.

FIG. 9A shows a reaction mechanism for conjugation to and cleavage of an N-terminal amino acid using another sequencing reagent described herein. FIG. 9B shows a reaction mechanism for conjugation to an N-terminal amino acid using the sequencing reagent of FIG. 9A under different reaction conditions. FIG. 9C shows LCMS data (chromatogram) of the reaction of FIG. 9B. FIG. 9D shows LCMS data (mass spectra) of the reaction of FIG. 9B.

FIG. 10A shows a synthesis scheme for the sequencing reagent illustrated in FIGS. 9A-9B. FIG. 10B shows a synthesis scheme for a precursor reagent used to generate the sequencing reagent of FIGS. 9A-9B. FIG. 10C shows NMR spectra of the resultant synthesis reaction for the precursor reagent shown in FIG. 10B. FIG. 10D shows additional NMR spectra of the resultant synthesis reaction for the precursor reagent shown in FIG. 10B. FIG. 10E shows LCMS data (chromatogram) of the resultant synthesis reaction for the precursor reagent shown in FIG. 10B FIG. 10F shows LCMS data (mass spectra) of the resultant synthesis reaction for the precursor reagent shown in FIG. 10B. FIG. 10G shows NMR spectra of the sequencing reagent of FIGS. 9A-9B, which is synthesized using the synthesis scheme of FIG. 10A. FIG. 10H shows additional NMR spectra of the sequencing reagent of FIGS. 9A-9B, which is synthesized using the synthesis scheme of FIG. 10A. FIG. 10I shows additional NMR spectra of the sequencing reagent of FIGS. 9A-9B, which is synthesized using the synthesis scheme of FIG. 10A. FIG. 10J shows an HPLC chromatogram of the sequencing reagent of FIGS. 9A-9B, which is synthesized using the synthesis scheme of FIG. 10A. FIG. 10K shows LCMS data (chromatogram) of the sequencing reagent of FIGS. 9A-9B, which is synthesized using the synthesis scheme of FIG. 10A. FIG. 10L shows LCMS data (mass spectra) of the sequencing reagent of FIGS. 9A-9B, which is synthesized using the synthesis scheme of FIG. 10A.

FIG. 11 shows additional examples of sequencing reagents described herein.

FIG. 12 shows additional example of guanidinylating agents that can be modified for use as a sequencing reagent, as described herein.

FIG. 13 shows an additional synthesis schematic of a guanidinylating agent that can be modified for use as a sequencing reagent.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to 10% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

As used herein, the term “protein” generally refers to a molecule comprising two or more amino acids joined by a peptide bond. A protein may also be referred to as a “polypeptide”, “oligopeptide”, or “peptide”. A protein can be a naturally occurring molecule, or a synthetic molecule (e.g., an artificial protein, peptide, enzyme). A protein may include one or more non-natural amino acids, modified amino acids, or non-amino acid linkers. A protein may contain D-amino acid enantiomers, L- amino acid enantiomers or both. Amino acids of a protein may be modified naturally or synthetically, such as by post-translational modifications or by chemical modification. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “peptide” may refer to any short, single peptide chain. A peptide may be no more than about 100, 95, 90, 85, 80, 75, 70, 65, 60, 55, 50, 45, 40, 35, 30, 25, 20, 15, 10, 5, or less than about 5 amino acids in length. A peptide may have a known or unknown biological function or activity. Peptides can include natural, synthetic, modified, or degraded proteins or peptides, or a combination thereof.

As used herein, the term “single analyte” may refer to an analyte that is individually manipulated or distinguished from other analytes. A single analyte may comprise a biomolecule or a synthetic molecule. A single analyte may comprise a small molecule. A single analyte can be a single molecule (e.g., a single biomolecule such as a single protein, nucleic acid molecule, affinity reagent, lipid, carbohydrate, etc.), a single complex of two or more molecules (e.g., a multimeric protein having two or more separable subunits, a single protein attached to a nucleic acid molecule or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, “polypeptide” refers to two or more amino acids linked together by a peptide bond. The term “polypeptide” includes proteins that have a C-terminal end and an N-terminal end as generally known in the art and may be synthetic in origin or naturally occurring. As used herein “at least a portion of the polypeptide” refers to 2 or more amino acids of the polypeptide. A polypeptide may comprise one or more peptides. Optionally, a portion of the polypeptide includes at least: 1, 5, 10, 20, 30 or 50 amino acids, either consecutive or with gaps, of the complete amino acid sequence of the polypeptide, or the full amino acid sequence of the polypeptide.

As used herein, the term “sample” refers to a collected substance or material that comprises or is suspected to comprise one or more analytes of interest (e.g., biomolecules, e.g., polypeptides). A sample may be modified for purposes such as storage or stability. A sample may be naturally occurring or synthetic. A sample may be processed to separate or remove unwanted fractions or impurities from the analyte(s) of interest. A sample may be enriched or purified. For example, a sample may comprise a fraction of a separation process (e.g., chromatography, fractionation, electrophoresis, etc.). Alternatively, a sample may not be subjected to processing that separates or removes any unwanted fractions or impurities from the analyte(s) of interest. A sample may be obtained from any suitable source or location, including from organisms, cells, tissues, cell preparations, cell-free compositions, the environment (e.g., air, water, dirt, soil, agriculture, soil, dust, sewage). A sample may be obtained from an organism or part of an organism, such as from a fluid, tissue, or cell. A sample may include biological and/or non-biological components. As used herein, the terms “biological sample” or “biological source” refer to a sample that is derived from a predominantly biological system or organism, such as one or more viral particles, cells (e.g. individualized cells), organelles (e.g. individualized organelles), tissues, bodily fluids, bone, cartilage, and exoskeleton. A biological sample may comprise a majority of biological material on a mass basis, excluding the weight of fluid within the sample. Biological samples may comprise one or more proteins, referred to herein as protein samples. Biological samples can be acquired from various sources, e.g., from a clinical patient sample, such as blood, serum, plasma, Cerebral Spinal Fluid (CSF), saliva, mucosal secretions, sputum, urine, lymph, perspiration, vaginal fluid, semen, fecal matter, amniotic fluid, perspiration, synovial fluid, etc. A biological sample may be processed to purify and retain one or more biomolecules (e.g., proteins, nucleic acids, carbohydrates, lipids, glycoproteins, lipoproteins, metabolites, etc.) from the biological sample. A biological sample (e.g., a protein sample) may be derived from cultured cells, which may be treated or untreated. A biological sample (e.g., a protein sample) can also result from tissue specimens, such as biopsy samples, which may optionally be processed to liberate biomolecules (e.g., proteins) contained therein. Tissue samples may also be derived from in vivo specimens, including fresh, frozen, acute, and fixed tissues.

As used herein, the terms “antibody” and “immunoglobulin” may generally refer to proteins that can recognize and bind to a specific antigen. An antibody or immunoglobulin may refer to an antibody isotype, fragments of antibodies including, but not limited to, Fab, Fv, scFv, and Fd fragments, chimeric antibodies, humanized antibodies, single-chain antibodies, and fusion proteins including an antigen-binding portion of an antibody and a non-antibody protein. The antibodies may be detectably labeled, e.g., with a fluorophore, radioisotope, enzyme (e.g., a peroxidase) which generates a detectable product, fluorescent protein, nucleic acid barcode sequence, and the like. The antibodies may be further conjugated to other moieties, such as members of specific binding pairs, e.g., biotin (member of biotin-avidin specific binding pair), and the like. Also encompassed by the terms are nanobodies, Fab′, Fv, F(ab′)₂, scFv, and other antibody fragments that retain specific binding to antigen. Antibodies may exist in a variety of other forms including, for example, Fv, Fab, and (Fab)₂, as well as bi-functional (i.e., bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., Immunology, Benjamin, N.Y., 2nd ed. (1984), and Hunkapiller and Hood, Nature, 323, 15-16 (1986), which are herein incorporated by reference). Naturally occurring immunoglobulins or antibody types include immunoglobulin A, immunoglobulin G, immunoglobulin D, immunoglobulin E, immunoglobulin M, or other immunoreactive components.

“Binding” or “coupling” as used herein generally refers to a covalent or non-covalent interaction between two molecules (referred to herein as “binding partners”, e.g., a substrate and an enzyme or an antibody and an epitope). Binding between binding partners may be specific or non-specific.

As used herein, “specifically binds” or “binds specifically” generally refers to an interaction between binding partners (e.g., a binding partner and a cognate molecule) such that the binding partners bind to one another, but do not bind to other molecules that may be present in the environment (e.g., in a biological sample, in tissue, in an in vitro assay) under a set of conditions. A specific binding interaction may entail a binding partner that binds to a cognate molecule. The specific binding interaction may entail the binding of the binding partner to its cognate molecule at a significantly or substantially higher level or with greater affinity as compared to the binding of the binding partner to a non-cognate molecule. A specific binding interaction may entail a first binding partner that has greater selectivity of binding to the cognate molecule as compared to a non-cognate molecule.

The terms “nucleic acid”, “nucleic acid molecule”, “oligonucleotide” and “polynucleotide” may be used interchangeably herein and generally refer to a polymeric form of naturally occurring or synthetic nucleotides, or analogs thereof, of any length. A nucleic acid molecule may comprise one or more deoxyribonucleotides, deoxynucleotide triphosphates, dideoxynucleotide triphosphates, ribonucleotides, hexitol nucleotides, cyclohexane nucleotides, or analogs or combinations thereof. A nucleic acid molecule may comprise, e.g., DNA, RNA, HNA, CeNA, and modified forms thereof. A nucleic acid molecule may comprise nucleotides that are linked by phosphodiester bonds. A nucleic acid molecule may have any two- or three-dimensional structure, and may perform any function, known or unknown. A nucleic acid molecule may be single stranded, double stranded, or partially double stranded. Non-limiting examples of polynucleotides include a gene, a gene fragment, exons, introns, messenger RNA (mRNA), transfer RNA, ribosomal RNA, noncoding RNA, small interfering RNA, short hairpin RNA, micro RNA, scaRNA, ribozymes, riboswitches, viral RNA, complementary DNA (cDNA), cosmid DNA, mitochondrial DNA, chromosomal or genomic DNA, viral DNA, recombinant polynucleotides, branched polynucleotides, plasmids, vectors, isolated DNA of any sequence, control regions, isolated RNA of any sequence, nucleic acid probes, nucleic acid adapters, and primers. The nucleic acid molecule may be linear, circular, or any other geometry. Examples of polynucleotide analogs include but are not limited to xeno nucleic acid (XNA), bridged nucleic acid (BNA), glycol nucleic acid (GNA), hexitol nucleic acid (HNA), cyclohexane nucleic acid (CeNA), 2′-F-Arabinonucleic acids (2′-F-ANA), peptide nucleic acids (PNAs), yPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding.

As used herein, the term “amino acid” generally refers to an organic compound that combines to form a protein or peptide. An amino acid generally comprises an amine group, a carboxylic acid group, and a side-chain specific to each amino acid, which serve as a monomeric subunit of a peptide. An amino acid may include the 20 standard, naturally occurring or canonical amino acids as well as non-standard amino acids. The standard, naturally-occurring or canonical amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, (3-amino acids, Homo-amino acids, Proline and Pyruvic acid derivatives, 3-substituted alanine derivatives, glycine derivatives, ring-substituted phenylalanine and tyrosine derivatives, linear core amino acids, and N-methyl amino acids.

As used herein, the term “amino acid type” generally refers to one of the standard, naturally-occurring or canonical amino acids, e.g., one member of the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), Tyrosine (Y or Tyr), derivatives thereof, and modified forms of any of the aforementioned amino acids. The term “amino acid type” may be used herein to distinguish a plurality of amino acids that comprise different side chain groups, rather than a plurality of amino acids that are identical (e.g., different positional amino acids of a single peptide that have the same side chain).

As used herein, the term “post-translational modification” refers to modifications that occur on a peptide subsequent to translation. A post-translational modification may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, transglutamination, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, sumoylation, disulfide bond formation, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini. The term post-translational modification can also include peptide modifications that include one or more detectable labels. A post-translational modification may be naturally occurring or synthetic.

As used herein, the term “binding agent” refers to a molecule, e.g., a nucleic acid molecule, a peptide, a polypeptide, a protein, carbohydrate, a synthetic molecule, or a small molecule that binds to, associates with, unites with, recognizes, or combines with another molecule. The binding agent may bind to a macromolecule or a component or feature of a macromolecule. A binding agent may form a covalent association or non-covalent association with a molecule, a macromolecule, or a component or feature of a macromolecule. A binding agent may also be a chimeric binding agent, composed of two or more types of molecules, such as a nucleic acid molecule-peptide chimeric binding agent, a carbohydrate-peptide chimeric binding agent, or a lipid-peptide chimeric binding agent. A binding agent may be a naturally occurring, synthetically produced, or recombinantly expressed molecule. A binding agent may bind to a single monomer or subunit of a polymeric analyte, such as a macromolecule (e.g., a single amino acid of a peptide) or bind to a plurality of linked subunits of a macromolecule (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferably bind to a chemically modified or labeled amino acid over a non-modified or unlabeled amino acid. For example, a binding agent may preferably bind to an amino acid that has been modified with an acetyl moiety, guanyl moiety, dansyl moiety, PTC moiety, DNP moiety, SNP moiety, etc., over an amino acid that does not possess such a moiety. A binding agent may bind to a post-translational modification, either naturally occurring or synthetic, of a peptide molecule. A binding agent may exhibit selective binding to a component or feature of a macromolecule (e.g., a binding agent may selectively bind to one of the 20 possible natural amino acid residues and with bind with very low affinity or not at all to the other 19 natural amino acid residues). A binding agent may exhibit less selective binding, where the binding agent is capable of binding a plurality of components or features of a macromolecule (e.g., a binding agent may bind with similar affinity to two or more different amino acid residues). A binding agent may comprise a tag, which may be coupled to the binding agent via a linker.

As used herein, the term “linker” generally refers to a molecule or moiety that is involved in joining two or more molecules. A linker may facilitate a covalent or noncovalent interaction of two or more molecules. A linker may be a crosslinker. The linker can be unifunctional, bifunctional, trifunctional, quadrifunctional, or polyfunctional. A linker can be or comprise a nucleotide, a nucleotide analog, an amino acid, a peptide, a polypeptide, or a non-nucleotide chemical moiety, such as an organic or inorganic compound. A linker may comprise a polymer, such as a polyethylene glycol (PEG), poly-L-lysine (PLL), poly (DL-lactic acid) (PLA), poly (DL-lactide-co-glycoside) (PLGA), polyomithine, polyarginine, etc. A linker may comprise one or more reactive ends, e.g., an amine-reactive group, a carboxyl-reactive group, a sulfhydryl-reactive group, a hydroxyl-reactive group, etc. In some examples, a linker may be used to join different molecule types, e.g., different biomolecule types such as a peptide with a nucleic acid molecule, a lipid with a peptide, a carbohydrate with a peptide, etc.; non-biomolecule types; or a biomolecule to a non-biomolecule. For example, a linker may be used to join a binding agent with a tag, a tag with a macromolecule (e.g., peptide, nucleic acid molecule), a macromolecule with a solid support, a tag with a solid support, etc. A linker may join two molecules via enzymatic reaction or chemistry reaction (e.g., click chemistry). A linker may join more than two molecules, e.g., via enzymatic or chemical reactions.

The term “conjugated” as used herein generally refers to a covalent or ionic interaction between two entities, e.g., molecules, compounds, or combinations thereof.

As used herein, the term “tag” generally refers to a molecule or moiety that is conjugated to a molecule. A tag may comprise a detectable label, e.g., a fluorophore or fluorescent protein, a radioactive isotope, an enzyme (e.g., a chromogenic or fluorescent protein, proteins that can catalyze chromogenic substrates), a mass tag, a hapten (e.g., biotin, digoxigenin, urushiol, fluorescein), a vibrational or FTIR tag (e.g., alkyne group). A tag may comprise a biomolecule, such as a nucleic acid molecule, a protein, a lipid, a carbohydrate, or a combination thereof. A tag may comprise one or more nucleic acid molecules, which may optionally encode information regarding the tag or the molecule onto which a tag is conjugated (e.g., a binding agent, such as an antibody). For example, a tag may comprise a nucleic acid barcode molecule. A tag may comprise an organic compound or an inorganic compound.

As used herein, the term “barcode” generally refers to an identifying feature that may be used to distinguish similar items. A barcode may comprise a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 bases), which may provide a unique identifier tag or origin information for a molecule (e.g., protein, polypeptide, peptide), a binding agent, a set of binding agents from a binding cycle, a sample molecule, a set of samples, molecules within a compartment (e.g., droplet, bead, partition or separated location), macromolecules within a set of compartments, a fraction of macromolecules, a set of macromolecule fractions, a spatial region or set of spatial regions, a library of macromolecules, or a library of binding agents. A barcode can be an artificial sequence or a naturally occurring sequence including peptides, proteins, protein complexes, carbohydrates, and synthetic polymeric materials. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. A population of barcodes may comprise error correcting barcodes. Barcodes can be used to computationally deconvolute sequence reads derived from an individual molecule, sample, library, etc. Barcodes may comprise multiplexed information, e.g., arising from different samples, compartments, individual molecules, etc. A barcode can also be used for deconvolution of a collection of molecules that have been distributed into small compartments for enhanced mapping. For example, rather than mapping a peptide back to the proteome, the peptide can be mapped back to its originating protein molecule or protein complex. A barcode may comprise any useful structure moiety or motif, e.g., hairpins, loop sequences, or spacers. Barcodes can comprise artificial or modified nucleic acids, e.g., locked nucleic acids (LNA), protein nucleic acids (PNA), hexitol nucleic acids (HNA), cyclohexane nucleic acids (CeNA), or a combination thereof. Barcodes may comprise or be generated using a protein, e.g., Tal effector, Cas protein (e.g., Cas9), Argonaut, or coiled coils.

As used herein, a “sample barcode”, also referred to as “sample tag” generally refers to a barcode molecule comprising identifying information of a sample from which a barcoded molecule derives.

As used herein, a “spatial barcode” generally refers to a barcode molecule comprising identifying information of a region of a 2-D or 3-D sample (e.g., a tissue section) from which a molecule originates or is derived. Spatial barcodes may be used for molecular pathology on tissue sections. A spatial barcode may allow for multiplex sequencing of a plurality of samples or libraries from tissue section(s).

As used herein, a “temporal barcode” generally refers to a barcode molecule comprising time-based information relating to the barcoded molecule. The types of time-based data encoded in a temporal barcode can include information such as a lifetime of a barcoded molecule, a time of collection of a sample, a time or duration since the beginning of an experiment or induction with a stimulus, information on the age of a cell or tissue, a sequence of interactions between molecules, among others. It is possible for different types of barcodes (e.g., spatial, temporal, cell-specific) to be combined in one multiplexed barcode.

As used herein, the term “nucleic acid sequence” or “oligonucleotide sequence” generally refers to a contiguous string of nucleotide bases and may refer to the particular placement of nucleotide bases in relation to each other as they appear in an oligonucleotide. Similarly, the term “polypeptide sequence” or “amino acid sequence” refers to a contiguous string of amino acids and may refer to the particular placement of amino acids in relation to each other as they appear in a polypeptide.

A “nucleic acid molecule” according to the present invention may include any polymer or oligomer of nucleotides such as pyrimidine and purine bases, such as cytosine, thymine, and uracil, and adenine and guanine, respectively and combinations thereof. The nucleotide sequence may comprise any deoxyribonucleotide, ribonucleotide, hexitol-nucleotide, cyclohexane-nucleotide, peptide nucleic acid component, and any chemical variants thereof, such as methylated, 7-deaza purine analogs, 8-halopurine analogs, hydroxymethylated or glycosylated forms of these bases, and the like. The polymers or oligomers may be heterogeneous or homogenous in composition and may be isolated from naturally occurring sources or may be artificially or synthetically produced. A nucleic acid molecule may comprise DNA, RNA, HNA, CeNA or a mixture thereof, and may exist permanently or transitionally in single-stranded or double-stranded form, including homoduplex, heteroduplex, and hybrid states.

The terms “complementary” or “complementarity” refer to polynucleotides (i.e., a sequence of nucleotides) related by Watson-Crick base-pairing rules. For example, the sequence “5′-AGT-3′,” is complementary to the sequence “5′-ACT-3”. Complementarity may be “partial,” in which only some of the nucleic acids' bases are matched according to the base pairing rules, or there may be “complete” or “total” complementarity between the nucleic acids. The degree of complementarity between nucleic acid strands can have significant effects on the efficiency and strength of hybridization between nucleic acid strands under defined conditions.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the melting temperature of the formed hybrid. Hybridization methods involve the annealing of one nucleic acid to another, complementary nucleic acid, e.g., based on Watson-Crick base pairing.

As used herein, the term “proteomics” generally refers to quantitative and/or qualitative analysis of the proteome within a sample, such as biological sample, e.g., from cells, tissues, or bodily fluids. Proteomics may include the analysis of spatial distributions of proteins within a sample (e.g., cell and/or tissues). Proteomics may include studies of the dynamic state of the proteome, e.g., how one or more proteins change in time. A proteome may comprise multiple “-omes”, e.g., a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof.

The terminal amino acid at one end of the peptide chain that has a free amino group may be referred to herein as the “N-terminal amino acid” (NTAA). The terminal amino acid at the other end of the chain that has a free carboxyl group may be referred to herein as the “C-terminal amino acid” (CTAA). The amino acids making up a peptide may be numbered in order, with the peptide being “n” amino acids in length. As used herein, in some instances, NTAA may be considered the nth amino acid (also referred to herein as the “n NTAA”). In such cases, the next amino acid is the n-1 amino acid, then the n-2 amino acid, and so on down the length of the peptide from the N-terminal end to C-terminal end. Alternatively, CTAA may be considered the nth amino acid (also referred to herein as the “n CTAA”). In such cases, the next amino acid is the n-1, then the n-2 amino acid, and so on down the length of the peptide from the C-terminal end to N-terminal end. An NTAA, CTAA, or both may be modified or labeled with a chemical moiety.

As used herein, the terms “determining,” “measuring,” “assessing,” and “assaying” are used interchangeably and include both quantitative and qualitative determinations.

As used herein, the term “unique molecular identifier” or “UMI” generally refers to a molecule barcode comprising indexing information. A UMI may comprise a nucleic acid molecule of about 3 to about 150 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 bases) in length. A UMI may provide a unique identifier tag for each molecule (e.g., peptide, binding agent, a nucleic acid molecule) that comprises or is coupled to a UMI. A UMI may comprise a random sequence (e.g., a random N-mer).

As used herein, a “derivative” of a nucleic acid molecule generally refers to a nucleic acid molecule that is derived from an originating nucleic acid molecule. The derivative may have the same or substantially the same nucleotide sequence as the originating nucleic acid molecule, or the derivative may comprise a complement or partial complement as the originating nucleic acid molecule. A derivative may be the same type of nucleic acid (e.g., DNA or RNA) as the originating nucleic acid molecule, or the derivative may be a different type of nucleic acid (e.g., cDNA generated from an RNA molecule). A nucleic acid molecule derivative may display sequence identity as the originating nucleic acid molecule. The derivative nucleic acid molecule may also be subjected to additional processing from the originating nucleic acid molecule, e.g., chemical or enzymatic modification, splicing, ligation, polymerization, fragmentation, tagmentation (e.g., using a transposase), digestion, etc.

A derivative polypeptide or peptide may be derived from an originating polypeptide (or peptide). A derivative may comprise the same amino acid sequence as the originating polypeptide, or the sequence may be different. The derivative polypeptide may result from or be subjected to additional processing from the originating polypeptide, e.g., chemical or enzymatic modification. The derivative polypeptide may comprise one or more tags, nucleic acid molecules, barcode molecules, labels (e.g., detectable labels), fluorophores, probes, linkers, post-translational modifications, chemical protecting groups, or other chemical moieties.

As used herein, the term “compartment” or “partition” generally refers to a physical area or volume that separates or isolates a subset of molecules from a sample of molecules. For example, a compartment may separate an individual cell from other cells, or a subset of a sample's proteome from the rest of the sample's proteome. A compartment may be an aqueous compartment (e.g., microfluidic droplet), a solid compartment (e.g., picotiter well or microtiter well on a plate, tube, vial, gel bead), or a separated region on a surface. A compartment may comprise one or more beads to which macromolecules may be immobilized.

In some embodiments, the partitions as described herein are droplets. The terms “drop,” “droplet,” and “microdroplet” are used interchangeably herein, to refer to small, generally spherically structures, containing at least a first fluid phase, e.g., an aqueous phase (e.g., water), bounded by a second fluid phase (e.g., oil) which is immiscible with the first fluid phase. In some embodiments, droplets according to the present disclosure may contain a first fluid phase, e.g., oil, bounded by a second immiscible fluid phase, e.g., an aqueous phase fluid (e.g., water). In some embodiments, the second fluid phase will be an immiscible phase carrier fluid. Thus, droplets according to the present disclosure may be provided as aqueous-in-oil emulsions or oil-in-aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, reagents, and a variety of other components. The term droplet may be used to refer to a droplet produced in, on, or by a microfluidic device and/or flowed from or applied by a microfluidic device.

As used herein, the term “carrier fluid” refers to a fluid configured or selected to contain one or more discrete entities, e.g., droplets, as described herein. A carrier fluid may include one or more substances and may have one or more properties, e.g., viscosity, which allow it to be flowed through a microfluidic device or a portion thereof, such as a delivery orifice. In some embodiments, carrier fluids include, for example: oil or water, and may be in a liquid or gas phase. Suitable carrier fluids are described in greater detail herein.

As used herein, the term “solid support”, “solid surface”, or “solid substrate” or “substrate” refers to any solid material, including porous and non-porous materials, to which a molecule can be associated directly or indirectly. The molecule may be associated with the substrate by covalent or non-covalent interactions, or a combination thereof A substrate may be two-dimensional (e.g., planar surface) or three-dimensional (e.g., gel matrix or bead). A solid support may comprise, in non-limiting examples, a bead, a microbead, an array, a glass surface, a silicon surface, a plastic surface, a filter, a membrane, nylon or other polymer, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polylactic acid, polyorthoesters, functionalized silane, polypropylfumerate, collagen, glycosaminoglycans, polyamino acids, dextran, or any combination thereof Solid supports further include thin film, membrane, bottles, dishes, fibers, woven fibers, shaped polymers such as tubes, particles, beads, microspheres, microparticles, or any combination thereof For example, when solid surface is a bead, the bead can include, but is not limited to, a ceramic bead, polystyrene bead, a polymer bead, a methylstyrene bead, an agarose bead, an acrylamide bead, a solid core bead, a porous bead, a magnetic or paramagnetic bead, a glass bead, or a controlled pore bead. A bead may be spherical or an irregularly shaped. A bead's size may range from nanometers, e.g., 100 nm, to millimeters, e.g., 1 mm. In certain embodiments, beads range in size from about 0.2 micron to about 200 microns, or from about 0.5 micron to about 5 microns. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μm in diameter. In certain embodiments, “a bead” solid support may refer to an individual bead or a plurality of beads.

As used herein, “sequencing” generally refers to determining the order of: (A) nucleotides (base sequences) in a nucleic acid sample, e.g., DNA or RNA; or determining the order of (B) amino acids in all or part of a polymer, such as a protein, peptide, or other multimeric molecule. Many techniques are available, such as Sanger sequencing or High Throughput Sequencing technologies (HTS). Sanger sequencing may involve sequencing via detection through (capillary) electrophoresis, in which up to 384 capillaries may be sequence analyzed in one run. High throughput sequencing involves the parallel sequencing of thousands or millions or more sequences at once. HTS can be defined as Next Generation sequencing (NGS), i.e. techniques based on solid phase pyrosequencing or as Next-Next Generation sequencing based on single nucleotide real time sequencing (SMRT). HTS technologies are available such as offered by Roche, lllumina and Applied Biosystems (Life Technologies). Further high throughput sequencing technologies are described by and/or available from Helicos, Pacific Biosciences, Complete Genomics, Ion Torrent Systems, Oxford Nanopore Technologies, Nabsys, ZS Genetics, GnuBio.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, nanopore sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, ThermoFisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science 311:1544-1546, 2006).

As used herein, “analyzing” the macromolecule means to quantify, characterize, distinguish, or a combination thereof, all or a portion of the components of a molecule (e.g., a macromolecule, a biological molecule such as a protein, amino acid, nucleic acid molecule, etc.). For example, analyzing a peptide, polypeptide, or protein may comprise determining all or a portion of the amino acid sequence (contiguous or non-continuous) of the peptide. Analyzing a macromolecule may include partial identification of a component of the macromolecule. For example, partial identification of amino acids in a protein sequence can identify an amino acid in the protein as belonging to a subset of possible amino acids. Analysis may be performed sequentially, e.g., beginning with analysis of the n NTAA, and then proceeding to the next amino acid of the peptide (i.e., n-1, n-2, n-3, and so forth). In such instances, sequencing may be performed by cleavage of the n NTAA, thereby converting the n-1 amino acid of the peptide to an N-terminal amino acid (referred to herein as the “n-1 NTAA”). Similarly, analysis of a peptide may begin from C-terminus towards the N-terminus with each round of cleavage from the C-terminus creating a new CTAA. Cleavage of the n CTAA converts the n-1 amino acid of the peptide to a C-terminal amino acid, referred to herein as an “n-1 CTAA”. Analyzing the peptide may also include determining a presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

As used herein, the term “analyte” generally refers to a substance that is of interest to be further identified, characterized, or measured. An analyte can be, in non-limiting examples, an ion, chemical, compound, small molecule, element, particle, metal, biomolecule, macromolecule, metabolite, lipid, carbohydrate, peptide or protein, nucleic acid molecule, organelle, or cell. An analyte may be naturally occurring or synthetic. The analyte may be a solid, semi-solid, liquid, semi-liquid, gas, or plasma. The analyte may be characterized qualitatively or quantitatively. A portion of an analyte may be analyzed. For example, an analyte may be a peptide and the constituent amino acids may be analyzed. The analyte may comprise a polymer, also referred to herein as “polymeric analyte”, which generally refers to an analyte of interest that comprises one or more monomers. A polymeric analyte can be, in non-limiting examples, a group of ions, chemicals, compounds, small molecules, elements, particles, metals, or a biomolecule, macromolecule, metabolite, lipid, carbohydrate, peptide or protein, nucleic acid molecule, organelle, or cell.

As used herein, the term “array” generally refers to a population of molecules that is attached to one or more solid supports such that the molecules at one address can be distinguished from molecules at other addresses. An array can include different molecules that are each located at different addresses on a solid support. Alternatively, an array can include separate solid supports each functioning as an address that bears a different molecule, wherein the different molecules can be identified according to the locations of the solid supports on a surface to which the solid supports are attached, or according to the locations of the solid supports in a liquid such as a fluid stream. The molecules of the array can be, for example, nucleic acids such as SNAPs, polypeptides, proteins, peptides, oligopeptides, enzymes, ligands, or receptors such as antibodies, functional fragments of antibodies or aptamers. The addresses of an array can optionally be optically observable, and, in some configurations, adjacent addresses can be optically distinguishable when detected using a method or apparatus set forth herein.

As used herein, the term “functionalized” refers to any material or substance that has been modified to include a functional group. A functionalized material or substance may be naturally or synthetically functionalized. For example, a polypeptide can be naturally functionalized with a phosphate group, oligosaccharide (e.g., glycosyl, glycosylphosphatidylinositol or phosphoglycosyl), nitrosyl, methyl, acetyl, lipid (e.g., glycosyl phosphatidylinositol, myristoyl or prenyl), ubiquitin or other naturally occurring post-translational modification. A functionalized material or substance may be functionalized for any given purpose, including altering chemical properties (e.g., altering hydrophobicity or changing surface charge density) or altering reactivity (e.g., capable of reacting with a moiety or reagent to form a covalent bond to the moiety or reagent).

As used herein, the term “click reaction,” “click chemistry,” or “bioorthogonal reaction” refers to single-step, thermodynamically favorable conjugation reaction utilizing biocompatible reagents. A click reaction may utilize no toxic or biologically incompatible reagents (e.g., acids, bases, heavy metals) or generate no toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −50 kJ/mol, −100 kJ/mol, −200 kJ/mol, −300 kJ/mol, −400 kJ/mol, or less than −500 kJ/mol. Exemplary bioorthogonal and click reactions are described in detail in WO 2019/195633A1, which is herein incorporated by reference in its entirety. Exemplary click reactions may include metal-catalyzed azide-alkyne cycloaddition, strain-promoted azide-alkyne cycloaddition, strain-promoted azide-nitrone cycloaddition, strained alkene reactions, thiolene reaction, Diels-Alder reaction, inverse electron demand Diels-Alder reaction, [3+2]cycloaddition, [4+1]cycloaddition, nucleophilic substitution, dihydroxylation, thiolyne reaction, photoclick, nitrone dipole cycloaddition, norbornene cycloaddition, oxanobomadiene cycloaddition, tetrazine ligation, and tetrazole photoclick reactions. Exemplary functional groups or reactive handles utilized to perform click reactions may include alkenes (e.g., linear alkenes or cyclic alkenes such as trans-cyclooctene (TCO)), alkynes (e.g., linear alkynes or cycloalkynes (e.g., cyclooctynes or derivatives thereof, e.g., aza-dimethoxycyclooctyne (DIMAC), symmetrical pyrrolocyclooctyne (SYPCO), pyrrolocyclooctyne (PYRROC), difluorocyclooctyne (DIFO), α,α-bis(trifluoromethyl)pyrrolocyclooctyne (TRIPCO), bicyclo[6.1.0] nonyne (BCN), dibenzocyclooctyne (DIBO), difluorinated cyclooctyne (DIFO), difluorobenzocyclooctyne (DIFBO), dibenzoazacyclo-octyne (DBCO), difluoro-aza-dibenzocyclooctyne (F2-DIBAC), biaryl-azacyclooctynone (BARAC), difluorodimethoxydibenzocyclooctynol (FMDIBO), difluorodimethoxydibenzocyclooctynone (keto-FMDIBO), and 3,3,6,6-tetramethylthiacycloheptyne (TMTH)), TMTH-sulfoximine (TMTHSI), azides, epoxides, amines, thiols, nitrones, isonitriles, isocyanides, aziridines, activated esters, and tetrazines, triazoles, and combinations, variations, or derivatives thereof The click chemistry moieties may be subjected to conditions sufficient to react the first click chemistry moiety to the second click chemistry moiety, e.g., provision of metal catalysts, appropriate solvents, pH, temperature, ionic concentration, or light/energy, for any useful duration of time.

As used herein, the terms “group” and “moiety” are intended to be synonymous when used in reference to the structure of a molecule. The terms refer to a component or part of the molecule. The terms do not necessarily denote the relative size of the component or part compared to the molecule, unless indicated otherwise. The terms do not necessarily denote the relative size of the component or part compared to any other component or part of the molecule, unless indicated otherwise. A group or moiety can contain one or more atoms.

As used herein, “primers” generally refer to nucleic acid molecules which can prime the synthesis of a nucleic acid molecule (e.g., DNA or RNA). A primer may be single stranded. A primer may comprise one or more recognition sites for a protein (e.g., a polymerizing enzyme, a restriction enzyme, a cleaving enzyme, a nuclease, etc.) to bind to the primer or a primer hybridized to a template strand. A primer may comprise DNA, RNA, or other nucleic acid analogs or noncanonical bases (e.g., spacer moieties, uracils, abasic sites). A primer may optionally comprise any number of functional sequences such as sequencing primer sequences (e.g., P5 or P7 sequences), sequencing primer-binding sequences, read sequences (e.g., R1 or R2 sequences), restriction sites, nuclease-recognition sites, abasic sites, cleavage sites, transposition sites, a barcode sequence, a unique molecular identifier (UMI), etc.

“Amplification” or “amplifying” generally refers to a polynucleotide amplification reaction, namely, a population of polynucleotides that are replicated from one or more starting sequences. Amplifying may refer to a variety of amplification reactions, including but not limited to polymerase chain reaction (PCR), linear polymerase reactions, nucleic acid sequence- based amplification, rolling circle amplification and similar reactions. An amplification reaction may generate an amplicon.

An “adapter” as referred to herein, generally refers to a short nucleic acid molecule (e.g., about 10 to about 100 base pairs in length). An adapter may comprise a short double-stranded DNA molecule. An adapter may be attached, e.g., via polymerization or ligation, to an end of a DNA fragments or amplicons. Adapters may comprise synthetic oligonucleotides, e.g., oligonucleotides that have nucleotide sequences which are at least partially complementary to each other. An adapter may have blunt ends, may have staggered ends (also referred to herein as a 3′ or 5′ “overhang sequence” or “sticky end”, or a blunt end and a staggered end. Adapters may be attached (e.g., via ligation) to fragments to provide an adapter-ligated fragment; the adapter-ligated fragment may serve as a starting point for subsequent manipulation e.g., for amplification or sequencing. An adapter may be functionalized, e.g., conjugated with a tag, probe, detectable label, affinity capture reagent (e.g., biotin or streptavidin).

The term “capture moiety” as used herein generally refers to a molecule that is configured to be coupled to another moiety or molecule. A capture moiety can be a biomolecule, e.g., a lipid, carbohydrate, sugar, amino acid, peptide or protein, nucleotide, nucleic acid molecule, metabolite, or a combination thereof (e.g., glycoproteins, lipoproteins, glycosaminoglycans, etc.). A capture moiety can be a small molecule, organic compound, inorganic compound, metal, polymer, ion, or other molecule or molecular compound. A capture moiety may comprise a macromolecule. A capture moiety may comprise an enzyme, antibody, antibody fragment, nanobody, aptamer, biotin, streptavidin, avidin, neutravidin, or analogs or derivatives thereof. A capture moiety may comprise more than one molecule, e.g., a dimer, trimer, tetramer, pentamer, hexamer, heptamer, octamer, etc. A capture moiety can be a solid substrate or part of a solid substrate, or the capture moiety can be separate from a substrate, e.g., in a fluidic medium (e.g., air, in a liquid solution). A capture moiety may be coupled to an analyte (e.g., a polymeric analyte). A capture moiety may have specificity to a binding partner or a plurality of binding partners. A capture moiety may be able to bind to one molecule or moiety (univalent), or a plurality of molecules or moieties (multivalent).

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R). References to these amino acids are also in the form of “[amino acid][residues/residues]” (e.g., lysine residue, lysine residues, leucine residue, leucine residues, etc.).
“Amino” refers to the —NH₂ radical.
“Cyano” refers to the —CN radical.
“Nitro” refers to the —NO₂ radical.
“Oxo” refers to the =0 radical.
“Hydroxyl” refers to the —OH radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C₁₅ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent but may also be divalent (which may also be described herein as “alkylene” or “alkylenyl” groups). In certain embodiments, an alkyl comprises one to thirteen carbon atoms (e.g., C₁-C₁₃ alkyl). In certain embodiments, an alkyl comprises one to eight carbon atoms (e.g., C₁-C₅ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OW, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is optionally substituted as described for “alkyl” groups.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is optionally substituted as described for alkyl groups herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hickel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbomyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OW, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R is an alkylene chain as defined above. The alkylene chain and the carbocyclyl radical is optionally substituted as defined above.

“Carbocyclylalkenyl” refers to a radical of the formula —R^{c}-carbocyclyl where R^{c} is an alkenylene chain as defined above. The alkenylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, heteroalkyl does not include alkoxy as defined herein. Unless stated otherwise specifically in the specification, a heteroalkyl group is optionally substituted as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated. In some instances, the heterocyclyl radical is saturated and substituted. In some instances, the heterocyclyl radical is unsaturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3-or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Huckel theory. Heteroaryl includes fused or bridged ring systems. The heteroatom(s) in the heteroaryl radical is optionally oxidized. One or more nitrogen atoms, if present, are optionally quatemized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h] cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10α-octahydrobenzo[h]quinazolinyl, 1-phenyl-TH-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)OR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

### Single-Molecule Sequencing Reagents

The present disclosure provides novel approaches and compounds to sequence polymeric analytes, e.g., peptides, that comprise individual monomers, e.g., amino acids. In the approaches described herein, the individual monomers (e.g., terminal amino acids from a peptide) are isolated using a sequencing reagent of Formula (I) by tethering a monomer (e.g., a terminal amino acid) to a capture moiety using the sequencing reagent, cleaving the monomer from the polymeric analyte (e.g., peptide), and detecting the sequencing reagent-monomer complex or derivative thereof. In some embodiments, the detection is performed using binding agents that are specific to the sequencing reagent-monomer complexes (e.g., sequencing reagent-amino acid complexes) or derivative thereof Alternatively or in addition to, the sequencing reagent-monomer complexes may be directly detected, e.g., using a nanopore. For polymeric analytes comprising peptides, the isolation or separation of amino acids provided herein may avoid a local environment problem in which adjacent amino acids can impact the detection of the amino acids. For instance, a binding property (e.g., selectivity or affinity) of a binding agent can be negatively impacted by adjacent or neighboring amino acids. One or more approaches described herein may be performed in solution, using a substrate, or a combination thereof.

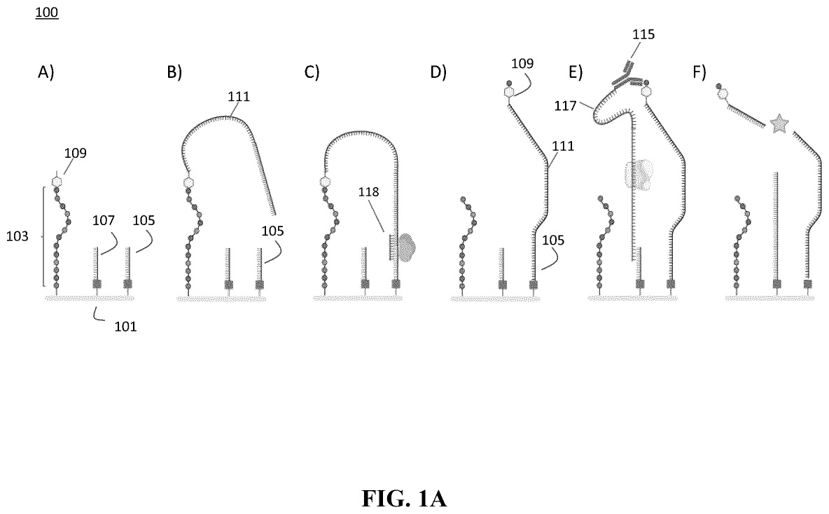

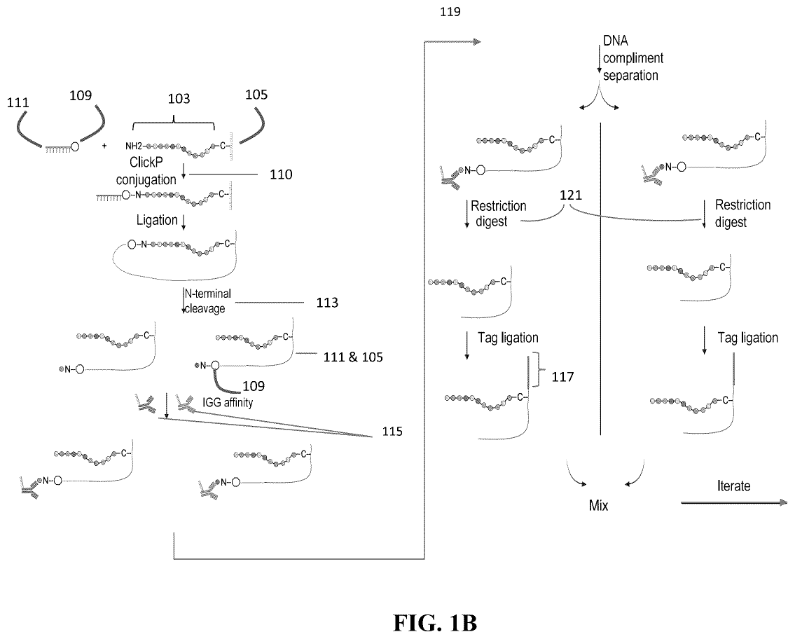

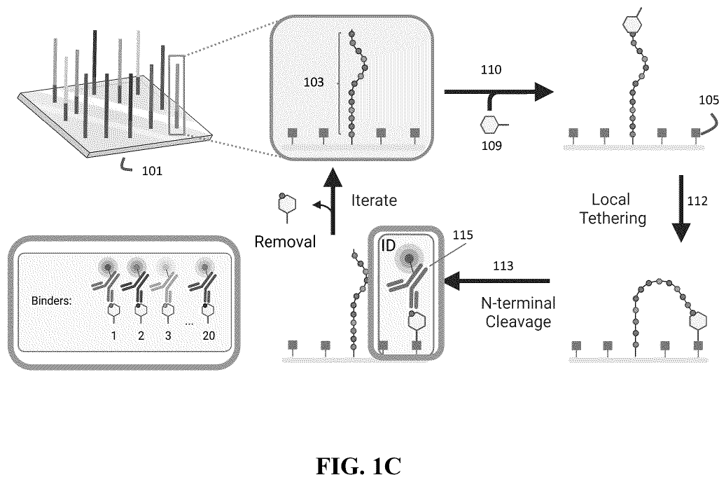

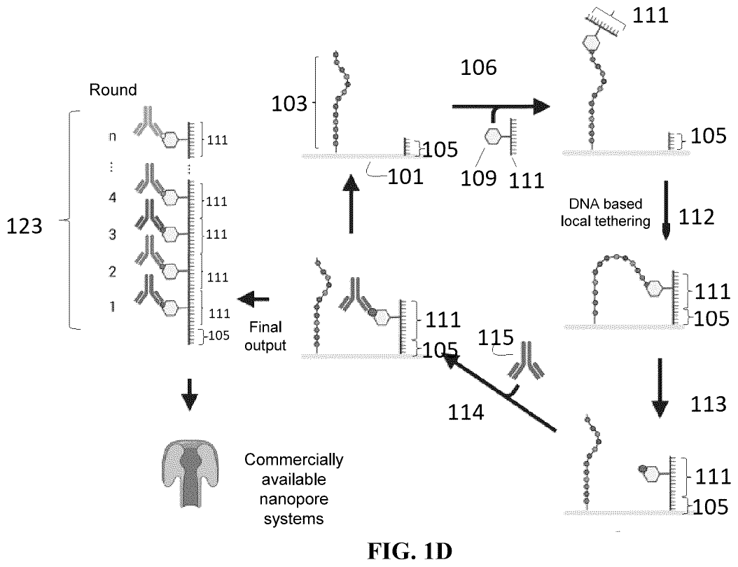

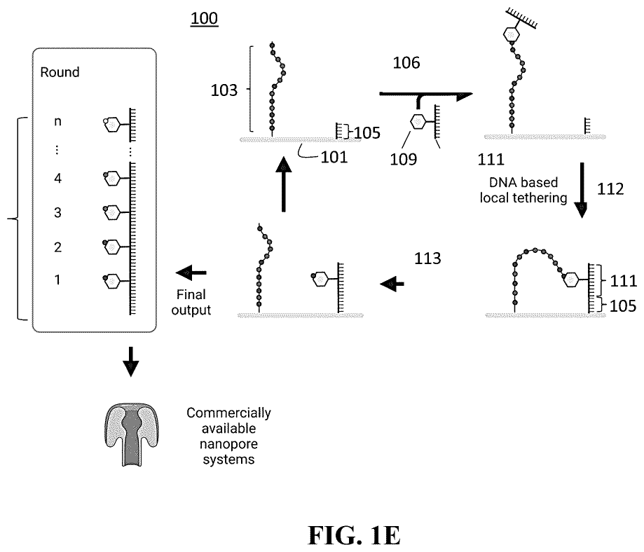

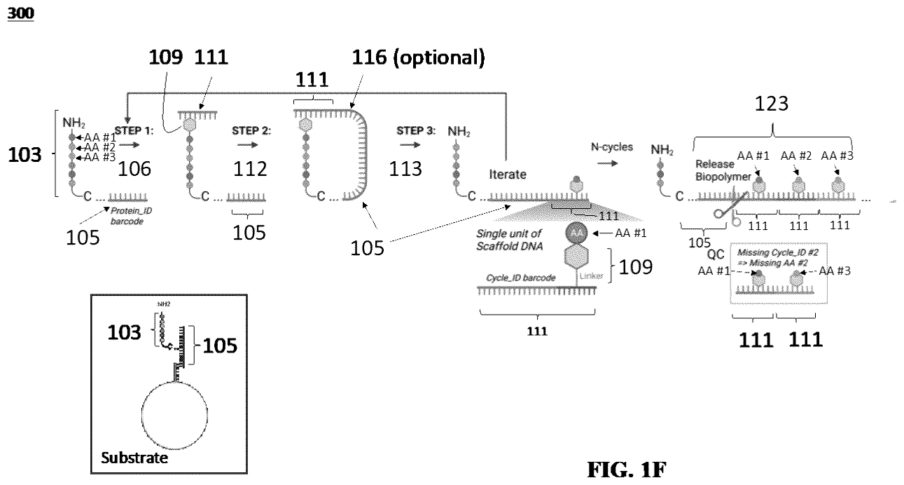

Classical peptide sequencing may be performed using Edman degradation, in which the N-terminal amino acid of a peptide is sequentially removed. This is completed using a phenyl isothiocyanate reactive group (PITC), which upon reaction with the N-terminal amino acid, results in a phenylthiocarbamoyl intermediate which is cleaved, creating a cyclic anilinothiazolinone (ATZ) compound. Sequential removal of amino acid residues allows for peptide sequencing without damage of the peptide or protein itself. However, there are a number of issues associated with Edman degradation and the use of PITC as a reactive group. These issues comprise the requirement of harsh reaction conditions (e.g., high heat and acid). These harsh reaction conditions are not amenable to the analysis or use of nucleic acid molecules (e.g., DNA used for barcoding or coupling of monomers to a substrate, described elsewhere herein and exemplified in FIGS. 1A-1F). Further, PITC can react with certain amino acids comprising a primary amine side chain, such as lysine, thereby generating a PITC-conjugated amino acid that may require additional processing for detection or require that the detection method be able to detect the PITC-conjugated amino acid.

Provided herein, in some embodiments, is a sequencing reagent comprising a reactive group. In some embodiments, the sequencing reagent comprises a capture-binding moiety. In some embodiments, the sequencing reagent comprises a linker coupled to the reactive group and the capture-binding moiety. In some instances, the sequencing reagents provided herein comprise a guanidinylating agent. The sequencing reagents provided herein can allow for sequential removal of terminal amino acid residues under milder reaction conditions than the high heat and acidic conditions required for Edman degradation. Such milder reaction conditions may be amendable to use of polymerizable molecules, such as nucleic acid molecules, that can be used for further analysis of the removed terminal amino acid residues. In some examples, the sequencing reagents provided herein provide high efficiency, such as faster kinetics than that of traditional Edman degradation.

In some embodiments, provided herein is a sequencing agent of Formula I:

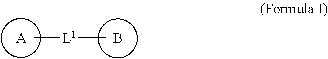

or a stereoisomer, tautomer, or salt thereof. In some embodiments, A comprises a reactive group configured to couple to a monomer of a polymeric analyte. In some embodiments, A comprises an amino acid-reactive guanidinylating agent that is configured to form a covalent bond with an N-terminal amino acid (monomer) of a peptide (a polymeric analyte). In some embodiments, A comprises a reactive group as described elsewhere herein, such as a reactive group of Formula I-B, II-A, or III-A. In some embodiments, B comprises a capture-binding moiety, which, in some instances, may act as a substrate-tethering moiety. The capture-binding moiety may be coupled to or configured to couple to a capture moiety. In some embodiments, B comprises a click chemistry moiety (e.g., azide, alkyne including cycloalkynes). In some embodiments, B comprises an azide. In some embodiments, B comprises an alkyne. In some embodiments, B comprises a nucleic acid molecule. In some embodiments, L¹ comprises a linker coupled to A and B.

In some embodiments, provided herein is a sequencing reagent comprising a compound of Formula IV:

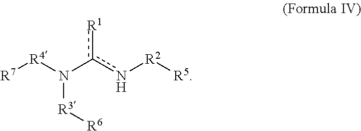

In some embodiments, R¹ comprises one or more leaving groups. In some embodiments, a leaving group comprises an electrophilic group. In some embodiments, the electrophilic group comprises S, SCH₃, SO₃H, or NHTf. In some embodiments, the electrophilic group comprises S. In some embodiments, the electrophilic group comprises SCH₃. In some embodiments, the electrophilic group comprises SO₃H. In some embodiments, the electrophilic group comprises NHTf. In some embodiments, the electrophilic group is substituted or unsubstituted C₃₋₁₀ aryl or substituted or unsubstituted C₃₋₁₀ heteroaryl. In some embodiments, the electrophilic group is substituted or unsubstituted pyrazole or triazole. In some embodiments, the electrophilic group is substituted or unsubstituted pyrazole. In some embodiments, the electrophilic group is substituted or unsubstituted triazole.

In some embodiments, R¹ is:

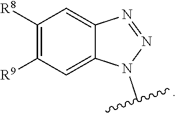

In some embodiments, R⁸ and R⁹ are independently selected from hydrogen or an electron withdrawing group. In some embodiments, R⁸ is hydrogen. In some embodiments, R⁹ is hydrogen. In some embodiments, R⁸ is an electron withdrawing group. In some embodiments, R⁹ is an electron withdrawing group. In some embodiments, R⁸ and R⁹ are independently hydrogen or NO₂. In some embodiments, R⁸ is hydrogen. In some embodiments, R⁸ is NO₂. In some embodiments, R⁹ is hydrogen. In some embodiments, R⁹ is NO₂. In some embodiments, one of R⁸ and R⁹ is NO₂.

In some embodiments, an electron withdrawing group provided herein comprises a haloalkyl, a halogen, an amide, or a carbonyl. In some embodiments, an electron withdrawing group comprises a haloalkyl. In some embodiments, an electron withdrawing group comprises CF₃. In some embodiments, an electron withdrawing group comprises a halogen. In some embodiments, an electron withdrawing group comprises an amide. In some embodiments, an electron withdrawing group comprises a carbonyl. In some embodiments, an electron withdrawing group comprises NO₂. In some embodiments, an electron withdrawing group comprises SO₃H. In some embodiments, an electron withdrawing group comprises NH₃. In some embodiments, an electron withdrawing group comprises CN. In some embodiments, an electron withdrawing group comprises COC. In some embodiments, an electron withdrawing group comprises NR₃. In some embodiments, R comprises an alkyl, such as a C₁-C₆ alkyl.

In some embodiments, R², R^{3′}, and R^{4′} are independently selected from one or more linkers, hydrogen, or null.

In some embodiments, R² and R^{3′} are taken together with the atoms from which they are attached to form an aryl, heteroaryl, cycloalkyl, or heterocycloalkyl, the aryl, heteroaryl, cycloalkyl, or heterocycloalkyl being optionally substituted with one or more click-chemistry moieties (e.g., linker-click chemistry moieties). In some embodiments, R² and R^{3′} are taken together with the atoms from which they are attached to form aryl, optionally substituted with one or more linker-click chemistry moieties. In some embodiments, R² and R^{3′} are taken together with the atoms from which they are attached to form heteroaryl, optionally substituted with one or more linker-click chemistry moieties. In some embodiments, R² and R^{3′} are taken together with the atoms from which they are attached to form cycloalkyl, optionally substituted with one or more linker-click chemistry moieties. In some embodiments, R² and R^{3′} are taken together with the atoms from which they are attached to form heterocycloalkyl, optionally substituted with one or more linker-click chemistry moieties.

In some embodiments, R² is a linker, hydrogen, or null. In some embodiments, R² is a linker, such as a linker described elsewhere herein. In some embodiments, R² is hydrogen. In some embodiments, R² is null.

In some embodiments, R^{3′} is a linker, hydrogen, or null. In some embodiments, R^{3′} is a linker, such as a linker described elsewhere herein. In some embodiments, R^{3′} is hydrogen. In some embodiments, R^{3′} is null.

In some embodiments, R⁴′ is a linker, hydrogen, or null. In some embodiments, R^{4′} is a linker, such as a linker described elsewhere herein. In some embodiments, R⁴′is hydrogen. In some embodiments, R⁴′ is null.

In some embodiments, the linker is a cleavable linker, such as a cleavable linker described elsewhere herein.

In some embodiments, R⁵, R⁶, and R⁷ are independently a click-chemistry moiety or null. In some embodiments, R⁵ is a click-chemistry moiety or null. In some embodiments, R⁵ is a click-chemistry moiety. In some embodiments, R⁵ is null. In some embodiments, R⁶ is a click-chemistry moiety or null. In some embodiments, R⁶ is a click-chemistry moiety. In some embodiments, R⁶ is null. In some embodiments, R⁷ is a click-chemistry moiety or null. In some embodiments, R⁷ is a click-chemistry moiety. In some embodiments, R⁷ is null. In preferred embodiments, at least one of R⁵, R⁶, and R⁷ comprise a click-chemistry moiety. In some embodiments, a click-chemistry moiety is a click-chemistry moiety as described elsewhere herein. In some embodiments, the click-chemistry moiety is azide or alkyne.

In some embodiments, R² and R⁵ are null.

In some embodiments, [symbol] is a single or a double bond. In some embodiments, [symbol] is a single bond. In some embodiments, [symbol] is a double bond.

In some embodiments, the sequencing T reagent of Formula IV has the structure of Formula IV′ or a stereoisomer, tautomer, or salt thereof:

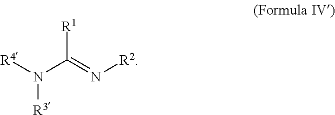

In some embodiments, R¹ comprises one or more leaving groups. In some embodiments, the leaving group is an electrophilic group. In some embodiments, the electrophilic group is substituted or unsubstituted C₃₋₁₀ aryl or substituted or unsubstituted C₃₋₁₀ heteroaryl. In some embodiments, the electrophilic group is substituted or unsubstituted pyrazole or triazole. In some embodiments, the electrophilic group is substituted or unsubstituted pyrazole. In some embodiments, the electrophilic group is substituted or unsubstituted triazole. In some embodiments, the leaving group is:

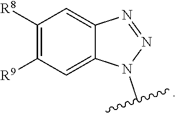

In some embodiments, R² and R^{3′} are taken together with the atoms from which they are attached to form an aryl, heteroaryl, cycloalkyl, or heterocycloalkyl, the aryl, heteroaryl, cycloalkyl, or heteroalkyl being substituted with one or more linker-click chemistry moieties. In some embodiments, R² and R^{3′} are taken together with the atoms from which they are attached to form an aryl, optionally substituted with one or more linker-click chemistry moieties (e.g., CH₂-azide). In some embodiments, R² and R^{3′} are taken together with the atoms from which they are attached to form heteroaryl, optionally substituted with one or more linker-click chemistry moieties (e.g., CH₂-azide). In some embodiments, R² and R^{3′} are taken together with the atoms from which they are attached to form an cycloalkyl, optionally substituted with one or more linker-click chemistry moieties (e.g., CH₂-azide). In some embodiments, R² and R^{3′} are taken together with the atoms from which they are attached to form heterocycloalkyl, optionally substituted with one or more linker-click chemistry moieties (e.g., CH₂-azide).

In some embodiments, R^{4′} is hydrogen. In some embodiments, R^{4′} is C₁₋₆ alkyl.

In some embodiments, the sequencing reagent has the formula V-C, or a stereoisomer, tautomer, or salt thereof:

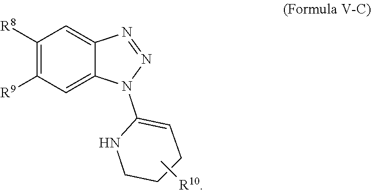

In some embodiments, R⁸ and R⁹ are independently selected from hydrogen or an electron withdrawing group. In some embodiments, R⁸ is hydrogen. In some embodiments, R⁹ is hydrogen. In some embodiments, R⁸ is an electron withdrawing group. In some embodiments, R⁹ is an electron withdrawing group. In some embodiments, R⁸ and R⁹ are independently hydrogen or NO₂. In some embodiments, R⁸ is hydrogen. In some embodiments, R⁸ is NO₂. In some embodiments, R⁹ is hydrogen. In some embodiments, R⁹ is NO₂. In some embodiments, one of R⁸ and R⁹ is NO₂.

In some embodiments, R¹⁰ is a linker-click chemistry moiety.

As used herein, a “linker-click chemistry moiety” may refer to a linker substituted with a click chemistry moiety. In some embodiments, R¹⁰ is CH₂-azide (e.g., CH₂—N₃). In some instances, in a linker-click chemistry moiety, the linker comprises any linker such as described elsewhere herein. In some instances, the linker is a substituted or unsubstituted C₁₋₆ alkyl. In some instances, the linker is an unsubstituted C₁₋₆ alkyl. In some instances, the click chemistry moiety is any click chemistry moiety as described elsewhere herein. In some instances, the click chemistry moiety is an azide or alkyne. In some instance, the linker-click chemistry moiety is C₁₋₆ alkyl substituted with a azide.

In some embodiments, R⁸ is hydrogen, R⁹ is NO₂, and R¹⁰ is CH₂—N₃.

In some embodiments, the sequencing reagent of Formula V has the structure of Formula IV″, or a stereoisomer, tautomer, or salt thereof:

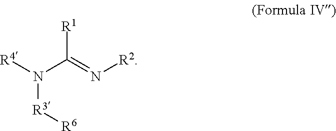

In some embodiments, R¹ comprises one or more leaving groups. In some embodiments, the leaving group is an electrophilic group. In some embodiments, the electrophilic group is substituted or unsubstituted C₃₋₁₀ aryl or substituted or unsubstituted C₃₋₁₀ heteroaryl. In some embodiments, the electrophilic group is substituted or unsubstituted pyrazole or triazole. In some embodiments, the electrophilic group is substituted or unsubstituted pyrazole. In some embodiments, the electrophilic group is substituted or unsubstituted triazole. In some embodiments, the leaving group is:

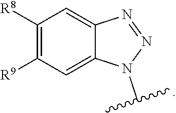

In some embodiments, R² and R^{3′} are independently selected from a linker, hydrogen, or null. In some embodiments, R² is a linker. In some embodiments, R² is hydrogen. In some embodiments, R² is null. In some embodiments, R^{3′} is a linker. In some embodiments, R^{3′} is hydrogen. In some embodiments, R^{3′} is null.

In some embodiments, R^{4′} is substituted or unsubstituted C₁₋₆ alkyl, substituted or unsubstituted C₁₋₆ heteroalkyl, substituted or unsubstituted C₁₋₆ alkoxy, hydrogen, or null. In some embodiments, R^{4′} is substituted or unsubstituted C₁₋₆ alkyl. In some embodiments, R^{4′} is substituted or unsubstituted C₁₋₆ heteroalkyl. In some embodiments, R^{4′} is substituted or unsubstituted C₁₋₆ alkoxy. In some embodiments, R^{4′} is hydrogen. In some embodiments, R^{4′} is null.

In some embodiments, R⁶ is present. In some embodiments, R⁶ is null. In some embodiments, R⁶, if present, is a click chemistry moiety.

In some embodiments, R¹ is

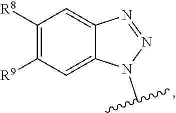

R⁸ is hydrogen, R⁹ is NO₂, R² is hydrogen, R^{3′} is C₁-C₆ alkyl, R⁶ is N₃, and R⁴′ is C₁ alkyl. In some embodiments, R¹ is R¹ is

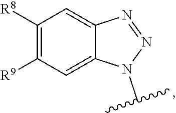

R⁸ is hydrogen, R⁹ is hydrogen, R² is hydrogen, R^{3′} is C₁-C₆ alkyl, R⁶ is N₃, and R^{4′} is C₁ alkyl.

In some embodiments, the sequencing reagent represented by Formula IV is Formula IV-A:

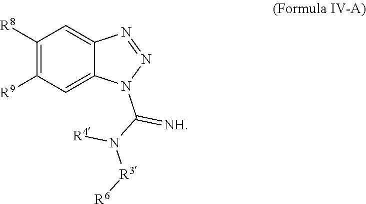

In some embodiments, R^{3′} comprises a linker, such as a linker described elsewhere herein. In some embodiments, R^{3′} is alkyl. In some embodiments, R^{3′} is C₁-C₆ alkyl.

In some embodiments, R⁶ comprises a click chemistry moiety, such as a click chemistry moiety described elsewhere herein. In some embodiments, R⁶ comprises an azide. In some embodiments, R⁶ comprises an alkyne.

In some embodiments, R⁴4′ is hydrogen or alkyl. In some embodiments, R⁴′ is hydrogen. In some embodiments R^{4′} is alkyl, such as C₁-C₆ alkyl. In some embodiments, R^{4′} is heteroalkyl, such a C₁-C₆ heteroalkyl. In some embodiments, R^{4′} is substituted alkyl, such as C₁-C₆ substituted alkyl. In some embodiments, R^{4′} is CH₂CH₂CH₃. In some embodiments, R⁴′ is propyl.

In some embodiments, R⁸ and R⁹ are independently selected from hydrogen or an electron withdrawing group. In some embodiments, R⁸ is hydrogen. In some embodiments, R⁹ is hydrogen. In some embodiments, R⁸ is an electron withdrawing group, such as an electron withdrawing group described elsewhere herein. In some embodiments, R⁹ is an electron withdrawing group, such as an electron withdrawing group described elsewhere herein.

In some embodiments, the sequencing reagent of Formula IV-A is:

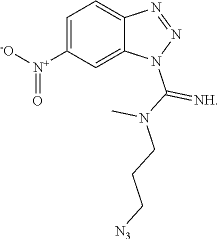

In some embodiments, the sequencing reagent represented by Formula IV is Formula IV-B:

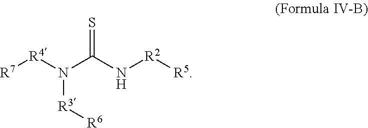

In some embodiments, R², R^{3′}, and R^{4′} are independently selected from hydrogen, one or more linkers, or null. In some embodiments, R² is hydrogen. In some embodiments, R² is a linker, such as a linker described elsewhere herein. In some embodiments, R² is null. In some embodiments, the linker is a cleavable linker, such as a cleavable linker described elsewhere herein. In some embodiments, R² is an alkyl.

In some embodiments, R^{3′} is hydrogen. In some embodiments, R^{3′} is a linker, such as a linker described elsewhere herein. In some embodiments, R^{3′} is null. In some embodiments, the linker is a cleavable linker, such as a cleavable linker described elsewhere herein.

In some embodiments R^{4′} is hydrogen. In some embodiments, R^{4′} is a linker, such as a linker described elsewhere herein. In some embodiments, R⁴′ is null. In some embodiments, the linker is a cleavable linker, such as a cleavable linker described elsewhere herein.

In some embodiments, R⁵, R⁶, and R⁷ are independently click-chemistry moieties or null. In some embodiments, R⁵ is a click-chemistry moiety, such as a click-chemistry moiety described elsewhere herein. In some embodiments, R⁵ is null. In some embodiments, R⁵ is an azide.

In some embodiments, R⁶ is a click-chemistry moiety, such as a click chemistry moiety described elsewhere herein. In some embodiments, R⁶ is null.

In some embodiments, R⁷ is a click chemistry moiety, such as a click chemistry moiety described elsewhere herein. In some embodiments, R⁷ is null.

In some embodiments, R^{3′} and R⁵ are hydrogen.

In some embodiments, the sequencing reagent represented by Formula IV-B is:

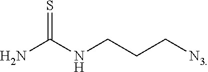

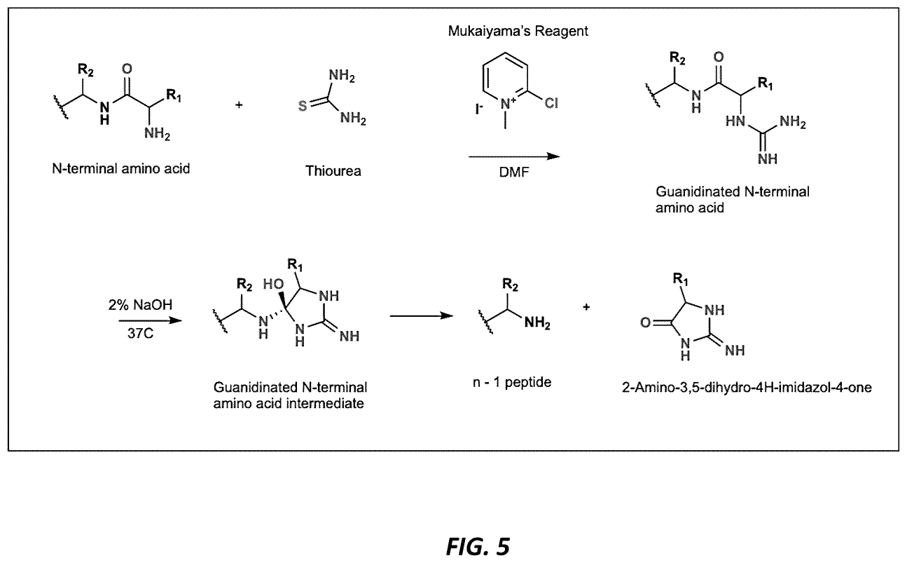

In some embodiments, the sequencing reagent represented by any one of Formula I, IV, IV′, IV″, IV-A, IV-B, or V-C, is selected from Table 1. In some embodiments, the sequencing reagents in Table 1 are sequencing reagents of Formula I. In some embodiments, provided herein sequencing reagents described in FIG. 5-11 or FIG. 13.

TABLE 1

| Sequencing Reagent | Structure |
|---|---|
| 1 | 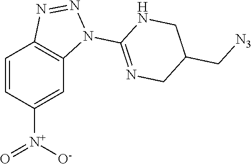 [SMILES: [N-]=[N+]=NCC1CN=C(n2nnc3ccc([N+](=O)[O-])cc32)NC1] |
| 2 | 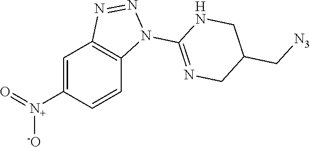 |
| 3 | 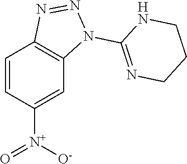 [SMILES: O=[N+]([O-])c1ccc2nnn(C3=NCCCN3)c2c1] |
| 4 | 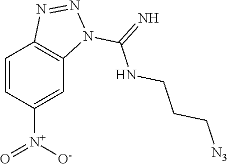 [SMILES: [N-]=[N+]=NCCCNC(=N)n1nnc2ccc([N+](=O)[O-])cc21] |
| 5 | 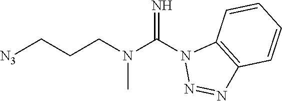 [SMILES: CN(CCCN=[N+]=[N-])C(=N)n1nnc2ccccc21] |
| 6 | 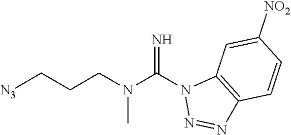 [SMILES: CN(CCCN=[N+]=[N-])C(=N)n1nnc2ccc([N+](=O)[O-])cc21] |
| 7 | 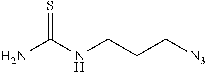 |

### Reactive Group

In some embodiments, the reactive group provided herein is configured to form a covalent bond with a monomer of a polymeric analyte. In some embodiments, the monomer is an amino acid of a peptide. In some instances, the amino acid is an N-terminal amino acid of the peptide. In some embodiments, the reactive group comprises a guanidinylating agent. In some instances, a guanidinylating agent is a moiety that adds a guanidine to a molecule.

In some embodiments, A comprises a compound of Formula (I-A):

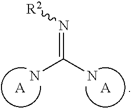

In some embodiments, R² is H, R⁴, OH, OR⁴, NH₂, or —NHR⁴. In some embodiments, R² is H. In some embodiments, R² is R⁴. In some embodiments, R² is OH. In some embodiments, R² is OR⁴. In some embodiments, R² is NH₂. In some embodiments, R² is —NHR⁴.

In some embodiments, R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂, where each R″ is independently H or C₁₋₃ alkyl. In some embodiments, R⁴ is C₁₋₆ alkyl. In some embodiments, R⁴ is C₁₋₆ alkyl optionally substituted with one or two members selected from halo, C₁₋₃ alkyl, C₁₋₃ alkoxy, C₁₋₃ haloalkyl, phenyl, 5-membered heteroaryl, and 6-membered heteroaryl. In some embodiments, R⁴ is C₁₋₆ alkyl substituted with halo. In some embodiments, R⁴ is C₁₋₆ alkyl substituted with C₁₋₃ alkyl. In some embodiments, R⁴ is C₁₋₆ alkyl substituted with C₁₋₃ alkoxy. In some embodiments, R⁴ is C₁₋₆ alkyl substituted with C₁₋₃ haloalkyl. In some embodiments, R⁴ is C₁₋₆ alkyl substituted with phenyl. In some embodiments, R⁴ is C₁₋₆ alkyl substituted with 5-membered heteroaryl. In some embodiments, R⁴ is C₁₋₆ alkyl substituted with 6-membered heteroaryl.

In some embodiments, the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂. In some embodiments, the phenyl, 5-membered heteroaryl, and 6-membered heteroaryl is substituted with halo. In some embodiments, the phenyl, 5-membered heteroaryl, and 6-membered heteroaryl is substituted with —OH. In some embodiments, the phenyl, 5-membered heteroaryl, and 6-membered heteroaryl is substituted with C₁₋₃ alkyl. In some embodiments, the phenyl, 5-membered heteroaryl, and 6-membered heteroaryl is substituted with C₁₋₃ alkoxy. In some embodiments, the phenyl, 5-membered heteroaryl, and 6-membered heteroaryl is substituted with C₁₋₃ haloalkyl. In some embodiments, the phenyl, 5-membered heteroaryl, and 6-membered heteroaryl is substituted with NO₂. In some embodiments, the phenyl, 5-membered heteroaryl, and 6-membered heteroaryl is substituted with CN. In some embodiments, the phenyl, 5-membered heteroaryl, and 6-membered heteroaryl is substituted with COOR″. In some embodiments, the phenyl, 5-membered heteroaryl, and 6-membered heteroaryl is substituted with CON(R″)₂.

In some embodiments, each R″ is independently H or C₁₋₃ alkyl. In some embodiment, R″ is H. In some embodiments, R″ is C₁₋₃ alkyl.

In some embodiments, ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl. In some embodiments, ring A is optionally fused to an additional phenyl. In some embodiments, ring A is optionally fused to a 5-6 membered heteroaryl. In some embodiments, the 5-membered heteroaryl ring is optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl. In some embodiments, the optional fused phenyl is optionally substituted with optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl. In some embodiments, the optional 5-6 membered heteroaryl ring is optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl.

In some embodiments, each R is independently selected from H and C₁₋₃ alkyl optionally substituted with OH, OR*, —NH₂, —NHR*, or —NR*₂. In some embodiments, each R is H. In some embodiments, each R is C₁₋₃ alkyl. In some embodiments, each R is C₁₋₃ alkyl optionally substituted with OH, OR*, —NH₂, —NHR*, or —NR*₂. In some embodiments, each R is C₁₋₃ alkyl optionally substituted with OR*. In some embodiments, each R is C₁₋₃ alkyl optionally substituted with —NH₂. In some embodiments, each R is C₁₋₃ alkyl optionally substituted with —NHR*. In some embodiments, each R is C₁₋₃ alkyl optionally substituted with —NR*₂.

In some embodiments, each R* is C₁₋₃ alkyl, optionally substituted with OH, oxo, C₁₋₂ alkoxy, or CN. In some embodiments, each R* is C₁₋₃ alkyl. In some embodiments, each R* is C₁₋₃ alkyl optionally substituted with OH. In some embodiments, each R* is C₁₋₃ alkyl optionally substituted with oxo. In some embodiments, each R* is C₁₋₃ alkyl optionally substituted with C₁₋₂ alkoxy. In some embodiments, each R* is C₁₋₃ alkyl optionally substituted with CN.

In some embodiments, two R, or two R″, or two R* on the same N can be optionally taken together to form a 4-7 membered heterocyclic ring, optionally containing an additional heteroatom selected from N, O, and S as a ring member, and optionally substituted with one or two groups selected from halo C₁₋₂ alkyl, OH, oxo, C₁₋₂ alkoxy, and CN. In some embodiments, two R can be optionally taken together to form a 4-7 membered heterocyclic ring. In some embodiments, two R″ can be optionally taken together to form a 4-7 membered heterocyclic ring. In some embodiments, two R* can be taken together to form a 4-7 membered heterocyclic ring. In some embodiments, the 4-7 membered heterocyclic ring optionally contains an additional heteroatom. In some embodiments, the 4-7 membered heterocyclic ring optionally contains an additional heteroatom selected from N, O, and S as a ring member. In some embodiments, the 4-7 membered heterocyclic ring optionally contains N as a ring member. In some embodiments, the 4-7 membered heterocyclic ring optionally contains O as a ring member. In some embodiments, the 4-7 membered heterocyclic ring optionally contains S as a ring member. In some embodiments, the 4-7 membered heterocyclic ring is optionally substituted with one or two groups selected from halo, C₁₋₂ alkyl, OH, oxo, C₁₋₂ alkoxy, and CN. In some embodiments, the 4-7 membered heterocyclic ring is substituted with halo. In some embodiments, the 4-7 membered heterocyclic ring is substituted with C₁₋₂ alkyl. In some embodiments, the 4-7 membered heterocyclic ring is substituted with OH. In some embodiments, the 4-7 membered heterocyclic ring is substituted with oxo. In some embodiments, the 4-7 membered heterocyclic ring is substituted with C₁₋₂ alkoxy. In some embodiments, the 4-7 membered heterocyclic ring is substituted with CN.

In some embodiments, the reactive group (e.g., A) comprises a structure of Formula I-B, or a stereoisomer, tautomer, or salt thereof.

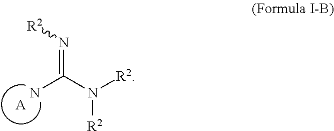

In some embodiments, each R² is independently hydrogen, R⁴, OH, OR⁴, NH₂, or —NHR⁴. In some embodiments, R² is hydrogen. In some embodiments, R² is R⁴. In some embodiments, R² is OH. In some embodiments, R² is OR⁴. In some embodiments, R² is NH₂. In some embodiments, R² is —NHR⁴. In some embodiments, each R² are independently hydrogen or R⁴.

In some embodiments, R⁴ is C₁₋₆ alkyl or C₁₋₆ alkoxy, each of which is optionally substituted. In some embodiments, R⁴ is optionally substituted C₁₋₆ alkyl. In some embodiments, R⁴ is optionally substituted C₁₋₆ alkoxy. In some embodiments, R⁴ is C₁₋₆ alkyl optionally substituted with halo, C₁₋₆ alkyl, C₁₋₆ alkoxy, C₁₋₆ haloalkyl, oxo, aryl, and 5-6-membered heteroaryl.

In some embodiments, R⁴ is C₁₋₆ alkyl optionally substituted with halo. In some embodiments, R⁴ is C₁₋₆ alkyl optionally substituted with C₁₋₆ alkyl. In some embodiments, R⁴ is C₁₋₆ alkyl optionally substituted with C₁₋₆ alkoxy. In some embodiments, R⁴ is C₁₋₆ alkyl optionally substituted with C₁₋₆ haloalkyl. In some embodiments, R⁴ is C₁₋₆ alkyl optionally substituted with oxo. In some embodiments, R⁴ is C₁₋₆ alkyl optionally substituted with aryl. In some embodiments, R⁴ is C₁₋₆ alkyl optionally substituted with 5-6 membered heteroaryl. In some embodiments, the aryl and heteroaryl are optionally substituted. In some embodiments, the aryl and heteroaryl are optionally substituted with one or two members selected from halo, —OH, C₁₋₄ alkyl, C₁₋₄ alkoxy, C₁₋₄ haloalkyl, NO₂, CN, COOR″, and CON(R″)₂, where each R″ is independently H or C₁₋₃ alkyl.

In some embodiments, R⁴ is C₁₋₆ alkoxy optionally substituted with halo. In some embodiments, R⁴ is C₁₋₆ alkoxy optionally substituted with C₁₋₆ alkyl. In some embodiments, R⁴ is C₁₋₆ alkoxy optionally substituted with C₁₋₆ alkoxy. In some embodiments, R⁴ is C₁₋₆ alkoxy optionally substituted with C₁₋₆ haloalkyl. In some embodiments, R⁴ is C₁₋₆ alkoxy optionally substituted with oxo. In some embodiments, R⁴ is C₁₋₆ alkoxy optionally substituted with aryl. In some embodiments, R⁴ is C₁₋₆ alkoxy optionally substituted with 5-6 membered heteroaryl. In some embodiments, the aryl and heteroaryl are optionally substituted. In some embodiments, the aryl and heteroaryl are optionally substituted with one or two members selected from halo, —OH, C₁₋₄ alkyl, C₁₋₄ alkoxy, C₁₋₄ haloalkyl, NO₂, CN, COOR″, and CON(R″)₂, where each R″ is independently H or C₁₋₃ alkyl.

In some embodiments, R⁴ is C₁₋₆ alkyl or C₁₋₆ alkoxy, each optionally substituted with one or more of C₁₋₆ alkyl, oxo, and aryl.

In some embodiments, Ring A is a 5-6 membered heteroaryl ring. In some embodiments, Ring A is a 5-6 membered heteroaryl ring comprising up to three (e.g., 1, 2, or 3) N atoms as ring members. In some embodiments, the 5-6 membered heteroaryl ring is optionally fused to an additional aryl or 5-6 membered heteroaryl ring. In some embodiments, Ring A is an optionally substituted C₅-12 heteroaryl. In some embodiments, the aryl is optionally substituted with one or two groups selected from C₁₋₄ alkyl, C₁₋₄ alkoxy, —OH, halo, C₁₋₄ haloalkyl, NO₂, COOR, CONR₂, —SO₂R^{a}, —NR₂, phenyl, and 5-6 membered heteroaryl. In some embodiments, Ring A is substituted with one or more electron withdrawing groups. In some embodiments, Ring A is substituted with C₁₋₄ alkyl. In some embodiments, Ring A is substituted with C₁₋₄ alkoxy. In some embodiments, Ring A is substituted with —OH. In some embodiments, Ring A is substituted with halo. In some embodiments, Ring A is substituted with C₁₋₄ haloalkyl. In some embodiments, Ring A is substituted with NO₂. In some embodiments, Ring A is substituted with COOR. In some embodiments, Ring A is substituted with CONR₂. In some embodiments, Ring A is substituted with —SO₂R^{a}. In some embodiments, Ring A is substituted with —NR₂. In some embodiments, Ring A is substituted with phenyl. In some embodiments, Ring A is substituted with 5-6 membered heteroaryl.

In some embodiments, Ring A is (e.g., optionally substituted) pyrazole, triazole, or

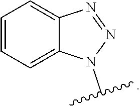 [SMILES: *n1nnc2ccccc21]

In some embodiments, Ring A is (e.g., optionally substituted) pyrazole. In some embodiments, Ring A is (e.g., optionally substituted) triazole. In some embodiments, Ring A is (e.g., optionally substituted)

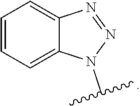 [SMILES: *n1nnc2ccccc21]

In some embodiments, R is independently selected from hydrogen and optionally substituted C₁₋₃ alkyl. In some embodiments, R is C₁₋₃ alkyl optionally substituted with OH, OR^{a}, —NH₂, —NHR^{a}, or —NR^{a}₂. In some embodiments, R is C₁₋₃ alkyl optionally substituted with OH. In some embodiments, R is C₁₋₃ alkyl optionally substituted with OR^{a}. In some embodiments, R is C₁₋₃ alkyl optionally substituted with NH₂. In some embodiments, R is C₁₋₃ alkyl optionally substituted with —NHR^{a}. In some embodiments, R is C₁₋₃ alkyl optionally substituted with —NR^{a}₂.

In some embodiments, each R^{a} is C₁₋₃ alkyl, optionally substituted with OH, oxo, C₁₋₂ alkoxy, or CN. In some embodiments, R^{a} is C₁₋₃ alkyl. In some embodiments, each R^{a} is C₁₋₃ alkyl, optionally substituted with OH. In some embodiments, each R^{a} is C₁₋₃ alkyl, optionally substituted with oxo. In some embodiments, each R^{a} is C₁₋₃ alkyl, optionally substituted with C₁₋₂ alkoxy. In some embodiments, each R^{a} is C₁₋₃ alkyl, optionally substituted with CN.

In some instances, two R, two R″, or two R^{a} on the same N can optionally be taken together to form a 4-7 membered heterocyclic ring. In some embodiments, the 4-7 membered heterocylic ring optionally contains a heteroatom selected from N, O, and S as a ring member. In some embodiments, the 4-7 membered ring is optionally substituted with one or two groups selected from halo, C₁₋₂ alkyl, OH, oxo, C₁₋₂ alkoxy, and CN. In some embodiments, the 4-7 membered ring is optionally substituted with halo. In some embodiments, the 4-7 membered ring is optionally substituted with C₁₋₂ alkyl. In some embodiments, the 4-7 membered ring is optionally substituted with C₁₋₂ alkoxy. In some embodiments, the 4-7 membered ring is optionally substituted with CN.

In some embodiments, the reactive group comprises:

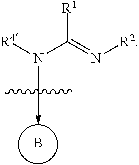

In some embodiments, R¹ comprises one or more leaving groups. In some embodiments, R¹ is a leaving group.

In some embodiments, R² is a linker, hydrogen, or null. In some embodiments, R² is a linker. In some embodiments, R² is hydrogen. In some embodiments, R² is null.

In some embodiments, R⁴′ is substituted or unsubstituted C₁₋₆ alkyl, substituted or unsubstituted C₁₋₆ heteroalkyl, substituted or unsubstituted C₁₋₆ alkoxy, hydrogen, or null. In some embodiments, R⁴′ is substituted or unsubstituted C₁₋₆ alkyl. In some embodiments, R^{4′} is substituted or unsubstituted C₁₋₆ heteroalkyl. In some embodiments, R⁴′ is substituted or unsubstituted C₁₋₆ alkoxy. In some embodiments, R^{4′} is hydrogen. In some embodiments, R⁴′ is null.

In some embodiments, the reactive group comprises:

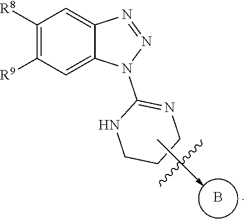

In some embodiments, R⁸ and R⁹ are independently hydrogen or NO₂. In some embodiments, R⁸ is hydrogen. In some embodiments, R⁸ is NO₂. In some embodiments, R⁹ is hydrogen. In some embodiments, R⁹ is NO₂. In some embodiments, one of R⁸ and R⁹ is NO₂. In other embodiments, R⁸ and R⁹ are independently selected from hydrogen or an electron withdrawing group. In some embodiments, R⁸ is an electron withdrawing group. In some embodiments, R⁹ is an electron withdrawing group.

In some embodiments, the reactive group comprises:

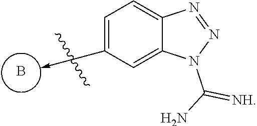

In some embodiments, the reactive group comprises:

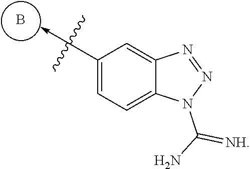

In some embodiments, the reactive group comprises:

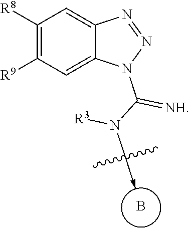

In some embodiments, R⁸ and R⁹ are independently hydrogen or NO₂. In some embodiments, R⁸ is hydrogen. In some embodiments, R⁸ is NO₂. In some embodiments, R⁹ is hydrogen. In some embodiments, R⁹ is NO₂. In some embodiments, one of R⁸ and R⁹ is NO₂.

In some embodiments, the reactive group comprises:

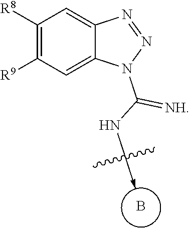

In some instances,

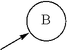

indicates orientation of the reactive group relative to the capture-binding moiety.

In some embodiments, the reactive group comprises Formula II-A′:

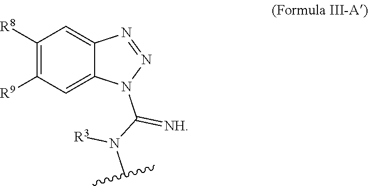

In some embodiments, R³ is hydrogen or substituted or unsubstituted C₁₋₆ alkyl. In some embodiments, R³ is hydrogen. In some embodiments, R³ is substituted or unsubstituted C₁₋₆ alkyl.

In some embodiments, R⁸ and R⁹ are independently hydrogen or NO₂. In some embodiments, R⁸ is hydrogen. In some embodiments, R⁸ is NO₂. In some embodiments, R⁹ is hydrogen. In some embodiments, R⁹ is NO₂. In some embodiments, one of R⁸ and R⁹ is NO₂.

In some embodiments, the reactive group comprises Formula II-A′:

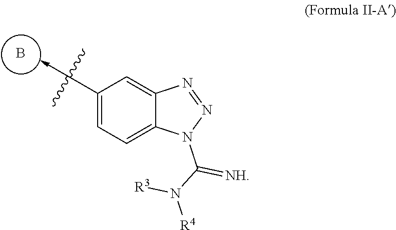

In some embodiments, the reactive group comprises Formula II-B′:

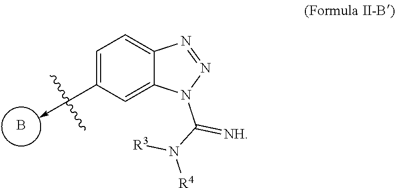

In some embodiments, R⁴ comprises a click-chemistry moiety, such as a click chemistry moiety described elsewhere herein. In some embodiments, R⁴ is an alkyne. In some embodiments, R⁴ is an azide.

In some embodiments, R³ is hydrogen or alkyl, substituted alkyl, or heteroalkyl. In some embodiments, R³ is hydrogen. In some embodiments, R³ is alkyl. In some embodiments, R³ is substituted alkyl. In some embodiments, R³ is heteroalkyl.

In some embodiments,

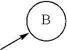

indicates orientation of the reactive group relative to the capture-binding group.

In some embodiments, the reactive group comprises Formula III-A′:

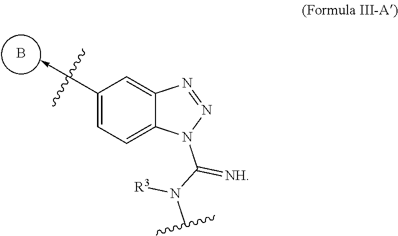

In some embodiments, the reactive group comprises Formula III-B′:

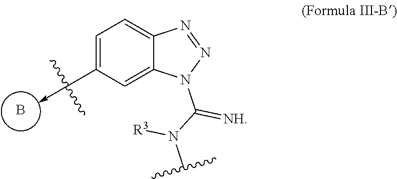

In some embodiments, R³ comprises an alkyl. In some embodiments, R³ comprises an alkane group.

In some embodiments,

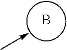

indicates orientation of the reactive group relative to the capture-binding moiety.

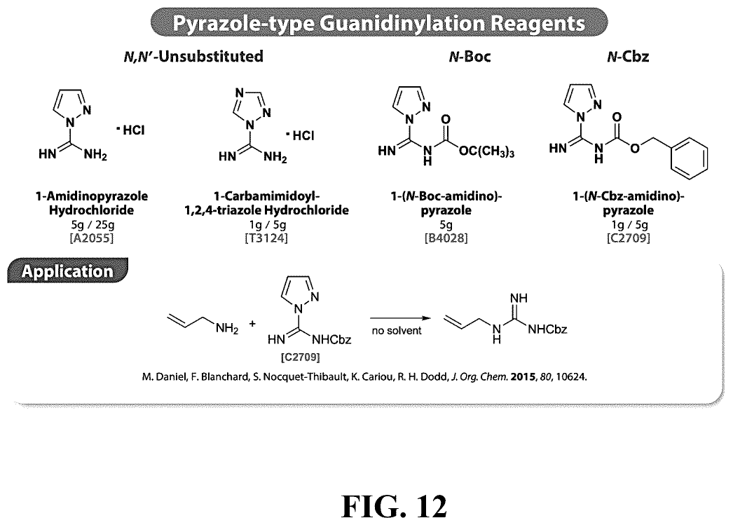

In some instances, a reactive group of any one of Formula I-A, I-B, II-A, or III-A is represented in Table 2. In some embodiments, provided herein are reactive groups such as those described in FIG. 12.

TABLE 2

| Reactive Group | Structure |
|---|---|
| 1 | 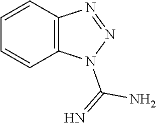 [SMILES: N=C(N)n1nnc2ccccc21] |
| 2 | 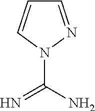 |
| 3 | 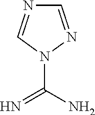 |
| 4 | 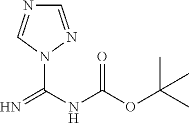 |
| 5 | 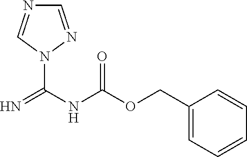 [SMILES: N=C(NC(=O)OCc1ccccc1)n1cncn1] |

In some embodiments, guanylation or guanidinylation is completed in the absence of a catalyst. In some embodiments, guanylation or guanidinylation is completed in the presence of a catalyst. In some embodiments, guanylation or guanidinylation of amines is completed in the presence of a catalyst. In some embodiments, the catalyst comprises nickel, such as a nickel-promoted guanylation or guanidinylation. In some instances, the nickel is Nickel(0). In some embodiments, the catalyst is nickel phosphide, nickel boride, aluminum nickel, nickel on kieselguhr, or nickel on silica/alumina.

### Capture-Binding Moiety

In some embodiments, provided herein are capture-binding moieties that allow for coupling or attachment, such as covalent attachment, of a sequencing reagent provided herein to a capture moiety.

The capture-binding moiety may comprise any useful capture molecule, including but not limited to, chemical linkers (e.g., click chemistry moieties), functional or reactive groups (e.g., succinimidyl (NHS) esters, or other chemical linker, described elsewhere herein), or a polymerizable molecule. A polymerizable molecule may comprise biological polymers (e.g., nucleic acid molecules, peptides, polysaccharides, fatty acids), or other naturally occurring polymers, e.g., rubber, cellulose, starches, polyhydroxyalkanoates, chitosan, dextran, structural proteins (e.g., collagen, hyaluronic acid, glycosaminoglycans), agarose, carrageenan, isphagula, acacia, agar, gelatin, shellac, xanthan gum, guar gum, alginate, etc. The polymerizable molecules may be synthetic, e.g., acrylics, nylons, silicones, viscose, rayon, polyesters, polycarboxylic acids, polyvinyl acetate, polyacrylamide, polyacrylate, polyethylene glycol, polyurethane, polylactic acid, silica, polystyrene, polyacrylonitrile, polybutadiene, polycarbonate, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and combinations thereof. The polymerizable molecules may comprise one or more reactive moieties (e.g., radical groups) to initiate polymerization or may be polymerized via contacting of an initiating agent (e.g., ammonium persulfate, peroxide, or other radicalizing agent). The polymerizable molecules may be polymerizable via contacting of an enzyme (e.g., polymerizing enzyme such as polymerases), ribozyme or DNAzyme. Alternatively or in addition to, the polymerizable molecules may be polymerizable via self-assembly. The polymerizable molecules may comprise a single polymer type (e.g., a homopolymer) or more than one polymer type (e.g., a copolymer) and may comprise random or arranged monomers. The polymerizable molecules may be a block polymer, alternating copolymer, periodic copolymer, statistical copolymer, stereoblock copolymer, gradient copolymer, branched copolymer, graft copolymer, etc.

The capture-binding moiety of the sequencing reagent may be coupled to or capable of coupling, either directly or indirectly, to the capture moiety. The capture-binding moiety and the capture moiety may be coupled using any useful chemistry or interaction, such as the interaction of binding pairs, e.g., biotin (or similar molecule such as desthiobiotin) and avidin (or similar molecule such as neutravidin, streptavidin), antigen or epitope and antibody or antibody fragment, cyclodextrins and small hydrophobic molecules (e.g., alkanes, benzene, polycyclics), cucurbiturils and adamantaneammonium or trimethylammoniomethyl ferrocene, cyclophane (e.g., calixarenes, cavitands, pillararenes, tetralactams), etc. In some embodiments, the coupling of the capture moiety to the capture-binding moiety occurs through coupling of nucleic acid molecules (e.g., hybridization to one another or to a splint molecule, blunt-end ligation, etc.). In some embodiments, the coupling of the capture moiety to the capture-binding moiety occurs via click chemistry. For example, the capture-binding moiety may comprise a first click chemistry moiety that can react with a second click chemistry moiety comprised by the capture moiety.

The coupling of the capture moiety and the capture-binding moiety may be a direct coupling or an indirect coupling. In an example of direct coupling, the capture moiety may comprise a click chemistry moiety (e.g., alkyne), and the capture-binding moiety of the sequencing reagent may comprise an additional click chemistry moiety (e.g., azide) that can react with the click chemistry moiety of the capture moiety. In another example, the capture-binding moiety comprises a first nucleic acid molecule and the capture moiety comprises a second nucleic acid molecule. The first nucleic acid molecule and the second nucleic acid molecule may be capable of coupling via hybridization, ligation, splint hybridization, etc.

Alternatively, the capture-binding moiety may couple indirectly to the capture moiety, e.g., via an intermediate linking molecule. In one such example, the sequencing reagent comprises a first reactive group (e.g., guanidinylating agent) and a capture-binding moiety comprising a second reactive group (e.g., click chemistry moiety), and the capture moiety comprises a nucleic acid molecule. The coupling of the capture-binding moiety and the capture moiety may be mediated by provision of an intermediate linking molecule, such as a click-functionalized nucleic acid molecule, which can link to the capture moiety (via hybridization or ligation, e.g., using a ligating enzyme such as a ligase) and the capture-binding moiety (via click chemistry). In another example, the capture-binding moiety of the sequencing reagent comprises a neutravidin moiety, the capture moiety comprises a streptavidin moiety, and coupling of the capture-binding moiety to the capture moiety can be mediated by a biotin intermediate linking molecule.

The capture moiety may be coupled to the capture-binding moiety via covalent or noncovalent interaction. In an example of noncovalent interaction, the capture-binding moiety may comprise an avidin or streptavidin tag, which can bind to a biotin capture moiety. Alternatively, the capture moiety can be covalently coupled to the capture-binding moiety, e.g., using the chemical conjugation approaches described herein or via protein engineering approaches, e.g., generating a fusion protein capture-binding moiety, SpyTag and SpyCatcher interaction, SNAP-tag, or other attachment or coupling strategy. In such examples, a protein capture-binding moiety may be generated and conjugated (e.g., using a linker on lysine residues) to a sequencing reagent precursor comprising the guanidinylating agent.

In some embodiments, the capture-binding moiety or the capture moiety comprises a nucleic acid molecule, e.g., a DNA, RNA, or DNA:RNA hybrid oligonucleotide. The nucleic acid molecule can comprise any naturally occurring, non-naturally occurring or engineered nucleotide base. For example, the nucleic acid molecule may comprise a pseudo-complementary base, a bridged nucleic acid, a xenonucleic acid, a locked nucleic acid, a peptide nucleic acid (PNA), a gamma-PNA, a morpholino, etc., as is described elsewhere herein. The capture moiety or capture-binding moiety may comprise one or more functional sequences, including, but not limited to a priming sequence, sequencing sequence (e.g., P5 or P7 sequence), sequencing read sequence (e.g., R1 or R2 sequence), a mosaic end sequence, a transposase recognition sequence, a cleavage site (e.g., restriction site), a UMI, a blocking group, a spacer sequence, a barcode sequence, or other functional sequence. In some instances, the capture moiety comprises a cleavable or releasable moiety, e.g., a restriction enzyme recognition site, an abasic site, a uracil which can be cleaved using USER® or uracil DNA glycosylase, a disulfide bond that can be releasable upon addition of a reducing agent, etc.

In some instances, the capture moiety is coupled to a polymeric analyte that is to be analyzed. In one such example, the capture moiety may comprise a nucleic acid molecule coupled to a polymeric analyte. The nucleic acid molecule may be configured to couple to the capture-binding moiety, as is described elsewhere herein.

In some instances, the capture moiety is coupled to a substrate. The capture moiety may be directly coupled or indirectly coupled to the substrate. The capture moiety (e.g., a click chemistry moiety, a polymerizable molecule such as a nucleic acid molecule) may be attached to the substrate using any suitable approach, as described elsewhere herein, and the coupling may be a covalent or noncovalent interaction. The substrate may comprise any useful solid support, e.g., flow cells, beads, microfluidic devices, microscope slides, planar surfaces, or other support. In some instances, the substrate comprises commercially available beads (e.g., DNA beads or barcoded beads), flow cells, or chips, e.g., Illumina® HiSeq, iSeq, MiniSeq, NextSeq, NovaSeq, etc. may be used as the substrates described herein. In some instances, the substrate comprises a plurality of sequencing primer sequences (e.g., P5 or P7 sequences) or read sequences (e.g., R1 or R²), which can be used as capture moieties.

The capture moiety may comprise a substrate-tethering group or linker or additional functional group and may be provided separately or as part of a surface. In some examples, the capture moiety comprises a nucleic acid molecule that comprises a substrate-tethering group, e.g., biotin, a click chemistry moiety such as an azide, that can couple to a substrate, e.g., a substrate comprising streptavidin or a complementary click chemistry moiety that can react with that of the substrate-tethering group. The capture moiety may additionally comprise a binding sequence, to which another nucleic acid molecule (e.g., a linking nucleic acid molecule, a nucleic acid barcode molecule) can couple, e.g., via hybridization, ligation, or both. In some instances, the capture moiety comprises a single-stranded oligonucleotide or a single-stranded region in which a complementary oligonucleotide (e.g., of the capture-binding moiety or intermediate linking molecule) can hybridize. The complementary oligonucleotide may comprise a detectable label (e.g., fluorophore) that allows for detection of the capture moiety.

Alternatively, or in addition to, the capture moiety may be coupled to the polymeric analyte. For example, the capture moiety may comprise a nucleic acid molecule that is attached to a peptide analyte. The capture moiety may comprise a nucleic acid barcode molecule, which can comprise identifying information on the peptide analyte, e.g., the sample, partition, location, etc. from which the peptide analyte originated.

In some embodiments, the capture-binding moiety comprises iodoacetamide, comprising the structure:

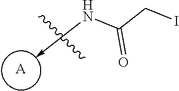

wherein

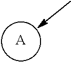

indicates orientation of the capture-binding moiety relative to the reactive group.

In some embodiments, the capture-binding moiety is covalently linked to a polymer. For example, the sequencing reagent may comprise the reactive group, the polymer, and the capture-binding moiety. Alternatively or in addition to, the sequencing reagent may comprise the reactive group and a first click chemistry group (e.g., azide) that can react with a linking molecule, such as a polymer, comprising a second click chemistry group (e.g., alkyne, such as DBCO). The linking molecule can additionally comprise a capture-binding moiety. In such examples, reaction of the first click chemistry group and the second click chemistry group may result in a product that comprises (i) a reactive group, (ii) the linker (or linking molecule), and (iii) a capture-binding moiety.

Alternatively or in addition to, in some embodiments, the capture-binding moiety may comprise a polymer. In some instances, the polymer is a nucleic acid molecule (e.g., DNA or RNA). In an example, the capture-binding moiety comprises a nucleic acid molecule comprising a first nucleic acid sequence, and the capture moiety comprises a second nucleic acid molecule comprising a second nucleic acid sequence. The first nucleic acid sequence may be complementary or partially complementary to and hybridize to the second nucleic acid sequence, or alternatively, a bridge or splint oligonucleotide may be used to hybridize to the first nucleic acid sequence and the second nucleic acid sequence, thereby coupling the capture-binding moiety to the capture moiety. The first nucleic acid sequence and the second nucleic acid sequence may be covalently linked (e.g., via ligation). In some instances, the first nucleic acid sequence may comprise a click chemistry moiety that can react and covalently link to an additional terminal click chemistry moiety of the second nucleic acid sequence.

The polymer can be a synthetic polymer, such as a polyalkylene glycol (PAG) (e.g., polyethylene glycol (PEG) or polypropylene glycol (PPG), or a naturally-occurring polymer (which can be synthetic) such as deoxyribonucleic acid (DNA) or ribonucleic acid (RNA). In some embodiments, the polymer comprises polyethylene glycol (PEG). In some embodiments, the polymer comprises polypropylene glycol (PPG).

In some embodiments, the polymer comprises deoxyribonucleic acid (DNA) or ribonucleic acid (RNA). In some instances, deoxyribonucleic acid (DNA) is attached to a reactive group provided herein to provide a sequencing reagent. In some embodiments, DNA comprising a click chemistry moiety, such as an azide or alkyne, is attached to a reactive group comprising a click-chemistry moiety, such as an azide or an alkyne. For instance, a DBCO-conjugated DNA molecule may be conjugated to an azide-comprising reactive group. In other examples, the click chemistry moiety of the reactive group or the polymer may comprise BCN, tetrazine, TCO, triazole, or other click chemistry moiety, as described elsewhere herein.

In some embodiments, the polymer is configured to link to or is covalently linked to a substrate. In other embodiments, the capture-binding moiety is configured to link to or is covalently linked to a surface-bound linker. In some embodiments, the surface-bound linker is linked to a surface. In some embodiments, the substrate comprises a thiol group or an acrylate group.

In some embodiments, the polymer, such as PEG, comprises between 1 and 20 monomers. In some embodiments, the polymer comprises 1 monomer to 500 monomers. In some embodiments, the polymer comprises 1 monomer to 20 monomers, 1 monomer to 50 monomers, 1 monomer to 100 monomers, 1 monomer to 200 monomers, 1 monomer to 300 monomers, 1 monomer to 400 monomers, 1 monomer to 500 monomers, 20 monomers to 50 monomers, 20 monomers to 100 monomers, 20 monomers to 200 monomers, 20 monomers to 300 monomers, 20 monomers to 400 monomers, 20 monomers to 500 monomers, 50 monomers to 100 monomers, 50 monomers to 200 monomers, 50 monomers to 300 monomers, 50 monomers to 400 monomers, 50 monomers to 500 monomers, 100 monomers to 200 monomers, 100 monomers to 300 monomers, 100 monomers to 400 monomers, 100 monomers to 500 monomers, 200 monomers to 300 monomers, 200 monomers to 400 monomers, 200 monomers to 500 monomers, 300 monomers to 400 monomers, 300 monomers to 500 monomers, or 400 monomers to 500 monomers. In some embodiments, the polymer comprises 1 monomer, 20 monomers, 50 monomers, 100 monomers, 200 monomers, 300 monomers, 400 monomers, or 500 monomers. In some embodiments, the polymer comprises at least 1 monomer, 20 monomers, 50 monomers, 100 monomers, 200 monomers, 300 monomers, or 400 monomers. In some embodiments, the polymer comprises at most 20 monomers, 50 monomers, 100 monomers, 200 monomers, 300 monomers, 400 monomers, or 500 monomers.

In some embodiments, the capture-binding moiety comprises:

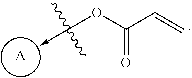

In some instances,

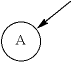

indicates orientation of the capture-binding moiety relative to the reactive group.

In other embodiments, the capture-binding moiety comprises a thiol group. In some embodiments, the capture moiety comprises an acrylate group. In some embodiments, the capture-binding moiety comprises a thiol group and the capture moiety comprises an acrylate group.

### Linker

In some embodiments, the sequencing reagents or reactive groups provided herein comprise a linker (e.g., L¹). The linker of Lⁱ may comprise any useful moiety, which can be used to link the reactive group with the capture-binding moiety. The linker may comprise may comprise an alkyl chain (e.g., methyl, ethyl, propyl, butyl, etc.).In some embodiments, the linker can be a synthetic polymer, such as a polyalkylene glycol (PAG) (e.g., polyethylene glycol (PEG) or polypropylene glycol (PPG), polyacrylamide, or a naturally-occurring polymer (which can be synthetic) such as deoxyribonucleic acid (DNA) or ribonucleic acid (RNA). The linker may additionally or alternatively comprise any number of spacing moieties, e.g., polymers (e.g., PEG, PVA, polyacrylamide), aminohexanoic acid, nucleic acids, etc. Such spacing moieties may be useful in modulating the distance between the reactive group and the capture-binding group.

In some instances, the linker of Li comprises a polymer. For example, the sequencing reagent may comprise the reactive group, the polymer, and the capture-binding moiety. Use of a polymer as a linker can be advantageous in, for example, modulating the atomic distance between the reactive group and the capture-binding moiety, which may be useful in controlling reaction kinetics, reactivity, or other parameter. In some embodiments, Li comprises a hydrophilic polymer.

In some embodiments, the linker (e.g., Li) comprises a polyalkylene glycol linker. The linker may be, for example, a linear or branched polyalkylene glycol (PAG). For example, the linker may be a branched PEG linker. The branched PEG linker may have 3 arms or 4 arms. In some embodiments, the linker has a general structure of PEG CCO—CCO—CCO or PPG CCCO—CCCO—CCCO. In some embodiments, the linker is a Poly(propylene oxide) linker, such as poly(propylene glycol) (PPG), having a structure H[OCH(CH₃)CH₂] nOH, where n is an integer equal to or greater than 1. The linker may comprise a combination of polyalkylene oxides, such as a poly(ethylene glycol)-poly(propylene glycol)-poly(ethylene glycol) diacrylate. For example, the linker may have the following structure:

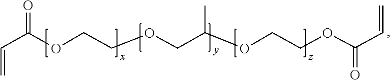

where at least one of x, y, and z is an integer greater than 0.

The polymer can be a synthetic polymer or naturally-occurring polymer. Non-limiting examples of polymers include such as a polyalkylene glycol (PAG) (e.g., polyethylene glycol (PEG) or polypropylene glycol (PPG), poly-L-lysine (PLL), poly (DL-lactic acid) (PLA), poly (DL-lactide-co-glycoside) (PLGA), polyomithine, polyarginine, deoxyribonucleic acid (DNA) or ribonucleic acid (RNA), peptides, peptoids, etc. In some embodiments, the polymer comprises (PEG). In some embodiments, the polymer comprises a nucleic acid molecule, e.g., deoxyribonucleic acid (DNA) or ribonucleic acid (RNA). In some embodiments, the polymer comprises deoxyribonucleic acid (DNA). In some embodiments, the polymer comprises ribonucleic acid (RNA).

In some embodiments, the linker comprises a cleavable linker. In some embodiments, a cleavable linker comprises a disulfide bond, a hydrazone, a PEG linker, a DNA molecule comprising a cleavage site, a peptide that is cleavable by an enzyme, an ester, or a de-click chemistry moiety. In some embodiments, a cleavable linker comprises a disulfide bond. In some embodiments, a cleavable linker comprises a hydrazone. In some embodiments, a cleavable linker comprises polyethylene glycol (PEG). In some embodiments, a cleavable linker comprises polybutylene glycol. In some embodiments, a cleavable linker comprises polypropylene glycol. In some embodiments, a cleavable linker comprises a DNA molecule comprising a cleavage site, such as a restriction site. In some embodiments, a cleavable linker comprises a peptide that is cleavable by an enzyme. In some embodiments, a cleavable linker comprises an ester. In some embodiments, a cleavable linker comprises a de-click chemistry moiety.

In some instances, a de-click chemistry moiety comprises a conjugate acceptor that can be clicked and de-clicked to starting components, such as described in Diehl et al. Nature Chemistry. 2016. 8, 968-973, which is incorporated by reference herein in its entirety. In some embodiments, a de-click chemistry moiety comprises coupling, such as clicking, of an amine and a thiol, and de-clicking, such as to result in the original amine and thiol. In some embodiments, the de-click chemistry moiety is 5-(bis(methylthio)methylene)-2,2-dimethyl-1,3-dioxane-4,6-dione. In some embodiments, de-clicking is provided by addition of a reducing agent, e.g., dithiothreitol (DTT) or tris(2-carboxyethyl)phosphine (TCEP).

In some instances, a hydrazone moiety comprises any hydrazone that may undergo hydrolysis in acidic solutions. In other instances, a hydrazone moiety comprises any hydrazone that may undergo transamination upon introduction of a nucleophilic amine. A hydrazone may be used in conjunction with a catalyst, such as an aniline-based nucleophilic catalyst as described in Nisal et al. Organic andBiomolecular Chemistry. 2018. Iss. 23, which is incorporated by reference herein in its entirety. Alternatively, a hydrazone moiety may be used without the use of a catalyst. In some embodiments, the hydrazone moiety comprises a functionalized benzylhydrazine. In some embodiments, the hydrazone moiety comprises an ortho-functionalized benzylhydrazine. In some embodiments, the hydrazone moiety may comprise 2-(hydrazineylmethyl)aniline. In some embodiments, the hydrazone moiety may comprise 2-(hydrazineylmethyl)-4,5-dimethoxyaniline. In other embodiments, the hydrazone moiety may comprise N-ethyl-2-(hydrazineylmethyl)aniline.

In some embodiments, a cleavable linker provided herein may be cleaved using any suitable mechanism, such as via application of a stimulus. In some instances, the stimulus can be, for example, a chemical stimulus, a biological stimulus, a thermal stimulus (e.g., application of heat), a photo-stimulus, a physical or mechanical stimulus, or other type of stimulus, or a combination of stimuli.

In some embodiments, cleavage of linkers may be performed via a biological stimulus, such as enzymatically (e.g., enzymatic cleavage). In some embodiments, enzymatic cleavage comprises an Edmanase. In some embodiments, enzymatic cleavage comprises an aminopeptidase, such as Pfu aminopeptidase I. In some embodiments, enzymatic cleavage comprises an exopeptidase. In some embodiments, enzymatic cleavage comprises carboxypeptidase Y. In some embodiments, enzymatic cleavage comprises acyl peptide hydrolase. In some embodiments, enzymatic cleavage comprises a restriction enzyme, nuclease, uracil DNA glycosylase transposase, Cas enzyme, or other nucleic acid-cleaving enzyme. In one such example, the linker may comprise a nucleic acid molecule (e.g., DNA) that comprises a cleavage site (e.g., restriction site, uracil, abasic site) that can be cleaved using an enzyme (e.g., restriction enzyme, uracil DNA glycosylase, nuclease, etc.). Alternatively, or in addition to, the nucleic acid molecule may comprise a partially hybridized portion or toehold region that can be de-hybridized by strand displacement (e.g., using a strand-displacing enzyme such as Phi29, or a competing nucleic acid molecule). In another example, the linker may comprise a peptide sequence, which can be cleaved using a protease (e.g., exopeptidase, aminopeptidase, proteinase K, lysC, etc.).

In some embodiments, cleavage of linkers may be performed using a chemical stimulus (e.g., chemical cleavage). In some instances, chemical cleavage comprises a change in pH, addition of a lytic agent, initiating agent, radical-generating agent, reducing agent, etc. For example, the cleavable linker may comprise a disulfide bond that is cleavable upon application of a reducing agent (e.g., DTT or TCEP). In some embodiments, chemical cleavage comprises basic conditions, such as triethylamine or potassium hydroxide. In some embodiments, chemical cleavage comprises acidic conditions, such as trifluoroacetic acid.

Other examples of cleaving stimuli include: a photo stimulus (e.g., application of UV, X-rays, gamma rays, or other wavelength of light), mechanical stimulus (e.g., sonication, high pressure, electromagnetic energy), thermal stimulus (e.g., application of heat), or chemical stimulus. In some instances, the linker comprises a labile bond that is cleavable upon application of a particular stimulus e.g., disulfide bonds (e.g., cleavable upon application of a chemical stimulus such as a reducing agent), ester linkages (e.g., cleavable with a change of pH), a vicinal-diol linkage (e.g., cleavable with sodium periodate), a Diels-Alder linkage (e.g., cleavable upon application of heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNase)).

In some embodiments, Li comprises a linkage that is non-cleavable.

In some embodiments, the linker comprises alkyl, substituted alkyl, or heteroalkyl. In some embodiments, a linker comprises alkyl, such as C₁-C₆ alkyl. In some embodiments, a linker comprises substituted alkyl, such as C₁-C₆ substituted alkyl. In some embodiments, a linker comprises heteroalkyl, such as C₁-C₆ heteroalkyl.

In some embodiments, the reactive group is linked by a covalent bond with the N-terminal amino acid of the polypeptide. In some embodiments, the capture-binding moiety is linked directly or indirectly to a substrate. In some embodiments, the reactive group is linked by a covalent bond to the N-terminal amino acid of the polypeptide that is linked to the substrate, and the capture-binding moiety is linked directly or indirectly to the substrate. In some embodiments, the capture-binding moiety is linked to a linking molecule (e.g., linking nucleic acid molecule), which can couple to a capture moiety.

### Click Chemistry Moiety

The sequencing reagents or reactive groups provide herein may comprise a click-chemistry moiety. Non-limiting examples of click-chemistry moieties are described elsewhere herein and may include alkynes, such as dibenzocyclooctyne (DBCO) groups, azides, tetrazine, TCO, and BCN.

### Methods of Using Sequencing Reagents for Sequencing Polymeric Analytes

Provided herein are methods of using any of the sequencing reagents provided herein. In some embodiments, the method comprises providing a substrate, a substrate-bound capture moiety, and a polymeric analyte. In some embodiments, the polymeric analyte is coupled to a substrate.

A substrate may comprise any useful material and assume any useful geometry (e.g., beads, spheres, particles, flat or planar surfaces, etc.), as described elsewhere herein.

In some embodiments, a substrate-bound capture moiety comprises a capture moiety bound to a substrate, as described elsewhere herein. In some embodiments, the substrate-bound capture moiety comprises a nucleic acid molecule. In some embodiments, the nucleic acid molecule is a DNA molecule, e.g., a DNA primer.

In some embodiments, a polymeric analyte comprises a biomolecule, e.g., a peptide, lipid, carbohydrate, nucleic acid molecule, or combinations or derivatives thereof, as described elsewhere herein.

In some embodiments, the method further comprises contacting the polymeric analyte with the sequencing reagent. In some embodiments, the sequencing reagent binds to a monomer of the polymeric analyte to generate a sequencing reagent-monomer complex.

In some embodiments, contacting the polymeric analyte with the sequencing reagent comprises providing reaction conditions sufficient to generate the sequencing reagent-monomer complex. Additional reagents can comprise any useful components, including buffers, catalysts, ions, metals, salts, solvents, solutes, oxidation reagents, reducing agents, etc. In some embodiments, the reaction conditions comprise the addition of Mukaiyama's reagent (2-chloro-1-methylpyridinium iodide). In other embodiments, contacting comprises contacting the polymeric analyte with the sequencing reagent with the addition of N,N-diisopropylethylamine (DIEA). In some embodiments, contacting comprises a solvent, such as dimethylformamide (DMF). In some embodiments, contacting comprises use of an aqueous solvent.

In some embodiments, the monomer comprises an amino acid residue as described elsewhere herein. In some embodiments, a monomer comprises a terminal amino acid residue.

In some embodiments, the method further comprises coupling the sequencing reagent or the sequencing reagent-monomer complex to the substrate via the substrate-bound capture moiety. In some embodiments, tethering comprises covalent attachment. In some embodiment, tethering comprises non-covalent attachment. In some embodiments, tethering comprises covalent attachment through, for example, a thiol acrylate linkage. In some embodiments, tethering comprises covalent attachment through, for example, a click-chemistry moiety, such as a click-chemistry moiety described elsewhere herein. In some embodiments, tethering comprises coupling of nucleic acid molecules via hybridization, ligation, or both, as described elsewhere herein. In one such example, the capture-binding moiety may comprise a first nucleic acid molecule that can couple to a substrate-bound capture moiety comprising a second nucleic acid molecule.

In some embodiments, the functionality of a capture-binding moiety, such as a click chemistry moiety, is analyzed by reaction of a clickable group, such as an azide, with an alkyne connected to a fluorophore. In some embodiments, the fluorophore is tetramethylrhodamine. In some instances, fluorescence is measured and increases in fluorescent intensity compared to a control is indicative of functional azide-alkyne click chemistry. In some instances, a control is a surface coated without peptide.

In some embodiments, the method further comprises cleaving the sequencing reagent-monomer complex from the polymeric analyte, thereby providing a detectable complex. In some embodiments, cleavage comprises application of a chemical stimulus (e.g., chemical cleavage). In other embodiments, cleavage comprises application of a biological stimulus (e.g., enzymatic cleavage). In some embodiments, cleavage may comprise acidic cleavage conditions, such as trifluoroacetic acid. In some embodiments, cleavage may comprise basic cleavage conditions, such as triethylamine, sodium hydroxide, or potassium hydroxide. In some embodiments, basic cleavage conditions comprise a sodium hydroxide (NaOH) solution, such as 2% NaOH. In some embodiments, enzymatic cleavage comprises an Edmanase. In some embodiments, enzymatic cleavage comprises an exopeptidase. In some embodiments, enzymatic cleavage comprises aminopeptidases, such as Pfu aminopeptidase I. In some embodiments, enzymatic cleavage comprises carboxypeptidase Y. In some embodiments, enzymatic cleavage comprises acyl peptide hydrolase. Cleavage of the sequencing reagent-monomer complex may generate a detectable complex and a remaining polymeric analyte (e.g., peptide) comprising one or more fewer monomers (e.g., amino acids) as compared to prior to the cleavage event.

In some embodiments, the method further comprises detecting the detectable complex. In some embodiments, detecting the detectable complex comprises contacting the sequencing reagent-monomer complex or the detectable complex with a binding agent. In some embodiments, the binding agent comprises an antibody, nanobody, antibody fragment, single chain variable fragment (scFv), or aptamer. In some embodiments, the binding agent comprises an antibody. In some embodiments, the binding agent comprises a nanobody. In some embodiments, the binding agent comprises a single chain variable fragment (scFv). In some embodiments, the binding agent comprises an aptamer.

In some embodiments, the binding agent comprises a detectable moiety. The detectable moiety may be a fluorescent label, a radioisotope, a metal tag, or other detectable moiety. In some embodiments, the detecting comprises imaging (e.g., via microscopy) the detectable moiety to determine the identity, or measure the binding, quantity, or other characteristic of the binding agent.

In some embodiments, the binding agent comprises a polymerizable molecule. In some embodiments, the polymerizable molecule comprises a nucleic acid molecule. The nucleic acid molecule may comprise a barcode sequence comprising identifying information of the binding agent or the binding partner of the binding agent. For example, the binding agent may comprise an antibody that is coupled to a nucleic acid barcode molecule that identifies the antibody or the antigen or target (e.g., the detectable complex, which may comprise an amino acid, or portion thereof) to which the antibody binds or recognizes.

In some embodiments, the detecting further comprises performing a nucleic acid reaction. In some instances, the binding agent may comprise an identifying nucleic acid barcode molecule coupled thereto, as described elsewhere herein, and as shown in FIG. 1A. The nucleic acid barcode molecule coupled to the binding agent may be coupled, e.g., via hybridization or ligation, to the substrate-bound capture moiety or to an additional substrate-bound capture moiety. A nucleic acid extension reaction may be performed, e.g., to copy or transfer the nucleic acid barcode molecule to the substrate-bound capture moiety or the additional substrate-bound capture moiety to generate an extended nucleic acid product. Alternatively, the nucleic acid barcode molecule may be ligated to the substrate-bound capture moiety or the additional substrate-bound capture moiety to generate a ligated nucleic acid product. In some embodiments, subsequent to generation of the extended nucleic acid product or the ligated nucleic acid product, the detectable complex may be subjected to cleavage. Cleavage may occur using chemical or enzymatic techniques, as described elsewhere herein.

In some embodiments, the detecting further comprises sequencing one or more nucleic acid molecules. For example, the ligated nucleic acid product or the extended nucleic acid product, or a derivative thereof (e.g., complement, amplicon, extended product thereof) may be sequenced, optionally following removal from the substrate. In some examples, the nucleic acid barcode molecule of the binding agent comprises the identity of the binding agent, and thus sequencing of the extended nucleic acid product or the ligated nucleic acid product yields the identity of the binding agent and the identity of the monomer (e.g., the amino acid). In some embodiments, multiple rounds of binding with binding agents may be performed prior to sequencing, as described elsewhere herein.

In some embodiments, detecting comprises mass spectrometry. In some embodiments, detecting comprises mass spectrometry performed on the analyte without the sequencing reagent, the analyte with the sequencing reagent conjugated, and the cleaved analyte after the sequencing reagent removes the terminal monomer. In some instances, detecting comprises mass spectrometry performed on the analyte without the sequencing reagent. In some instances, detecting comprises mass spectrometry performed on the analyte with the sequencing reagent conjugated, such as to the terminal monomer. In some instances, detecting comprises performing mass spectrometry on the cleaved monomer after the sequencing reagent removes the terminal monomer (e.g., the cleaved peptide). In some embodiments, detecting comprises measuring the molecular weight, such as with mass spectrometry, before cleavage and after cleavage of the terminal monomer. In some instances, the expected reduction in molecular weight by loss of one monomer, such as an amino acid, signifies successful cleavage.

In some embodiments, the method further comprises repeating any of the steps of the method, thereby sequencing the polymeric analyte. In some embodiments, the method further comprises repeating contacting the polymeric analyte with the sequencing reagent, forming a sequencing reagent-monomer complex, tethering the sequencing reagent-monomer complex to the substrate via a substrate bound capture moiety, which may be the same moiety or different moieties for each iteration, cleaving the sequencing reagent-monomer complex from the polymeric analyte, thereby providing a detectable complex, and detecting the detectable complex, thereby sequencing the polymeric analyte, as is described further below.

Systems and methods of the present disclosure may comprise contacting a monomer of a polymeric analyte with a sequencing reagent described herein to generate a sequencing reagent-monomer complex, coupling (e.g., via local tethering) the sequencing reagent or the sequencing reagent-monomer complex to a capture moiety, cleaving the sequencing reagent-monomer complex from the polymeric analyte, thereby generating a detectable complex comprising a cleaved monomer; and detecting the detectable complex.

In some instances, the detecting comprises the use of binding agents that are specific to a monomer type to recognize and bind the detectable complex. In some embodiments, detecting the detectable complex comprises contacting the sequencing reagent-monomer complex with a binding agent. In some embodiments, the monomer-specific binding agents are directly detected; for example, the binding agents may comprise a detectable label (e.g., fluorophore, mass tag, radioisotope) that can be detected upon or subsequent to a binding event; alternatively, the binding agent may be detected indirectly; for instance, the binding agent may comprise a polymerizable molecule with encoded information, which encoded information may be transferred, coupled, or copied to the capture moiety or to an additional polymerizable molecule (e.g., located adjacent to the polymeric analyte). The capture moiety or additional polymerizable molecule can be detected or read out in a subsequent process. Alternatively, or in addition to, the binding agents may be used to sort a plurality of detectable complexes, e.g., by their constituent monomer types, into separate partitions or compartments for downstream labeling, e.g., with identifying barcode molecules.

In some instances, the detecting comprises direct detection of the detectable complex. For instance, after generating the sequencing reagent-monomer complex and cleaving the sequencing reagent-monomer complex to thereby generate the detectable complex, the detectable complex may be detected by using a nanopore sequencer (e.g., a commercially available nanopore sequencer, such as those provided by Oxford Nanopore Technologies).

Any of the operations may be iterated or repeated any number of times to obtain information on all or a subset of the monomers of the polymeric analyte and optionally, the sequence of the monomers relative to the polymeric analyte. In some instances, the information may be read out from the polymerizable molecules described herein using, for example, conventional next-generation sequencing approaches. Beneficially, by cleaving the monomer from the polymeric analyte, the monomers are removed from the adjacent monomers, and binding agents can specifically bind to individual monomers without the influence of the adjacent surrounding monomers. As such, the methods disclosed herein enable more accurate molecular identification and polymeric sequencing, which has applications in diagnosing disease, monitoring protein dynamics or protein interactions, single-cell proteomics, developing or characterizing therapeutics, and more.

One or more methods of the present disclosure may employ the use of a sequencing reagent described herein; the sequencing reagent may function as a linker that is capable of coupling to (i) a monomer of the polymeric analyte and (ii) a capture moiety, which may be used to locally tether the monomer, once cleaved, adjacent to the polymeric analyte. The methods and systems disclosed herein may additionally, in some embodiments, comprise a substrate for local tethering; the substrate may be coupled, for example, to the polymeric analyte, the capture moiety, and an additional polymerizable molecule. In some instances, the additional polymerizable molecule is encoded with information from a polymerizable molecule of a binding agent. In other instances, the binding agent comprises a detectable label that can directly indicate a binding event between the binding agent and the monomer.

Methods of the present disclosure for processing a polymeric analyte comprising a plurality of monomers may comprise cleaving of a monomer of the polymeric analyte and coupling the monomer to a capture moiety (e.g., coupled to a substrate, or suspended in solution) for subsequent processing or analysis. In one example, a method of the present disclosure may comprise: providing (i) a polymeric analyte comprising a plurality of monomers and (ii) a capture moiety; coupling a monomer of the plurality of monomers to the capture moiety to generate a monomer-capture moiety complex; cleaving the monomer; contacting the cleaved monomer-capture moiety complex with the binding agent; and coupling a first polymerizable molecule to a second polymerizable molecule or to the capture moiety. In some instances, the first polymerizable molecule is coupled to the binding agent and comprises information about the binding agent, such as the identity of the binding agent or its cognate molecule, which information may be transferred to the second polymerizable molecule or to the capture moiety. In some instances, the capture moiety may be a copy or identical molecule as the second polymerizable molecule. Alternatively, or in addition to, the binding agent may be used to sort a mixture of cleaved monomers by identity or type, and subsequent to sorting, an identifying label or barcode that identifies the monomer type may be coupled to the capture moiety. Example methods and systems of such processing approaches and systems are described in U.S. Pat. No. 11,499,979, International Pat. App. No. PCT/US2023/017954, and U.S. Provisional Pat. App. No. 63/507,558, filed Jun. 12, 2023, each of which is incorporated by reference herein in its entirety.

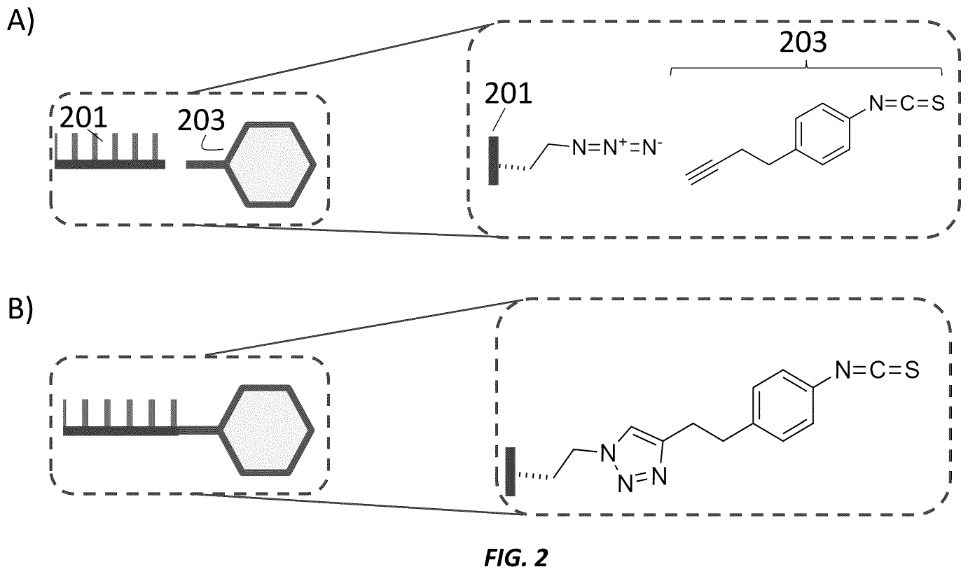

FIG. 1A shows an example workflow of analyzing a polymeric analyte. In workflow 100, a polymeric analyte 103 is provided, sequentially disassembled into individual monomers via contacting with a sequencing reagent and coupling to capture moieties, monomer cleavage, and indirect detection using binding agents comprising polymerizable molecules that identify or encode for the binding agents. The polymerizable molecule of a binding agent is coupled to an additional polymerizable molecule, and the monomer is cleaved from the capture moiety or blocked to prevent downstream recognition from additional binding agents, thereby allowing for iteration of the process to analyze all or a subset of the individual monomers comprised by the polymeric analyte. In FIG. 1A Panel A, a substrate 101 is coupled to a polymeric analyte 103 (e.g., a peptide to be sequenced), a capture moiety 105 (e.g., a nucleic acid primer), and an additional polymerizable molecule 107 (e.g., an additional nucleic acid primer). In some instances, the capture moiety 105 and the additional polymerizable molecule 107 are identical molecules (e.g., comprise the same sequence). The polymeric analyte may be contacted with a sequencing reagent 109 (e.g., a sequencing reagent of Formulas I, IV, IV′, IV″, IV-A, IV-B, or V-C), which comprises a terminal monomer-coupling group (e.g., an amino acid-reactive group comprising a guanidinylating agent) and a capture-binding moiety comprising a click chemistry moiety (e.g., azide). In some instances, the sequencing reagent 109 couples to a terminal monomer (e.g., terminal amino acid such as the N-terminal amino acid (NTAA)). The coupling results in formation of a sequencing reagent-monomer complex. In FIG. 1A Panel B, a linking nucleic acid molecule 111 comprising a click chemistry moiety (e.g., alkyne) is reacted with the sequencing reagent 109 of the sequencing reagent-monomer complex and covalently linked. In some instances, the linking nucleic acid molecule 111 and the sequencing reagent 109 are provided pre-coupled (see, e.g., FIG. 2). In such instances, the linking nucleic acid molecule 111 may constitute the capture-binding moiety of the sequencing reagent. In FIG. 1A Panel C, the linking nucleic acid molecule 111 is coupled to the capture moiety 105, thereby generating a monomer-capture moiety complex comprising the sequencing reagent-monomer complex coupled to the capture moiety 105. The coupling may be mediated by hybridization of the linking nucleic acid molecule 111 to the capture moiety 105 (hybridization not shown), or by using a splint oligonucleotide 113 comprising sequences complementary to a sequence of the linking nucleic acid molecule 111 and the capture moiety 105. In some instances (not shown), the linking nucleic acid molecule 111 comprises a self-splinting sequence, such that the linking nucleic acid molecule 111 may couple to the capture moiety 105 in the absence of a separate splint molecule. A ligase may be used to covalently link the linking nucleic acid molecule 111 to the capture moiety 105. Alternatively, the linking nucleic acid molecule 111 may comprise a first reactive moiety (e.g., click chemistry moiety not shown) that can react with a second reactive moiety (not shown) of the capture moiety 105. In FIG. 1A Panel D, the monomer-capture moiety complex is subjected to conditions sufficient to cleave the terminal monomer (e.g., amino acid) from the polymeric analyte 103 (e.g., peptide), thereby generating a detectable complex comprising the cleaved monomer. The conditions may include performing an Edman-like degradation reaction. The cleavage of the monomer from the polymeric analyte results in a cleaved monomer-capture moiety complex comprising the cleaved monomer, sequencing reagent 109, linking nucleic acid molecule 111, and capture moiety 105. In FIG. 1A Panel E, a binding agent 115 (e.g., antibody) comprising another polymerizable molecule 117 (e.g., nucleic acid molecule) is provided and contacted with the detectable complex. The binding agent 115 may be specific to the monomer (e.g., to an amino acid type) or to the sequencing reagent-monomer complex (e.g., sequencing reagent-amino acid complex). In some instances, the binding agent 115 recognizes and binds to the sequencing reagent-monomer complex and not the monomer when still attached to the polymeric analyte. The polymerizable molecule 117 of the binding agent may comprise information on the identity of the binding agent or the specific monomer (e.g., single amino acid) to which the binding agent binds. The polymerizable molecule 117 of the binding agent may comprise additional sequences, such as a barcode sequence, UMI, restriction site, transposition site, a sequence to represent a cycle or iteration number, or other functional sequence. The polymerizable molecule 117 of the binding agent may couple to the additional polymerizable molecule 107 that is coupled to the substrate 101. In some instances, an extension reaction may be performed (e.g., using a polymerase), to copy the sequence of the polymerizable molecule 117 of the binding agent to the additional polymerizable molecule 107 that is coupled to the substrate 101. Alternatively, the polymerizable molecule 117 of the binding agent may be ligated to the additional polymerizable molecule 107, either chemically (e.g., via complementary click chemistry) or enzymatically (e.g., using a ligating enzyme, ribozyme or DNAzyme). Optionally, the polymerizable molecule 117 may be cleaved from the binding agent 115 (not shown). In FIG. 1A Panel F, the monomer of the detectable complex may be decoupled (e.g., removed or cleaved) from the capture moiety 105. For example, the monomer, sequencing reagent 109, and all or a portion of the linking nucleic acid molecule 111 may be cleaved (depicted as a star). The cleavage may be performed chemically, mechanically, or enzymatically. In an example of enzymatic cleavage, the linking nucleic acid molecule 111 may comprise a restriction site or other cleavage site (e.g., a uracil), and cleavage occurs by introduction of a restriction enzyme or cleaving enzyme (e.g., uracil DNA glycosylase) to cleave the restriction/cleavage site. Alternatively, the cleaved monomer-capture moiety complex may be blocked with a blocking agent (not shown). The workflow 100 may then be iterated or repeated to sequence all or a portion of the polymeric analyte 103.

FIG. 1B schematically shows another example of processing and characterizing a polymeric analyte 103, e.g., a peptide. A polymeric analyte 103 may be tagged (or provided pre-tagged) with a capture moiety 105, e.g., a polymerizable molecule such as a nucleic acid molecule. The capture moiety 105 may, for example, be attached at a terminus of a peptide (e.g., as shown at the C-terminus) or at an internal residue. The capture moiety 105 may, in some instances, comprise a barcode sequence, e.g., a barcode that identifies the polymeric analyte, the sample origin, a partition or compartment, etc. In some instances, the capture moiety 105 may not be attached to the peptide but may be associated to the peptide (e.g., via an indirect interaction). In other examples (not shown), the peptide is coupled (directly or indirectly) to a non-nucleic acid molecule (e.g., a polymerizable molecule such as an additional peptide, or other detectable label such as a mass tag, fluorophore, radioisotope, etc.). A sequencing reagent 109, e.g., a sequencing reagent of Formulas I, IV, IV′, IV″, IV-A, IV-B, or V—C comprising a linking nucleic acid molecule 111 is provided. The linking nucleic acid molecule 111 may comprise any useful sequences, such as a primer sequence, a barcode sequence, a UMI, restriction site, etc. and may comprise a capture-binding moiety. In some instances, the linking nucleic acid molecule 111 comprises a cleavable moiety, e.g., a restriction site, an abasic site, a uracil, a transposition site, etc. In process 110, the sequencing reagent 109 couples to a monomer (e.g., terminal amino acid) of the polymeric analyte to generate a sequencing reagent-monomer complex. Prior to, during, or subsequent to the coupling of the sequencing reagent to the monomer, the linking nucleic acid molecule 111 may couple to the capture moiety 105 via the capture-binding moiety, e.g., via hybridization (not shown), ligation (shown as process 112), or splinted ligation (not shown), thereby generating a monomer-capture moiety complex. In process 113, the monomer is cleaved (e.g., chemically or enzymatically) from the rest of the polymeric analyte 103, thereby providing a detectable complex comprising the cleaved monomer-capture moiety complex. The cleaved monomer-capture moiety complex comprises the cleaved monomer coupled to the sequencing reagent 109, linking nucleic acid molecule 111, and capture moiety 105. In some instances, the cleaved monomer-capture moiety complex remains coupled to the polymeric analyte 103 via the linking nucleic acid molecule 111 and the capture moiety 105. The detectable complex comprising the cleaved monomer-capture moiety complex can be detected directly or further processed for downstream analysis.

The downstream processing and analysis may comprise sorting, detection, or both. In some instances, a plurality of binding agents 115 is provided; the binding agents 115 may recognize and bind different monomer types (e.g., different amino acid types). In some instances, the plurality of binding agents 115 recognize and bind to the sequencing reagent-monomer complexes (e.g., recognize and bind to the different monomer types comprised by the sequencing reagent-monomer complexes). In some instances, the plurality of binding agents 115 recognize and bind to the sequencing reagent-monomer complexes and not the monomer when still attached to the polymeric analyte. The binding agents 115 may be contacted with a plurality of cleaved monomer-capture moiety complexes comprising different monomer types (e.g., different amino acids) and bind to their respective targets. In some instances (not shown), the binding agents 115 comprise polymerizable molecules, e.g., nucleic acid molecules, that identify the binding agent or its cognate molecule; the polymerizable molecules may be coupled or transferred to the capture moiety 105 (not shown), e.g., via nucleic acid extension, ligation, transposition, etc. Alternatively or in addition to, in process 119, the binding agents 115 may be separated or sorted, e.g., using complementary nucleic acid sequences to the nucleic acid molecules of the binding agents, into separate compartments (not shown). Alternatively, or in addition to, the binding agent may comprise a sorting tag, e.g., a reporter molecule, mass tag, fluorophore or fluorescent protein, which can enable sorting of the different binding agent types. In one such example, a first binding agent against a first monomer type (e.g., an amino acid residue) may comprise a GFP tag and a second binding agent against a second monomer type may comprise an RFP tag that is sortable by fluorescence (e.g., using FACS) or affinity sorting (e.g., using beads with anti-GFP and anti-RFP antibodies).

In some instances, subsequent to process 119, the linker 109 or the linking nucleic acid molecule 111 or portion thereof may be removed from the monomer-linker complex or cleaved monomer-linker complex, e.g., via restriction digest or cleavage of a uracil (e.g. using UDG or USER enzymes) of the linking nucleic acid molecule 111.

In some instances, barcoding of the cleaved monomer-capture moiety complexes may be performed. For example, as described above, the binding agents 115 may comprise polymerizable barcode molecules, e.g., nucleic acid barcode molecules, that identify the binding agent or its cognate molecule; the polymerizable barcode molecules may be coupled or transferred to the capture moiety 105 (not shown), thereby barcoding the capture moiety. Alternatively, or in addition to, subsequent to sorting in process 119, the sorted cleaved monomer-capture moiety complexes may be barcoded. For instance, subsequent to sorting, the cleaved monomer-capture moiety complexes may be compartmentalized by their corresponding monomer type (e.g., amino acid type). As each compartment comprises a known monomer type (e.g., amino acid type) according to the binding profile (e.g., specificity to a particular monomer type) of the binding agent, the cleaved monomer-capture moiety complex within a compartment may be labeled with an identifying polymerizable molecule 117 (e.g., nucleic acid barcode molecule) that comprises the identity of the particular monomer type of the cleaved monomer-capture moiety complex, thereby generating a barcoded capture moiety.

Subsequent to barcoding, the contents of the separate compartments may then be pooled, and the process repeated to iteratively cleave and attach an identifying barcode for each monomer in the polymeric analyte. The subsequent barcode molecules may attach to the barcoded capture moiety to generate a multi-barcoded additional polymerizable molecule (e.g., a concatenated or stacked barcoded nucleic acid molecule). Alternatively, or in addition to, the barcodes may be added onto separate polymerizable molecules, e.g., amplicons (not shown) of the capture moiety 105. In some instances, the polymerizable molecules 117 or the linking nucleic acid molecule 111 may comprise temporal information (e.g., a round or cycle number). After any useful number of rounds or iterations, the barcoded additional polymerizable molecule (or multi-barcoded additional polymerizable molecule) may be removed and sequenced, e.g., using NGS approaches, thereby outputting the identity of each monomer type that has been processed and the order or position in which they occur, based on the temporal information, in the polymeric analyte.

FIG. 1C schematically shows another example workflow of sequencing a polymeric analyte. A plurality of polymeric analytes (e.g., peptides) may be coupled to a substrate 101, along with a plurality of capture moieties. For illustration purposes, further sequencing workflow operations are shown for a single polymeric analyte 103; however, it will be appreciated that the workflow operations of FIGS. 1A-1F may be performed on a plurality of polymeric analytes in parallel. In process 110, a sequencing reagent 109 (e.g., a sequencing reagent of Formulas I, IV, IV′, IV″, IV-A, IV-B, or V-C) is provided and coupled to a monomer (e.g., terminal amino acid), thereby generating a sequencing reagent-monomer complex. The sequencing reagent 109 is capable of coupling to (i) a monomer of the polymeric unit (e.g., a terminal amino acid, such as the N-terminal amino acid) and (ii) a capture moiety 105, which may be used to locally tether the monomer adjacent to the polymeric analyte. In an example, the sequencing reagent 109 may comprise an amino acid-reactive group such as a guanidinylating agent, which enables the sequencing reagent to couple to an N-terminal amino acid. The sequencing reagent may additionally comprise a capture-binding moiety that can couple to the capture moiety and may serve as a substrate-tethering moiety. For example, the capture-binding moiety may comprise a click chemistry moiety that can couple to a click chemistry capture moiety, e.g., through an azide-alkyne or azide-cycloalkyne reaction. In other examples, the capture-binding moiety may comprise a nucleic acid molecule that can couple, e.g., via hybridization, ligation, or both, to a nucleic acid capture moiety (e.g., as shown in FIG. 1A). In process 112, the capture-binding moiety of the linker may couple to the capture moiety. In process 113, the monomer may be cleaved from the polymeric analyte, thereby generating a detectable complex. In some embodiments, cleavage of the monomer may be mediated by a stimulus, e.g., chemical reaction or pH change (such as addition of acid). Subsequent to cleavage, a binding agent 115 is provided. The binding agent, e.g., an antibody or antibody fragment, may be specific to a particular monomer of a plurality of monomers, e.g., specific to a particular amino acid type or derivative thereof. In some instances, the binding agent 115 recognizes and binds to the sequencing reagent-monomer complex and not the monomer when still attached to the polymeric analyte. The binding agent may comprise a detectable label (e.g., fluorophore, radioisotope, mass tag, etc.). The detectable label may be detected (e.g., using microscopy or imaging). To determine the identity of the cleaved terminal monomer (e.g., amino acid). Subsequently, the detectable complex or portion thereof, e.g., the sequencing reagent or the sequencing reagent-monomer complex, may be removed or cleaved, and the process may be repeated or reiterated to sequence the remaining monomers of the polymeric analyte.

FIG. 1D shows another workflow of sequencing a polymeric analyte. A polymeric analyte 103 and a capture moiety 105 are provided, which optionally are coupled to a substrate 101. The capture moiety 105 may comprise a first nucleic acid molecule (e.g., DNA). In process 106, a sequencing reagent 109 comprising a polymerizable molecule, e.g., a linking nucleic acid molecule 111 are provided. In some instances, the sequencing reagent 109 is pre-tethered to the polymerizable molecule (depicted as a linking nucleic acid molecule 111); alternatively, the sequencing reagent 109 and the polymerizable molecule may be provided separately. In process 106, the sequencing reagent 109 may couple to a monomer, e.g., an amino acid (e.g., NTAA) of the polymeric analyte 103 (e.g., a peptide) to generate a sequencing reagent-monomer complex. In process 112, the sequencing reagent-monomer complex may couple to the capture moiety 105, thereby generating a monomer-capture moiety complex. Coupling of the sequencing reagent-monomer complex to the capture moiety 105 may be mediated by the polymerizable molecule, e.g., the linking nucleic acid molecule 111. Optionally, the sequencing reagent-monomer complex and the capture moiety 105 may be covalently linked together (e.g., the linking nucleic acid molecule 111 may be covalently linked to the capture moiety 105), using chemical (e.g., click chemistry) or enzymatic (e.g., a ligase) approaches. Alternatively, or in addition to, the polymerizable molecule may comprise a first sequence that is complementary and may hybridize to a second sequence of the capture moiety 105 (not shown), or the polymerizable molecule may be linked to the capture moiety 105 via a splint or bridge molecule, which may comprise sequences that are complementary to the first sequence of the polymerizable molecule and the second sequence of the capture moiety 105 (not shown). In process 113, the monomer is cleaved from the polymeric analyte 103, thereby providing a detectable complex which comprises a cleaved monomer-capture moiety complex comprising the cleaved monomer coupled to the sequencing reagent 109, polymerizable molecule (shown as a linking nucleic acid molecule 111), and capture moiety 105, or a portion thereof In process 114, a binding agent 115 (e.g., an antibody, binding protein, etc.) may be contacted with the detectable complex. The binding agent may be configured to recognize all or a portion of the cleaved monomer-capture moiety complex. For example, the binding agent may recognize the monomer, the sequencing reagent-monomer complex, or the entirety of the monomer-capture moiety complex. In one example, the sequencing reagent may comprise a guanidinylating agent, and the binding agent may recognize the reacted guanidinylating agent with the amino acid, or a derivative thereof. In some instances, the binding agent 115 may comprise a detectable moiety (not shown) or may be contacted with an additional binding agent (e.g., a secondary antibody) which may optionally comprise a detectable moiety (not shown).

Any of the processes, e.g., 106, 112, 113, or 114 may be iterated and repeated any number of times (“rounds”) using additional sequencing reagents 109 and polymerizable molecules (optionally comprising cycle/round information), and tethering the additional polymerizable molecules together (e.g., tethering an additional polymerizable molecule to the polymerizable molecule of the monomer-capture moiety complex). Multiple rounds may be performed until all or a subset of the monomers in the polymeric analyte 103 are cleaved and tethered together. In some instances, processes 106, 112, and 113 may be iterated to generate a stacked polymerizable molecule 123 comprising a set of cleaved monomers, e.g., a concatenated set of sequencing reagent-monomer-polymerizable molecule complexes. The stacked polymerizable molecule may then be contacted with a library of binding agents, which can bind to their respective monomer targets (e.g., an amino acid type).

Further downstream analysis may be performed, e.g., using a nanopore or nanogap system. In one such example, the stacked polymerizable molecule 123, which optionally may be coupled to binding agents, may be prepared and translocated through a nanopore using a nanopore sequencing system (e.g., commercially available system from Oxford Nanopore Technologies), which may output the identity of the polymerizable molecules (e.g., a nucleic acid sequence), the monomer type (e.g., amino acid identity), and, if prevalent, the individual binding agents. In nanopore sequencers, individual analytes enter the nanopore under an applied electric potential, thereby altering the flow of ions through the nanopore in a time-dependent manner. Measurement of the modulation of the ionic current as the individual analytes translocate through the nanopore can be performed, and the measured signal can be computationally decoded, e.g., to yield a DNA sequence of a DNA analyte. Similarly, a nanopore sequencer may be used to characterize or analyze the detectable complexes described herein, e.g., a sequencing reagent-monomer-polymerizable complex or a concatenated set of sequencing reagent-monomer-polymerizable molecule complexes. Additional example methods and systems for nanopore sequencing of intramolecularly expanded peptides can be found in International Patent App. No. PCT/US2023/071456, which is incorporated by reference herein.

Alternatively, in some instances, no binding agents may be required in order to sequence the polymerizable molecule. FIG. 1E schematically illustrates an example workflow of sequencing a polymeric analyte. In such an example workflow, the same operations of the workflow shown in FIG. 1D are performed, omitting process 114. The workflow may be iterated numerous times to generate a stacked polymerizable molecule 123, which may be further processed, e.g., analyzed using a nanopore or nanogap sequencing system, to output the identity of the polymerizable molecules and the individual monomers.

Similarly, FIG. iF schematically illustrates another example workflow of sequencing a polymeric analyte in the absence of binding agents and that can be prepared in the absence of a substrate (e.g., in solution), or on a substrate (FIG. iF inset). In such an example, a polymeric analyte 103 and a capture moiety 105 are provided. The capture moiety 105 may comprise a first nucleic acid molecule (e.g., DNA molecule) and may comprise identifying information of the polymeric analyte 103, e.g., an identifying barcode sequence. The capture moiety 105 may be coupled to the polymeric analyte 103. The capture moiety 105 may additionally comprise a releasable or cleavable moiety (e.g., a uracil, abasic site, restriction site, etc.). In process 106, a sequencing reagent 109 and polymerizable molecule, such as a linking nucleic acid molecule 111, are provided. In some instances, the sequencing reagent 109 is pre-tethered to the polymerizable molecule (linking nucleic acid molecule 111); alternatively, the sequencing reagent 109 and the polymerizable molecule (linking nucleic acid molecule 111) may be provided separately. The polymerizable molecule may comprise identifying temporal information, e.g., the cycle or round in which it is provided. In process 106, the sequencing reagent 109 may couple to a monomer, e.g., an amino acid (e.g., NTAA) of the polymeric analyte 103 to generate a sequencing reagent-monomer complex. In process 112, the sequencing reagent-monomer complex may couple to the capture moiety 105. Coupling of the sequencing reagent-monomer complex to the capture moiety 105 may be mediated by the polymerizable molecule (linking nucleic acid molecule 111) and optionally an additional polymerizable molecule 116 (e.g., a splint molecule, a bridge molecule, etc.). Optionally, the sequencing reagent-monomer complex and the capture moiety may be covalently linked together (e.g., via ligation, click chemistry, etc.). Alternatively, or in addition to, the polymerizable molecule may comprise a first sequence that is complementary and may hybridize to a second sequence of the capture moiety 105 (not shown), or the polymerizable molecule may be linked to the capture moiety 105 via a splint or bridge molecule, which may comprise sequences that are complementary to the first sequence of the polymerizable molecule and the second sequence of the capture moiety 105 (not shown). In process 113, the monomer may be cleaved from the polymeric analyte 103 to generate monomer-capture moiety complex, which comprises the cleaved monomer, the sequencing reagent 109, the polymerizable molecule (e.g., linking nucleic acid molecule 111), and the capture moiety 105. Processes 106, 112, and 113 may be iterated and repeated any number of times (“rounds”) using additional sequencing reagents 109 and polymerizable molecules, and tethering the additional polymerizable molecules together (e.g., tethering an additional polymerizable molecule to the monomer-capture moiety complex). Multiple rounds may continue until all or a subset of the monomers of the polymeric analyte 103 are tethered together. For example, the process may be iterated to generate a stacked polymerizable molecule 123 comprising a set of cleaved monomers, e.g., a concatenated set of monomer-sequencing reagent-polymerizable molecule complexes. After any useful number of rounds, the stacked polymerizable molecule 123 may be cleaved from the or at the capture moiety 105, e.g., using the cleavable moiety. The cleaved product may then be sequenced using a nanopore or nanogap sequencer, which can output the identity of each cleaved monomer, the sequencing reagent-monomer complex, the polymerizable molecule (e.g., a DNA sequence of a DNA molecule), or a combination thereof.

In some instances, the polymerizable molecule, e.g., linking nucleic acid molecule 111 comprises temporal information on the cycle in which it is provided; as such, the temporal information may be used for conducting quality control. For example, if a missing cycle number is missing, then it can be inferred that an amino acid is missing or was not present in the peptide, that cleavage of the amino acid did not occur, or other error. Alternatively, or in addition to, temporal barcodes may be provided separately and attached to the polymerizable molecule at any useful or convenient step.

Iteration: In some instances, one or more of the operations described herein may be iterated or repeated. Iteration of the operations may allow for sequential processing, analysis, or identification of the individual monomers of the polymeric analyte, which can allow for reconstruction of the entire polymeric analyte. For example, referring to FIG. 1A, the operations of the workflow 100 may be conducted to encode the identity (e.g., via the polymerizable molecule 117) of a terminal amino acid (e.g., NTAA) onto the additional polymerizable molecule 107. The operations of workflow 100 may then be repeated to encode the identities of the n-1 position monomer, n-2 position monomer, etc., until the entire or portion of the polymeric analyte, e.g., peptide, is processed. The encoding may occur on the same (additional) polymerizable molecule 107, e.g., to generate a stacked polymerizable molecule comprising multiple polymerizable molecules from multiple binding agents, or the encoding may occur on additional polymerizable molecules (not shown) present on the substrate. In the former situation, in some instances, the polymerizable molecule of the second (or third, fourth, fifth, . . . nth) cycle may be configured to only couple to the first (or second, third, fourth, . . . n-lth) polymerizable molecule. For example, the first cycle binding agent polymerizable molecule may comprise a unique binding sequence that is absent on the additional polymerizable (or capture) molecules of the substrate, and to which the second cycle binding agent polymerizable molecule can bind. Accordingly, the second cycle binding agent polymerizable molecule can only bind to the first cycle binding agent polymerizable molecule and not to any of the additional polymerizable (or capture) molecules of the substrate. In the event that no binding occurs (a “null” event), a bridging polymerizable molecule may be provided that encodes for a null binding event but comprises the unique binding sequence, such that subsequent rounds may continue, even if a binding agent does not bind the cleaved monomer.

Similarly, any of the operations depicted in FIG. 1B-1F may be iterated to sequentially analyze all or a subset of the monomers of the polymeric analyte. Referring to FIG. 1B, the operations of the workflow may be conducted to encode the identity (e.g., via the polymerizable molecule 117) of a terminal monomer (e.g., NTAA) onto an additional polymerizable molecule (not shown) or onto the capture moiety. For a peptide analyte, the operations may be repeated to encode the identities of the n-1 terminal amino acid, the n-2 terminal amino acid, the n-3 terminal amino acid, etc. until the entire or portion of the peptide is processed. The encoding may occur on the same capture moiety 105, e.g., to generate a stacked polymerizable molecule comprising multiple barcode polymerizable molecules 117, or the encoding may occur on additional polymerizable molecules (not shown). In the former situation, in some instances, the polymerizable molecule of the second (or third, fourth, fifth, . . . nth) cycle may be configured to only couple to the first (or second, third, fourth, . . . n-lth) polymerizable molecule, as described above.

In instances where one or multiple additional polymerizable molecules are used, the polymerizable molecules 117 (e.g., coupled to the binding agents or provided separately after sorting) may additionally encode for the cycle or iteration number, such that the order of the individual monomers may be determined. For example, for a given peptide, the terminal amino acid may be coupled to a capture moiety and cleaved, then contacted with a binding agent comprising a barcode sequence that identifies (i) the identity of the amino acid (e.g., any one of twenty proteinogenic amino acids) and (ii) the cycle number (e.g., cycle 1) (not shown). The information encoded by the barcode sequence may be coupled to an adjacent (additional) polymerizable molecule (not shown) or to the capture moiety 105 or barcoded capture moiety. Following cleavage of the monomer from the capture moiety (e.g., as shown in process 121 of FIG. 1B), the workflow may be repeated for the n-1 terminal amino acid, which may again be coupled to a capture moiety, cleaved, and contacted with a binding agent, which may comprise an additional barcode sequence that identifies (i) the identity of the amino acid (e.g., any one of twenty proteinogenic amino acids) and (ii) the cycle number (e.g., cycle 2). The information encoded by the additional barcode sequence may be transferred to the same capture moiety 105 or barcoded capture moiety, or to an additional polymerizable molecule (not shown). In the former situation, the polymerizable molecule may then comprise information on the (i) the identity of the terminal amino acid, (ii) the cycle number of the terminal amino acid (cycle 1), (iii) the identity of the n-1 terminal amino acid, and (iv) the cycle number of the n-1 terminal amino acid (cycle 2), and so forth.

Alternatively, or in addition to, the binding agent may comprise a sorting tag, and a barcode sequence may be provided subsequent to sorting. For example, the binding agents may be used to sort different cleaved monomer-capture moiety complexes (as shown in process 119), and in process 123, a polymerizable molecule 117 comprising the identity of the amino acid type may be provided for each sorted, cleaved monomer-capture moiety complex. The polymerizable molecule 117 may also comprise temporal information, e.g., the round or cycle in which it is provided.

In some instances, temporal information may be provided separately. For example, prior to, during, or subsequent to coupling of a polymerizable molecule 117 comprising barcode information to either an additional polymerizable molecule 107 (FIG. 1A) or the capture moiety (FIG. 1B), a temporal barcode may be provided that can couple to the polymerizable molecule 117 (FIGS. 1A-1B), the additional polymerizable molecule 107 (FIG. 1A), the capture moiety 105 (FIG. 1B, 1D-1F), or a combination thereof. The temporal barcode may comprise any useful agent, including a nucleic acid molecule, a peptide, a lipid, a carbohydrate, an enzyme (e.g., a chromogenic or fluorogenic enzyme) or a ribozyme or DNAzyme, a fluorophore, a dye, an intercalating agent, a dideoxynucleotide, a fluorescent nucleic acid molecule or nucleotide, a radioisotope, a mass tag, or other detectable label that can indicate the time or cycle (or iteration) number in which it is provided. In some instances, the temporal barcode comprises a cycle-specific nucleic acid barcode molecule, which can couple to the polymerizable molecule 117 (comprising the identity of the monomer) or to a stacked polymerizable molecule comprising polymerizable molecules from multiple rounds or iterations. The temporal barcode may comprise any additional useful functional sequences, e.g., primer sites, sequencing sites, restriction sites, abasic or cleavable sites, etc. In some instances, the temporal barcode may comprise an amplification site that allows for bridge amplification of the temporal barcode and optionally, the coupled polymerizable molecules, to other capture or polymerizable molecules.

Any number of operations may be iterated any useful number of times. For instance, subsequent to contacting the detectable complex with a binding agent or plurality of binding agents, the detectable complex may be washed any number of times to remove any unbound binding agent. Any of the operations of the methods described herein may be iterated. For example, the method may comprise contacting another monomer of the polymeric analyte (e.g., n-1 NTAA) with the same or different sequencing reagent described herein to generate an additional sequencing reagent-monomer complex, coupling (e.g., via local tethering) the sequencing reagent or the additional sequencing reagent-monomer complex to the same or different capture moiety (or to a polymerizable molecule such as 117 of FIG. 1B), cleaving the additional sequencing reagent-monomer complex from the polymeric analyte, thereby generating an additional detectable complex comprising another cleaved monomer; and detecting the additional detectable complex. The operations described herein may be repeated any useful number of times, e.g., until all or a portion of the polymeric analyte is processed or analyzed.

Polymeric Analytes: The polymeric analyte may be a biomolecule, macromolecule, or synthetic molecule. The polymeric analyte may be a biomolecule or other biological molecule that comprises one or more monomers. Non-limiting examples of polymeric biomolecules include nucleic acid molecules (e.g., DNA molecule, RNA molecule, DNA:RNA hybrids, aptamers), peptides and proteins, polysaccharides, lipid polymers (e.g., diglycerides, triglycerides and other fatty acids). The polymeric analyte may be a synthetic molecule, e.g., a peptoid or synthetic polymer, or a peptidomimetic (e.g., a peptoid, a beta-peptide, a D-peptide peptidomimetic). Non-limiting examples of synthetic polymers include acrylics, nylons, silicones, viscose, rayon, polyesters, polycarboxylic acids, polyvinyl acetate, polyacrylamide, polyacrylate, polyethylene glycol, polyurethane, polylactic acid, silica, polystyrene, polyacrylonitrile, polybutadiene, polycarbonate, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride), or a combination thereof. The polymeric analytes may comprise a single polymer type (e.g., a homopolymer) or more than one polymer type (e.g., a copolymer) and may comprise random or arranged monomers. The polymeric analytes may be a block polymer, alternating copolymer, periodic copolymer, statistical copolymer, stereoblock copolymer, gradient copolymer, branched copolymer, graft copolymer, etc.

The polymeric analytes may be any size or comprise a range of sizes. The polymeric analyte may be about 1 nanometer (nm), about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1 micrometer (m), about 10 m, about 100 m, about 1 millimeter mm in size or greater. A plurality of polymeric analytes may comprise polymeric analytes of similar size or within a range of sizes, e.g., between about 10 nm to about 100 nm, between about 50 nm to about 1 m. Similarly, the polymeric analytes may have any molecular weight or range of molecular weights. The polymeric analytes may be about 10 daltons (Da), 100 Da, 500 Da, 1 kilodalton (kDa), 10 kDa, 100 kDa, 1,000 kDa, 10,000 kDa, 100,000 kDa, or greater. The polymeric analytes may comprise polymeric analytes of similar molecular weight or within a range of molecular weights.

The monomers of the polymeric analytes may comprise any size or range of sizes that is less than that of the entire polymeric analyte. A monomer may be about 0.1 nanometer (nm), about 0.5 nm, 1 about 1 nm, about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1 micrometer (m), about 10 m, about 100 m, about 1 millimeter mm in size or greater. The monomers may have any molecular weight or range of molecular weights. The monomers may be about 1 dalton (Da), 10 Da, 100 Da, 500 Da, 1 kilodalton (kDa), 10 kDa, 100 kDa, 1,000 kDa, 10,000 kDa, 100,000 kDa, or greater. The monomers or polymeric analytes may range in size of molecular weight; for example, a polymeric analyte may comprise a peptide comprising amino acid monomers, which may vary in molecular weight from 75 Da (glycine) to 204 Da (tryptophan).

The polymeric analytes may comprise any number of monomers. The polymeric analytes may comprise about 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 50,000, 100,000 or more monomers. The polymeric analytes may comprise at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 500, at least about 1,000, at least about 5,000, at least about 10,000, at least about 50,000 at least about 100,000 or greater monomers. Alternatively, the polymeric analytes may comprise at most about 100,000, at most about 50,000, at most about 10,000, at most about 5,000, at most about 1,000, at most about 500, at most about 100, at most about 50, at most about 10, at most about 5, or fewer monomers. The polymeric analytes may comprise a range of monomers; for example, a polymeric analyte may comprise about 5 monomers whereas another polymeric analyte may comprise about 500 monomers.

In some instances, the polymeric analyte comprises a peptide comprising amino acid monomeric units. The peptide may be naturally occurring or synthetic. The peptide may comprise any number of amino acids. The amino acids may be one of 20 proteinogenic amino acids and may comprise any number of post-translational modifications. The peptides or any of the constituent amino acids may be processed, e.g., contacted with protecting groups, alkylated, beta-elimination of phosphate groups, etc., as is described elsewhere herein. In some instances, the peptides are derived from larger peptides or proteins and are fragmented.

Substrates: One or more operations described herein may be performed using a substrate. For example, one or more molecules described herein (e.g., polymeric analyte, capture moiety, polymerizable molecule) may be coupled to a substrate. In some instances, the polymeric analyte, capture moiety, and one or more polymerizable molecules (e.g., the first or second polymerizable molecule), or a combination thereof may be provided coupled to one or more substrates. In one example, the polymeric analyte, capture moiety, and the second polymerizable molecule are coupled to a substrate. In some instances, more than one substrate may be used. In such cases, the substrates may comprise the same material or different material.

The substrate may be made from any suitable material, e.g., glass, silicon, gel, polymer, etc., as is described elsewhere herein. In some instances, the substrate may be a bead or a gel bead (e.g., polyacrylamide, agarose, or TentaGel® bead). The substrate may be functionalized. One or more molecules, e.g., a capture moiety and the polymeric analyte (e.g., a peptide) may be coupled to the substrate via a covalent or non-covalent interaction. The capture moiety and polymeric analyte (e.g., peptide) can be coupled to the substrate using any suitable chemistry, e.g., click chemistry moieties (e.g., alkyne-azide coupling), photoreactive groups (e.g., benzophenone), 1-ethyl-3-(3-dimethylaminopropyl)carbodiimide hydrochloride (EDC) (e.g., to couple amino-oligos or peptides), N-hydroxysulfosuccinimide (NHS), Sulfo-NHS, or NHS-esters (e.g., to couple sulfhydryl oligos), maleimides, hydrazines, hydroxyl amines, thiols, biotin-streptavidin interactions, cystamine, glutaraldehyde, formaldehyde, succinimidyl 4-(N-maleimidomethyl)cyclohexame-1-carboxylate (SMCC), Sulfo-SMCC, 4-(4,6-Dimethoxy-1,3,5-triazin-2-yl)-4-methylmorpholinium chloride (DMTMM), silane (e.g., amino silanes), combinations thereof, etc. In some instances, the substrate may be functionalized to comprise a coupling chemistry to couple the polymeric analyte or the capture moiety. In one non-limiting example, a substrate (e.g., bead or surface) may comprise an alkyne such as dibenzocyclooctyne (DBCO), which may be configured to react to an amine (e.g., DBCO-alcohol, DBCO-Boc, DBCO-NHS), a carboyxl or carbonyl (e.g., DBCO, DBCO-silane), a sulfhydryl, etc. An azide-functionalized nucleic acid or protein may react with DBCO to link the nucleic acid or protein to the DBCO substrate. In other examples, linkers such as bifunctional linkers may be used to attach a molecule to a substrate; such bifunctional linkers may comprise the same reactive moiety on both ends or a different moiety at each end (e.g., heterobifunctional linker).

In some instances, a molecule (e.g., polymeric analytes, capture moieties, polymerizable molecules) may be coupled to the substrate using an enzymatic approach, e.g., as described elsewhere herein. For example, a chemical linker or moiety such as a click chemistry moiety may be attached to a polymeric analyte (e.g., peptide) using an enzyme. The chemical linker or moiety may be able to react with another chemical linker or moiety (e.g., click chemistry moiety) of a substrate, capture moiety, or polymerizable molecule.

The substrates may be coupled to any useful number of molecules (e.g., polymeric analytes, capture moieties, polymerizable molecules). In some instances, a substrate may comprise a plurality of polymeric analytes, a plurality of capture moieties, and/or a plurality of polymerizable molecules, which may be provided at any useful ratio or density. For example, the ratio of polymeric analytes to capture moieties or polymerizable molecules may be about 1:1, 1:5, 1:10, 1:20, 1:100, 1:1000, 1:10,000, 1:100,000, 1:1,000,000 or lower. In some instances, the ratio of polymeric analytes to capture moieties or polymerizable molecules may be at most about 1:1, at most about 1:5, at most about 1:10, at most about 1:20, at most about 1:100, at most about 1:1000, at most about 1:10,000, at most about 1:100,000, at most about 1:1,000,000 or lower.

Similarly, the molecules (e.g., polymeric analytes, capture moieties, or polymerizable molecules) may be coupled to the substrate at any useful density, for example about 1 molecule/square micron (m²), about 10 molecules/μm², about 100 molecules/μm², about 1,000 molecules/μm², about 10,000 molecules/μm² , about 100,000 molecules/μm² , about 1,000,000 molecules/μm² , about 10,000,000 molecules/μm², about 100,000,000 molecules/μm², about 1,000,000,000 molecules/μm², about 10,000,000,000 molecules/μm², about 100,000,000,000 molecules/μm², or greater. The polymeric analytes, capture moieties, and polymerizable molecules may be coupled to the substrate at a range of densities, e.g., from about 100 to about 10,000 molecules/μm², or from about 10 to about 1,000 molecules/μm². The density of the polymeric analytes, capture moieties, and polymerizable molecules may be the same or different. For example, the density of the polymerizable molecules may be 1-fold, 2-fold, 3-fold, 4-fold, 5-fold, 6-fold, 7-fold, 8-fold, 9-fold, 10-fold, 100-fold, 1000-fold, 10,000-fold, 100,000-fold, 1,000,000-fold or greater-fold lower than that of the polymeric analyte.

In some instances, the molecules coupled to the substrate may be spaced apart at a designated or controlled distance. For example, the average spacing or distance between the polymerizable molecules coupled to the substrate may be about 1 nanometer (nm), about 2 nm, about 3 nm, about 4 nm, about 5 nm, about 6 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 500 nm, about 1 m or greater. In some instances, the spacing between the polymerizable molecules coupled to the substrate may be at most about 1 μm, at most about 500 nm, at most about 100 nm, at most about 90 nm, at most about 80 nm, at most about 70 nm, at most about 60 nm, at most about 50 nm, at most about 40 nm, at most about 30 nm, at most about 20 nm, at most about 10 nm, at most about 5 nm, or less. Similarly, the spacing or distance between a polymeric analyte and a polymerizable molecule or capture moiety may be about 1 nanometer (nm), about 2 nm, about 3 nm, about 4 nm, about 5 nm, about 6 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 500 nm, about 1 m or greater. In some instances, the average spacing between the polymerizable molecule and the polymeric analyte coupled to the substrate may be at most about 1 m, at most about 500 nm, at most about 100 nm, at most about 90 nm, at most about 80 nm, at most about 70 nm, at most about 60 nm, at most about 50 nm, at most about 40 nm, at most about 30 nm, at most about 20 nm, at most about 10 nm, at most about 5 nm, or less. A range of average distances between the polymerizable molecules from one another or from the polymeric analytes may be used, e.g., from about 1 nm to about 40 nm, from about 2 nm to about 10 nm, etc.

The concentration or density of the molecules attached to the substrate may be modulated using one or more suitable approaches, including patterning or random deposition approaches. Examples of methods to control the concentration or density of the molecules attached to the substrate include limited dilution, addition of chaotropes (e.g., guanidine, formamide, urea), using metal organic compounds, etc. The molecules may be attached to the substrate in a patterned fashion, e.g., using self-assembling monolayers, photopatteming, lithography, etching, or a combination thereof, or the molecules may be randomly arranged.

The substrate may comprise any useful size or dimension (e.g., length, width, height, diameter, radius), surface area, volume, or ratio or combination thereof. The substrate may comprise a bead or particle that may comprise a diameter of about 1 nanometer (nm), about 2 nm, about 3 nm, about 4 nm, about 5 nm, about 6 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 500 nm, about 1 m, about 2 m, about 3 m, about 4 m, about 5 m, about 6 μm, about 7 μm, about 8 am, about 9 am, about 10 am, about 20 am, about 30 am, about 40 am, about 50 m, about 60am, about 70 am, about 80 am, about 90 am, about 100 am, about 200 μm, about 300 am, about 400 am, about 500 am, about 600 am, about 700 am, about 800 μm, about 900 μm, about 1 millimeter (mm) or greater. The substrate may comprise a surface area of about 1 square nanometer (nm²), about 10 nm², about 100 nm², about 1,000 nm², about 10,000 nm², about 100,000 nm², about 1 m², about 10 m², about 100 m², about 1,000 m², about 10,000 μm², about 100,000 μm², about 1 mm², about 10 mm², about 100 mm², about 1,000 mm², about 10,000 mm², about 100,000 mm², about 1,000,000 mm² or greater.

The molecules may be coupled to the substrate in an ordered or random arrangement. In ordered arrangements, the molecules may be patterned using any conventional approach such as lithography (e.g., soft lithography, photolithography), etching (e.g., ion etching, photo etching), or other patterning approach. In some instances, a linker (e.g., bifunctional linker) may be used to facilitate the coupling of the molecules (e.g., polymeric analytes, polymerizable molecules, capture moieties) to the substrate; such linkers may be patterned using any useful technique such as self-assembling monolayers, photopatterning, lithography, etching. In some instances, the molecules may be coupled to the substrate in a random arrangement. For example, the molecules may be provided at a stoichiometric ratio or controlled concentration to couple the molecules at any useful ratio or density.

Polymerizable Molecules: The polymerizable molecules described herein may be any useful type of polymerizable molecule. The polymerizable molecules may by naturally occurring, such as biological polymers (e.g., nucleic acid molecules, peptides, polysaccharides, fatty acids), or other naturally occurring polymers, e.g., rubber, cellulose, starches, polyhydroxyalkanoates, chitosan, dextran, structural proteins (e.g., collagen, hyaluronic acid, glycosaminoglycans), agarose, carrageenan, isphagula, acacia, agar, gelatin, shellac, xanthan gum, guar gum, alginate, etc. The polymerizable molecules may be synthetic, e.g., acrylics, nylons, silicones, viscose, rayon, polyesters, polycarboxylic acids, polyvinyl acetate, polyacrylamide, polyacrylate, polyethylene glycol, polyurethane, polylactic acid, silica, polystyrene, polyacrylonitrile, polybutadiene, polycarbonate, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and combinations thereof. The polymerizable molecules may comprise one or more reactive moieties (e.g., radical groups) to initiate polymerization or may be polymerized via contacting of an initiating agent (e.g., ammonium persulfate, peroxide, or other radicalizing agent). The polymerizable molecules may be polymerizable via contacting of an enzyme (e.g., polymerizing enzyme such as polymerases), ribozyme or DNAzyme. Alternatively or in addition to, the polymerizable molecules may be polymerizable via self-assembly. The polymerizable molecules may comprise a single polymer type (e.g., a homopolymer) or more than one polymer type (e.g., a copolymer) and may comprise random or arranged monomers. The polymerizable molecules may be a block polymer, alternating copolymer, periodic copolymer, statistical copolymer, stereoblock copolymer, gradient copolymer, branched copolymer, graft copolymer, etc.

The same or different types of polymerizable molecules may be used in the methods described herein. For example, the first polymerizable molecule comprised by or coupled to the binding agent may be a nucleic acid molecule, and the second polymerizable molecule may be a peptide. In another example, both the first polymerizable molecule and the second polymerizable molecule are nucleic acid molecules. In such an example, the first polymerizable molecule may be coupled to the second polymerizable molecule via ligation or hybridization. For instance, the first polymerizable molecule may comprise a first nucleic acid sequence and the second polymerizable molecule may comprise a second nucleic acid sequence. The first nucleic acid sequence may be complementary or partially complementary to the second nucleic acid sequence, and the coupling may comprise hybridizing the first nucleic acid sequence or portion thereof to the second nucleic acid sequence or portion thereof Alternatively, the first nucleic acid sequence and the nucleic acid sequence may be complementary to two sequences of a splint or bridge oligonucleotide, and coupling may be mediated via hybridization to the splint oligo. The first nucleic acid sequence may be ligated to the second nucleic acid sequence, either chemically (e.g., via click chemistry approaches in which the first polymerizable molecule and the second polymerizable molecule comprise one member of a click chemistry pair) or enzymatically (e.g., using a ligase).

The polymerizable molecules may comprise functional portions. For example, the polymerizable molecules may comprise a nucleic acid molecule comprising a functional sequence, such as a primer sequence (e.g., universal priming site), a sequencing sequence, a read sequence, a unique molecular identifier (UMI), a barcode sequence, a cleavage sequence (e.g., a restriction site, a Cas-binding sequence), a transposition sequence (e.g., a mosaic end sequence), or a combination thereof.

The polymerizable molecules may be any useful size. The polymerizable molecules may be about 1 angstrom, about 2 angstrom, about 3 angstrom, about 4 angstrom, about 5 angstrom, about 6 angstrom, about 7 angstrom, about 8 angstrom, about 9 angstrom, about 10 angstrom, about 20 angstrom, about 30 angstrom, about 40 angstrom, about 50 angstrom, about 60 angstrom, about 70 angstrom, about 80 angstrom, about 90 angstrom, about 100 angstrom, about 200 angstrom, about 300 angstrom, about 400 angstrom, bout 500 angstrom, about 600 angstrom, about 700 angstrom, about 800 angstrom, about 900 angstrom, about 1000 angstrom, about 10,000 angstrom, about 100,000 angstrom or greater in size, length, or another dimension. In some instances, the polymerizable molecule (e.g., the first polymerizable molecule or the second polymerizable molecule) comprises a nucleic acid molecule comprising one or more nucleotide bases. The polymerizable molecule may comprise any useful number of nucleotide bases, e.g., about 1 base, about 2 bases, about 3 bases, about 4 bases, about 5 bases, about 6 bases, about 7 bases, about 8 bases, about 9 bases, about 10 bases, about 20 bases, about 30 bases, about 40 bases, about 50 bases, about 60 bases, about 70 bases, about 80 bases, about 90 bases, about 100 bases, about 200 bases, about 300 bases, about 400 bases, about 500 bases, about 600 bases, about 700 bases, about 800 bases, about 900 bases, about 1000 bases, or a greater number of bases.

The polymerizable molecules may comprise a nucleic acid molecule. The nucleic acid molecule can be single stranded, double stranded, or partially double-stranded. The nucleic acid molecule may comprise a modified nucleotide or non-canonical base. For instance, the polymerizable molecules may comprise a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof. In some instances, a polymerizable molecule may comprise a hexitol nucleic acid (HNA) or a cyclohexyl nucleic acid (CeNA), which may be useful in rendering the polymerizable molecule more resistant to acid degradation (e.g., as used in conventional Edman degradation). Alternatively or in addition to, a polymerizable molecule may comprise naturally occurring bases that are more resistant to acid degradation, e.g., be composed of primarily thymine or cytosine. For example, a nucleic acid molecule may comprise at least 50%, at least 60%, at least 70%, at least 80%, at least 90%, or 100% thymines or cytosines, which can render the nucleic acid molecule more acid resistant as compared to a nucleic acid molecule comprising adenines or guanines.

Sequencing Reagents: One or more operations of the method may be mediated using a sequencing reagent provided herein, e.g., a sequencing reagent of Formulas I, IV, IV′, IV″, IV-A, IV-B, or V-C. In some instances, the coupling of the monomer to the capture moiety to generate a monomer-capture moiety complex is mediated using a sequencing reagent. The coupling of the sequencing reagent to the monomer or capture moiety may be covalent or noncovalent. In an example, a sequencing reagent may comprise a first reactive group that is able to couple to a monomer of the polymeric analyte (e.g., an amino acid of a peptide) and optionally, cleave the amino acid from a peptide. In some embodiments, the first reactive group is a guanidinylating agent. Other examples of amino acid reactive groups include isothiocyanate (ITC) such as phenyl isothiocyanate (PITC), 3-pyridyl isothiocyanate (PYITC), 2-piperidinoethyl isothiocyanate (PEITC), 3-(4-morpholino) propyl isothiocyanate (MPITC), 3-(diethylamino)propyl isothiocyanate (DEPTIC) or naphthylisothiocyanate (NITC), ammonium thiocyanate, potassium thiocyanate, trimethylsilyl isothiocyanate (TMS-ITC), phenyl phosphoroisothiocyanatidate, acetyl isothiocyanate (AITC), or an aldehyde group, e.g., ortho-phthalaldehyde (OPA), 2,3-naphthalenedicarboxyaldehyde (NDA), or 2-pyridinecarboxyaldehyde.

The sequencing reagent may additionally comprise a capture-binding moiety comprising a second reactive group that is capable of coupling, either directly or indirectly, to the capture moiety. In an example of direct coupling, the capture moiety may comprise a click chemistry moiety (e.g., alkyne), and the second reactive group of the sequencing reagent may comprise an additional click chemistry moiety (e.g., azide) that can react with the click chemistry moiety of the capture moiety. Alternatively, the sequencing reagent may be coupled indirectly to the capture moiety, e.g., via noncovalent interaction or via an intermediate linking molecule. In one such example, the intermediate linking molecule comprises a linking polymerizable molecule (e.g., a polymer or nucleic acid molecule) that can couple the sequencing reagent to the capture moiety. In one such example, the linking polymerizable molecule comprises (i) a third reactive group that is capable of coupling (e.g., via click chemistry) to the second reactive group of the sequencing reagent and (ii) a moiety that can couple to the capture moiety (e.g., another orthogonal click chemistry reaction, avidin-biotin interaction, nucleic acid coupling or hybridization). In some instances, the linking polymerizable molecule comprises a nucleic acid molecule that comprises (i) a click chemistry moiety (e.g., alkyne) that can conjugate to the second reactive group (e.g., azide) of the sequencing reagent and (ii) a nucleic acid sequence that acts as a capture-binding moiety and can couple to the capture moiety, e.g., via ligation, splint ligation, or hybridization. Accordingly, the coupling of the linking polymerizable molecule to the sequencing reagent may yield a sequencing reagent that is indirectly linked to a nucleic acid sequence which acts as the capture-binding moiety. In some embodiments, the sequencing reagent comprises the linking polymerizable molecule comprising the capture-binding moiety.

When applicable, the click chemistry moieties of the sequencing reagent and capture moiety or intermediate linking molecule may comprise any suitable bioorthogonal moieties, as described elsewhere herein, e.g., alkenes, alkynes, azides, epoxides, amines, thiols, nitrones, isonitriles, isocyanides, aziridines, activated esters, and tetrazines, and combinations, variations, or derivatives thereof The sequencing reagent may be subjected to conditions sufficient to react the first click chemistry moiety to the second click chemistry moiety, e.g., provision of metal catalysts, appropriate solvents, pH, temperature, ionic concentration, or light/energy for any useful duration of time.

The first reactive group of the sequencing reagent may be an amino acid-reactive moiety. The amino acid- reactive moiety of the sequencing reagent may be any useful moiety that enables the reactive moiety to conjugate to and optionally cleave an amino acid. In some examples, the first reactive moiety can react with a terminal amino acid (e.g., NTAA or CTAA). In such examples, the first reactive moiety may comprise any primary amine or carboxylic group reactive group, including but not limited to guanidinylating agents, isocyanates, acyl azides, NHS esters, sulfonyl chlorides, aldehydes, glyoxals, epoxides, oxiranes, carbonates, aryl halides, imidoesters, carbodiimides, anhydrides, phenyl esters, isothiocyanates (e.g., phenyl isothiocyanate, sodium isothiocyanate, ammonium isothiocyanates (e.g., tetrabutylammonium isothiocyanate, tetrabutylammonium isothiocyanate), diphenylphosphoryl isothiocyanate), acetyl chloride, cyanogen bromide, carboxypeptidases, azide, alkyne, DBCO, maleimide, succinimide, thiol-thiol disulfide bonds, tetrazine, TCO, vinyl, methylcyclopropene, acryloyl, allyl, among others. Additional examples of amino acid reactive groups are provided in U.S. Pat. Pub. No. 2020/0217853, which is incorporated by reference herein in its entirety. In some instances, the first reactive group comprises a guanidinylating agent.

The sequencing reagent may comprise any additional useful moieties. For example, the sequencing reagent may comprise a releasable or cleavable moiety, which may facilitate removal of the monomer from the polymeric analyte, or portion thereof, or from the substrate. For example, the sequencing reagent may comprise Formula I (e.g., or IV, IV′, IV″, IV-A, IV-B, or V-C), and the releasable or cleavable moiety may be comprised within L¹ or the capture-binding moiety. Such a releasable or cleavable moiety may comprise, for example, a disulfide bond, which may be releasable by contacting with a reducing agent (e.g., DTT, TCEP). In some examples, the sequencing reagent may couple to a linking polymerizable molecule via the releasable or cleavable moiety, alternatively or in addition to the coupling via click chemistry moieties. As such, the coupling between the linking polymerizable molecule and the sequencing reagent may be reversible. The sequencing reagent may additionally or alternatively comprise any number of spacing moieties, e.g., polymers (e.g., PAG, PEG, PPG, PVA, polyacrylamide), aminohexanoic acid, nucleic acids, alkyl chains, etc. Such spacing moieties may increase the distance between any other moieties of the sequencing reagent, e.g., the amino acid-reactive group and capture-binding moiety.

Use of a sequencing reagent comprising two reactive groups may allow for coupling of the sequencing reagent to (i) the monomer of the polymeric analyte (e.g., NTAA) and (ii) the intermediate linking molecule or (iii) the capture moiety. In some instances, when an intermediate linking molecule is used, the sequencing reagent may be pre-coupled to the intermediate linking molecule. For example, a precursor sequencing reagent may comprise a monomer-binding group (e.g., guanidinylating agent) and a click chemistry moiety (e.g., azide), which may be reacted with a polymerizable molecule (e.g., oligonucleotide) comprising complementary click chemistry moiety (e.g., alkyne) to generate a sequencing reagent that is capable of coupling to the monomer and the capture moiety (e.g., another oligonucleotide). In some instances, the sequencing reagent may be provided pre-coupled to the intermediate linking molecule.

FIG. 2 schematically shows an example sequencing reagent that may be used in sequencing polymeric analytes such as peptides. FIG. 2 Panel A shows a bifunctional sequencing reagent 203 (e.g., 1-(but-3-yn-1-yl)-4-isothiocyanatobenzene) comprising an amino acid reactive moiety (e.g., PITC) and an alkyne click chemistry moiety, which may be reacted with a polymerizable molecule 201 (e.g., a linking nucleic acid molecule) comprising a complementary azide click chemistry moiety. The bifunctional sequencing reagent may also comprise a spacer moiety, e.g., an alkyl chain (an ethyl group is depicted) of any length, a polymer (e.g., PEG) of any length, etc. The spacer moiety may be located between the amino acid reactive moiety and the click chemistry moiety. FIG. 2 Panel B shows the product of a click chemistry cycloaddition reaction between the azide and alkyne groups to generate a sequencing reagent molecule comprising the polymerizable molecule and the amino acid reactive moiety. The conjugation of the polymerizable molecule 201 to the bifunctional sequencing reagent 203 may occur at any useful or convenient step. In alternative examples (not shown), the bifunctional sequencing reagent 203 may comprise an azide group, e.g., 1-(2-azidoethyl)-4-isothiocyanatobenzene, which can be reacted to a polymerizable molecule 201 comprising an alkyne moiety.

Capture Moieties: The capture moieties may couple to a monomer of the polymeric analyte via any suitable mechanism. The coupling of the monomer to the capture moiety may comprise a covalent interaction or a noncovalent interaction. The coupling may occur by interaction of binding pairs, e.g., biotin and avidin (or streptavidin), cyclodextrins and small hydrophobic molecules (e.g., alkanes, benzene, polycyclics), cucurbiturils and adamantaneammonium or trimethylammoniomethyl ferrocene, cyclophane (e.g., calixarenes, cavitands, pillararenes, tetralactams), etc.

In some instances, the capture moiety comprises an additional polymerizable molecule (e.g., a nucleic acid molecule). In such instances, the monomer may be first coupled with a complementary polymerizable molecule (e.g., to generate a peptide-oligonucleotide conjugate) and tethered to the capture moiety, e.g., via complementary base pairing directly or via a splint molecule. Alternatively, the monomer may be coupled to the capture moiety via a linker, as is described above. For example, the linker may comprise a monomer-coupling group (e.g., PITC, which can couple to or react with an amino acid of a peptide) and a nucleic acid molecule. The capture moiety may comprise an additional nucleic acid molecule, which may be coupled to the nucleic acid molecule of the linker via hybridization, ligation, or both.

The capture moiety may comprise a nucleic acid molecule, which can comprise any naturally occurring, non-naturally occurring or engineered nucleotide base. For example, the nucleic acid molecule may comprise a pseudo-complementary base, a bridged nucleic acid, a xenonucleic acid, a locked nucleic acid, a peptide nucleic acid (PNA), a gamma-PNA, a morpholino, etc., as is described elsewhere herein.

The capture moiety may comprise one or more functional sequences, including, but not limited to a priming sequence, sequencing sequence, sequencing read sequence, a mosaic end sequence, a transposase recognition sequence, a cleavage site (e.g., restriction site), a UMI, a blocking group, a spacer sequence, a barcode sequence, or other functional sequence. In some instances, the capture moiety comprises a cleavable or releasable moiety (e.g., a restriction enzyme recognition site, an abasic site, a uracil which can be cleaved using USER® or uracil DNA glycosylase, a disulfide bond that can be releasable upon addition of a reducing agent).

In some instances, the capture moiety and a polymerizable molecule are provided coupled to a substrate. In one example, the substrate comprises, coupled thereto, a first nucleic acid molecule, a second nucleic acid molecule, and a capture moiety, which may be a third nucleic acid molecule. In some instances, a substrate may comprise identical nucleic acid molecules across the substrate; these identical nucleic acid molecules may act as both a capture moiety and a polymerizable molecule to which additional polymerizable molecules (e.g., coupled to binding agents) may couple. Alternatively, or in addition to, the capture moiety may be coupled to the polymeric analyte.

The capture moiety may comprise any useful moiety or functional group. The capture moiety may have a monomer-capture group, a substrate-tethering group or linker, or any additional functional groups or moieties, e.g., for coupling or tethering to other molecules or for detection. In some examples, the capture moiety comprises a nucleic acid molecule that comprises a substrate-tethering group (e.g., biotin, a click chemistry moiety such as an azide) that can couple to a substrate (e.g., comprising streptavidin or a complementary click chemistry). The capture moiety may additionally comprise a binding sequence, to which another nucleic acid molecule (e.g., a linking nucleic acid molecule. A linker-monomer complex. Or a binding agent nucleic acid barcode molecule) can couple, e.g., via hybridization, ligation, or both. In some instances, the capture moiety comprises a single-stranded oligonucleotide or a single-stranded region in which a complementary oligonucleotide can hybridize. The complementary oligonucleotide may comprise a detectable label (e.g., fluorophore) that allows for detection of the capture moiety.

Cleaving: The cleaving of the monomer from the polymeric analyte may be achieved using any suitable mechanism, such as via application of a stimulus. The stimulus can be, for example, a chemical stimulus, a biological stimulus, a thermal stimulus (e.g., application of heat), a photo-stimulus, a physical or mechanical stimulus, or other type of stimulus or a combination of stimuli. In some instances, the stimulus may be a chemical stimulus, e.g., a change in pH, addition of a lytic agent, initiating agent, radical-generating agent, reducing agent, etc. In some instances, the stimulus may be a biological stimulus, e.g., enzyme (e.g., Edmanase, protease, endonuclease, artificial protease such as artificial peptidase) that can cleave or catalyze cleavage of the monomer from the polymeric analyte.

In some examples, the polymeric analyte comprises a peptide and the monomer comprises an amino acid (e.g., an NTAA, CTAA, or internal amino acid). The method may comprise using a sequencing reagent comprising an amino acid reactive group (e.g., PITC) by coupling the amino acid reactive group of the linker with the amino acid, and cleaving the amino acid from the peptide using a stimulus (e.g., change in pH, temperature). In an example, PITC may couple to an NTAA under mildly alkaline conditions to generate a phenylthiocarbamoyl (PTC) derivative of the NTAA, and cleavage of the NTAA from the peptide may be achieved using an Edman degradation reaction (e.g., application of an acid such as trifluoroacetic acid with heat), to generate a thiazolinone (ATZ) derivative or a phenylthiohydantoin (PTH) derivative. As described elsewhere herein, the sequencing reagent may comprise a moiety or molecule (e.g., nucleic acid molecule or polymerizable molecule) that can also couple to the capture moiety such that, subsequent to cleavage, the cleaved amino acid may be coupled to the capture moiety.

In some instances, more than one monomer may be cleaved from the polymeric analyte at a time. The cleaving may comprise cleaving 2 monomers, 3 monomers, 4 monomers, 5 monomers, 6 monomers, 7 monomers, 8 monomers, 9 monomers, 10 monomers, or more. For example, the polymeric analyte may comprise a peptide comprising a plurality of amino acid monomers, and single amino acids, di-peptides, tri-peptides, quadri-peptides, or larger may be cleaved in the methods described herein. In some instances, at most about 10 monomers, at most about 9 monomers, at most about 8 monomers, at most about 7 monomers, at most about 6 monomers, at most about 5 monomers, at most about 4 monomers, at most about 3 monomers, or fewer monomers may be cleaved in a given cleavage event. In some instances, cleavage of greater than one monomer (e.g., amino acid) may be mediated using an enzyme (e.g., Edmanase, protease) that is capable of recognizing or cleaving more than a single amino acid.

Cleavage of the monomer (or plurality of monomers) may be conducted using a biological stimulus, such as an enzyme. The enzyme can be any useful cleaving enzyme, e.g., a protease, such as an Edmanase, cruzain, x protein (e.g., ClpS, ClpX), Proteinase K, exopeptidase, aminopeptidase, diaminopeptidase, serine protease, cysteine protease, threonine protease, aspartic protease, aspartic protease, glutamic protease, metalloprotease, asparagine peptide lyase, pepsin, trypsin, pancreatin, Lys-C, Glu-C, Asp-N, chymotrypsin, carboxypeptidase (e.g., carboxypeptidase A, carboxypeptidase B, carboxypeptidase Y), SUMO protease, elastase, papain, endoproteinase, proteinase, TrypZean®, bromelain, collagenase, hyaluronase, thermolysin, ficin, keratinase, tryptase, fibroblast activation, enterokinase, chymotrypsinogen, chymase, clostripain, calpain, alpha-lytic protease, proline specific endopeptidase, furin, thrombin, subtilisin, genenase, PCSK9, cathepsin, prolidase, methionine aminopeptidase, cathepsin C, 1-cyclohexen-1-yl-boronic acid pinacol ester, pyroglutamate aminopeptidase, renin, kininogen, kallikrein, DPPIV/CD26, thimet oligopeptidase, prolyl oligopeptidase, leucine aminopeptidase, dipeptidylpeptidase, or other enzyme or protease, or a combination or variation (e.g., engineered mutant or variant) thereof. In some instances, the cleaving enzyme or ribozyme or DNAzyme may be configured or engineered to cleave a terminal monomer or plurality of monomers; alternatively, the cleaving enzyme or ribozyme or DNAzyme may be configured or engineered to cleave off-site at a non-terminal location of the polymeric analyte, e.g., at an internal monomer within the polymeric analyte, at an n-1, n-2, n-3, n-4, n-5, n-6, n-7, n-8, n-9, n-10, etc. position (where n is the number of monomers in the polymeric analyte).

In the instances of enzymatic cleavage, additional reagents may be provided to catalyze or induce the cleavage. For instance, metalloproteases, aminopeptidases, or exopeptidases may facilitate cleavage of an amino acid or plurality of amino acids in the presence of a catalyst, e.g., metal or metal ion (e.g., cobalt). Accordingly, a catalyst may be provided in order to facilitate the binding of the enzyme to an amino acid or the subsequent cleavage of the amino acid from the peptide. In some examples, cleavage may be mediated by an apo-enzyme, which is inactive in the absence of a metal catalyst of cofactor, and cleavage may be controlled by addition of metal or metal ions.

Other examples of cleaving stimuli may include: a photo stimulus (e.g., application of UV, X-rays, gamma rays, or other wavelength of light), mechanical stimulus (e.g., sonication, high pressure), thermal stimulus (e.g., application of heat), or chemical stimulus. In some instances, the polymeric analyte may comprise or be altered to comprise a cleavable or labile bond that can be cleaved upon application of the appropriate stimulus, e.g., disulfide bonds (e.g., cleavable upon application of a chemical stimulus such as a reducing agent), ester linkages (e.g., cleavable with a change of pH), a vicinal-diol linkage (e.g., cleavable with sodium periodate), a Diels-Alder linkage (e.g., cleavable upon application of heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)).

Modifications of Monomers: In some instances, one or more monomers of the polymeric analyte may be modified. Modifications may be naturally-occurring (e.g., post translational modifications) or non-naturally occurring, such as by labeling or tagging, e.g., with an amino acid- or amine-reactive agent such as an isothiocyanate (e.g., PITC, NITC), 1-fluoro-2,-4-dinitrobenzene (DNFB), dansyl chloride, 4-sulfonyl-2-nitrobfluorobenzene (SNFB), an acetylating agent, an acylating agent, an alkylating agent, a guanidination agent, a thioacetylation agent, a thioacylation agent, a thiobenzoylation agent, or a derivative or combination thereof. Alternatively, or in addition to, the one or more monomers may be modified to comprise any useful moiety such as an adduct (e.g., a polymer such as PEG, a polymerizable molecule such as a nucleic acid molecule, a nanoparticle or nanotube, a peptide or protein), a lipid, a carbohydrate, a metabolite, a fluorophore, a hapten, a quencher, a tag (e.g., a fluorescent tag, a magnetic tag, a radioactive tag), a barcode, or other moiety. In some instances, a monomer of the polymeric analyte may be modified to facilitate recruitment of an enzyme to recognize or cleave a terminal monomer (e.g., a NTAA or CTAA of a peptide, the 5′ or 3′ nucleotide of a nucleic acid molecule, or the first or last monomer of a polymer) or set of monomers. For example, a terminal amino acid of a peptide analyte may be modified with a saccharide in order to recruit a lectin or lectin-bound protease. In another example, one or more monomers of a polymeric analyte may comprise or be coupled to a nucleic acid molecule having a first sequence that is complementary to a second sequence comprised by an oligo-bound protease. Hybridization of the first sequence to the second sequence may facilitate local recruitment of the protease to the monomer to be cleaved. In yet another example, a peptide analyte may be modified with PITC, which may allow for recruitment and cleavage by an Edmanase. In some examples, modifications to monomers of a polymeric analyte may include epitope tags, which can facilitate binding of a binding agent (e.g., subsequent to cleavage of the monomer from the polymeric analyte). Examples of such epitope tags include fluorophores, nucleic acid molecules, peptides, haptens, polymers, chemical moieties, or other adduct molecule. Additional examples of modifications to polymeric analytes are described elsewhere herein.

The polymeric analyte may comprise one or more modified monomers. The modification of the monomers may be naturally occurring, or synthetic. Synthetic modifications may be performed prior to, during, or subsequent to cleavage of a monomer from the polymeric analyte and may be advantageous in preserving the identity of the monomer. For instance, during standard Edman degradation reactions to cleave a terminal amino acid (monomer) from the peptide, some amino acid residues may be altered or rendered undetectable by the reaction conditions. In an example, the conditions of Edman degradation may cause oxidation of cysteine residues, dehydration or destruction of the phenylthiohydantoin (PTH) forms of serine or threonine, react with and modify lysine residues, or render some post-translational modifications undetectable. As such, it modifying a peptide prior to analysis, e.g., to protect some of the amino acid residues or post-translational modifications may be useful in more accurately identifying each of the amino acid residues. In an example of a modification that can be performed prior to cleavage, a peptide or portion thereof may be alkylated, e.g., to alkylate the cysteine residues (e.g., using 4-vinylpyridine, iodoacetamide, iodoacetate, chloroacetate); acetylated, e.g., to react serine or threonine residues form an ester (e.g., using acetyl chloride) or using acetic anhydride; oxidized, e.g., to convert cysteine residues to cysteic acid; reduced (e.g., using a reducing agent such as dithiothreitol, P-mercaptoethanol, or TCEP); contacted with a protecting group, e.g., phosphorylated residues may be protected (e.g., using a P-elimination of a phosphate group, with an optional Michael addition of a thiol group, e.g., as described in Knight, et al. Nat. Biotechnology. 21, 1047-1054 (2003), which is incorporated by reference herein in its entirety), etc. The polymeric analyte or monomer may be modified with a protecting group or moiety, such as a methyl, formyl, ethyl, acetyl, t-butyl, anisyl, benzyl, tifluoroacetyl, N-hydroxysuccinimide, t-butyloxycarbonyl (Boc), benzoyl, 4-methyl benzyl, thioanizyl, thiocresyl, benzyloxymethyl, 4-nitrophenyl, benzyloxycarbonyl, 2-nitrobenzoyl, 2-nitrophenylsulphenyl, 4-toluenesulphonyl, pentafluorophenyl, diphenylmethyl, 2-chlorobenzyloxycarbonyl, 2,4,5-trichlorophenyl, 2-bromobenzyloxycarbonyl, 9-fluorenylmethyloxycarbonyl (FMOC), triphenylmethyl, or 2,2,5,7,8-pentamethyl-chroman-6-sulphonyl group. The polymeric analyte or monomer may be treated with a protecting agent, e.g., carboxyethyl methanethiosulfonate (CEMTS), thiazolidine, mercaptophenyl acetic acid, cyanobenzothiazole (e.g., for lipidation of N-terminal cysteines), acetamidomethyl, 2-methylsulfonylethyl-oxycarbonyl, etc. In some instances, the lysine residues may be blocked (e.g., the primary amines of lysine residues may be reacted) using an isothiocyanate (e.g., PITC), and optionally carrying out a single round of Edman degradation to generate a new N-terminal exposed end.

In some instances, a monomer of the polymeric analyte may be modified to facilitate cleavage of the monomer from the polymeric analyte. For example, an amino acid monomer of a peptide polymeric analytic may be modified such that it is recognized by an enzyme, e.g., acetylation of an amino acid, which can facilitate acyl peptide hydrolase cleavage of the acetylated amino acid. Additional or alternative modifications to the monomers, such as those described herein, may also facilitate recognition by or interaction with an engineered cleaving enzyme.

In some instances, a monomer comprising a naturally-occurring modification may be treated to remove or alter the naturally-occurring modification to render the polymeric analyte or monomer more amenable to the processing operations disclosed herein. For example, acetylation, formylation, methylation, and pyrrolidone carboxylic acid post-translational modifications may be removed prior to sequencing. Acetylation modifications may be removed with acyl peptide hydrolase or acid treatment (e.g., using 1N HCl). Methylation may be removed using aminopeptidases. Formylation modifications may be removed, for example, using acid treatment (e.g., 0.6M HCl treatment). Pyrrolidone carboxylic acid (PCA) may be removed with pyroglutamate aminopeptidase. Exemplary C-terminal modifications may include amidation and methylation, both of which may be removed using carboxypeptidases.

Binding agents: The binding agent may be contacted with the monomer (e.g., subsequent to cleavage and monomer-capture moiety coupling). The binding agent may be any useful molecule that can couple to the monomer or monomer-capture moiety complex. For example, a binding agent may be or comprise a protein or peptide (e.g., an antibody, antibody fragment, single chain variant fragment (scFv), nanobody, anticalin, tRNA synthetase or tRNA-acyl synthetase, a fibronectin domain), a peptide mimetic, a peptidomimetic (e.g., a peptoid, a beta-peptide, a D-peptide peptidomimetic), a polysaccharide, a nucleic acid molecule (e.g., aptamer), a somamer, a polymer, an inorganic compound, an organic compound, a small molecule, or derivatives (e.g., engineered variants) or combinations thereof. In instances where the polymeric analyte comprises a peptide, the binding agent may be able to bind to a modified amino acid (e.g., an amino acid coupled to a linker) or portion thereof The binding agent may comprise a recognition site that specifically recognizes an amino acid, modified amino acid (e.g., an amino acid bound to a linker comprising a PITC moiety), or a derivatized (and optionally modified) amino acid. For example, the binding agent may be configured to recognize or have binding specificity to a moiety of a modified amino acid, such as a specific amino acid residue, the residue-linker complex, or derivatized amino acid (e.g., a thiocarbomyl-derivatized residue, a thiazolone-derivatized residue, a thiohydantoin-derivatized residue, etc.), or a portion of a modified amino acid. In some instances, the binding agent may be derived or engineered from a naturally-occurring enzyme or protein, e.g., an aminopeptidase, exopeptidase, metalloprotease, antibody, anticalin, N-recognin protein, Clp protease, endoprotease (e.g. trypsin), or tRNA synthetase. In some examples, a binding agent may be a cleaving enzyme (e.g., trypsin, endoprotease) that has been modified to remove the peptidase activity. The binding agent may also recognize a terminal amino acid that is attached to a substrate; for example, after all but the final monomer of a polymeric analyte has been coupled to the capture moiety or capture moieties and cleaved, the final monomer may remain coupled to a substrate. Accordingly, the binding agent may recognize and bind the surface-coupled monomer.

The binding agents may be contacted with and specifically bind to cleaved monomers, monomer-linker complex, monomer-linker-capture moiety, or monomer-capture moiety complexes (altogether referred to herein as “monomeric analytes”). For example, a monomeric analyte may fall in any size or range of sizes that is less than that of the entire polymeric analyte. A monomeric analyte complex may be about 0.1 nanometer (nm), about 0.5 nm, 1 about 1 nm, about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1 micrometer (m), about 10 m, about 100 m, about 1 millimeter mm in size or greater. The monomeric analyte may have any molecular weight or range of molecular weights. The monomeric analyte may be about 1 dalton (Da), 10 Da, 100 Da, 500 Da, 1 kilodalton (kDa), 10 kDa, 100 kDa, 1,000 kDa, 10,000 kDa, 100,000 kDa, or greater. The monomeric analyte may vary in molecular weight or length, e.g., depending on the amino acid residue.

The binding agent may comprise or be coupled, directly or indirectly, to a polymerizable molecule. The polymerizable molecule may be the same type of molecule as the binding agent (e.g., both peptides, both nucleic acid molecules, etc.), or they may be different. In some instances, the binding agent comprises a peptide (e.g., antibody or antibody fragment) and the polymerizable molecule comprises a nucleic acid molecule. The polymerizable molecule may be conjugated to the binding agent via a chemical conjugation approach, e.g., using linkers such as SMCC, (N-e-maleimidocaproyloxy)succinimide ester (EMCS), succinimidyl-4-(p-maleimidophenyl)butryate (SMPB), succinimidyl-(N-maleimidopropionamido-ethyleneglycol) ester (SMPEG), Succinimidyl (NHS) esters, succinimidyl-4-formylbenzamide (S-4FB), succinidmidyl-6-hydrazino-nicotinamide (S-HyNic), 4-Phenyl-3H-1,2,4-triazoline-3.5(4H)diones (PTAD) or other diazonium, 1-ethyl-3-3-dimethylaminoproyl carbodiimide hydrochloride (EDC), etc. Synthesis of the peptide-nucleic acid molecule conjugate may also be carried out using solid-phase synthesis, fragment conjugation (e.g., using heterobifunctional crosslinkers such as those comprising an aliphatic chain and a maleimide group on one end and NHS on the other), click chemistry (e.g., strain-promoted azide alkyne cycloaddition, inverse-electron-demand Diels-Alder reactions), or combinations of approaches or chemistries. In some instances, the polymerizable molecule may be conjugated to the binding agent using an enzymatic approach. For example, a DNA-protein conjugate may be generated using a truncated a nuclease (e.g., Cas protein such as Cas9), a relaxase (e.g., VirD2), or other enzyme, ribozyme, or DNAzyme. In some instances, the polymerizable molecule may be conjugated to the binding agent using a SpyTag and SpyCatcher interaction, a biotin-avidin interaction, a SNAP-tag, or other interaction. Optional purification may be performed, e.g., using ion-exchange chromatography, HPLC, affinity chromatography, or other purification technique.

The binding agent may be coupled to the polymerizable molecule via a noncovalent interaction. For instance, the binding agent may comprise an avidin or streptavidin tag, to which biotin-conjugated polymerizable molecules can bind. Alternatively, the binding agent may comprise a biotin tag to which an avidin or streptavidin-conjugated polymerizable molecule can bind.

The polymerizable molecule may comprise identifying information of the binding agent. For example, the polymerizable molecule may comprise a nucleic acid barcode molecule comprising a barcode sequence. The barcode sequence may encode for the identity of the binding agent or the binding partner. For example, a monomer (e.g., amino acid) of a polymeric analyte (e.g. peptide comprising a plurality of amino acids) may be cleaved and coupled to the capture moiety (e.g., on a substrate) and may be contacted with a binding agent (e.g., antibody, antibody fragment, nanobody). The binding agent may specifically recognize the amino acid residue or derivative thereof (e.g., a PTH, PTC, ATZ derivatized form) over other amino acid residues or derivatives thereof The nucleic acid barcode molecule may comprise information that identifies the binding agent, which, due to the specificity of the binding agent to its target, may also identify the particular amino acid residue (or derivative).

The polymerizable molecule of the binding agent may comprise additional multiplexed information. For example, the polymerizable molecule, e.g., a nucleic acid molecule, may comprise sequences that encode cycle or other temporal information or spatial information. In one such example, an array of peptides and capture moieties may be provided on a substrate. The array may comprise a plurality of individually addressable units, in which each (or a subset of) individually addressable units of the array comprises a peptide to be analyzed and a capture moiety. The binding agents, and the polymerizable molecules comprised therein or coupled thereto, may comprise spatial information (e.g., spatial barcode sequences) which uniquely identify the individually addressable units and thus the location of the array. The polymerizable molecules may additionally comprise temporal information (e.g., a cycle barcode that indicates the round or iteration in which the binding agent or polymerizable molecule is provided). Subsequent sequencing of the polymerizable molecule may be used to reveal the spatial information (e.g., the originating location in the array of a peptide or amino acid). In some instances, the polymerizable molecule may comprise a unique molecular identifier (UMI), which may be used to determine the quantity of a given binding agent or monomer (e.g., amino acid) for a given peptide, substrate, array, or sample.

Alternatively, the binding agent that recognizes the monomer-capture moiety complex may not comprise or be coupled to a polymerizable molecule. In such cases, subsequent to the binding of the binding agent to the monomer-capture moiety complex, an additional molecule (e.g., a secondary binding agent) comprising a detectable label, e.g., fluorophore, radioisotope, mass tag, or an identifying polymerizable molecule (e.g., nucleic acid barcode molecule) may be contacted with and bind to the binding agent that is bound to the monomer-capture moiety complex. In some examples, the additional molecule comprises an identifying polymerizable molecule, and the identifying polymerizable molecule may be coupled to or transferred to the additional polymerizable molecule. In one non-limiting example, the binding agent comprises a primary antibody or antibody fragment that recognizes the monomer-capture moiety complex (e.g., terminal amino acid- linker-capture moiety complex) or portion thereof (e.g., the terminal amino acid, or the terminal amino acid-linker complex); subsequent to binding of the primary antibody or antibody fragment to the monomer-capture moiety complex or portion thereof, a secondary antibody or antibody fragment comprising or coupled to a polymerizable molecule (e.g., nucleic acid barcode molecule) is coupled to the primary antibody. The polymerizable molecule of the secondary antibody or antibody fragment may comprise information on the secondary antibody or antibody fragment, the primary antibody or antibody fragment, or other information. Transfer or coupling of the polymerizable molecule of the secondary antibody or antibody fragment to the additional polymerizable molecule can be mediated by any suitable technique, e.g., hybridization of nucleic acid molecules optionally mediated by a splint molecule, click chemistry, or association of high affinity molecules (e.g., streptavidin and biotin).

In some instances, the method may comprise contacting the monomer-capture moiety complex with a library of binding agents. The library of binding agents may comprise a plurality of binding agents that have specificity to different analytes. For example, the library of binding agents may comprise a plurality of binding agents that recognize different amino acids or derivatives thereof (e.g., derivatized amino acids such as the PTH, PTC, or ATZ forms), clusters of amino acids (e.g., dipeptides, tripeptides, etc.), or combinations of amino acids (e.g., amino acids with similar side chain groups). In one such example, a given binding agent may recognize and bind to more than one amino acid, optionally with different affinities or binding kinetics. A given binding agent may recognize and bind to a single amino acid, two different amino acids, three different amino acids, four different amino acids, etc. For instance, a given binding agent may bind to amino acids with similar residues, e.g., amino acids with positively- charged side chains (e.g., arginine, histidine, lysine), negatively-charged side chains (aspartic acid, glutamic acid), amino acids with polar uncharged side chains (e.g., serine, threonine, asparagine, glutamine), amino acids with hydrophobic side chains (e.g., alanine, valine, isoleucine, leucine, methionine, phenylalanine, tyrosine, trytophan), or a combination thereof Altogether, the library of binding agents may specifically recognize or bind to any number of different amino acids; for example, the library of binding agents may be configured to specifically bind to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different proteinogenic amino acids or derivatives thereof.

The library of binding agents may comprise any useful number of binding agents, each of which can have different binding specificities. For example, a first binding agent may recognize and one amino acid, and second binding agent may recognize two amino acids, and a third binding agent may recognize three amino acids. In another example, a first binding agent may recognize one amino acid, a second binding agent may recognize a different amino acid, and a third binding agent may recognize a plurality of amino acids. It will be appreciated that any number of binding agents may be used and that each binding agent may have specificity to one or more amino acids. Altogether, the library of binding agents may bind to all 20 proteinogenic amino acids or derivatives thereof, or a subset (e.g., 10 or more, 15 or more) of the amino acids.

A binding agent may be passivated prior to or during contact with the cleaved monomer. Passivation may be achieved using a blocking agent or solution, such as milk proteins (e.g., lactoglobulin, lactalbumin, lactoferrin, casein, whey, immunoglobulin, insulin, growth factors, osteopontin), albumin (e.g., bovine serum albumin), Tween 20, commercially available blocking solutions, or a combination thereof Alternatively, or in addition to, passivation of the binding agent may be performed using a polymer (e.g., polyethylene glycol), organic compound (e.g., oil, lipids), sugar, nanoparticle, inorganic compound, ion, etc.

Coupling of Polymerizable Molecules: The polymerizable molecules may be coupled to one another using any useful approach. Such coupling may comprise a covalent interaction or a noncovalent interaction (e.g., ionic interaction, hydrophobic interaction, van der Waals forces, etc.). In some instances, the first polymerizable molecule and the second polymerizable molecule comprise nucleic acid molecules and may be coupled via hybridization, ligation, or both. For instance, the first polymerizable molecule may comprise a first sequence that is complementary to a second sequence of the second polymerizable molecule, and the coupling may occur via hybridization of the first sequence to the second sequence. Alternatively, the first sequence and the second sequence may not be complementary to one another, but may be complementary to a third sequence and a fourth sequence, respectively, of a splint or bridge oligonucleotide. Accordingly, coupling of the first polymerizable molecule to the second polymerizable molecule may be mediated by hybridization of the first and second sequences to the third and fourth sequences, respectively, of the splint or bridge oligonucleotide.

In some instances, a nucleic acid reaction may be performed as part of or in addition to the coupling of the first polymerizable molecule to the second polymerizable molecule. For example, the first sequence of the first polymerizable molecule may hybridize to the second sequence of the second polymerizable molecule, and a nucleic acid extension reaction (e.g., using a polymerase) may be performed. Such an extension reaction may allow for transfer of the encoded information of one of the polymerizable molecules (e.g., the first polymerizable molecule) to another polymerizable molecule (e.g., the second polymerizable molecule). In another example, the first sequence of the first polymerizable molecule may be ligated to the second sequence of the second polymerizable molecule to provide a first polymerizable molecule covalently coupled to the second polymerizable molecule.

The polymerizable molecules may be coupled chemically, either covalently or noncovalently. In some instances, the first polymerizable molecule may be chemically linked to the second polymerizable molecule. For example, the first polymerizable molecule may comprise a first reactive moiety, and the second polymerizable molecule may comprise a second reactive moiety that is capable of reacting with the first reactive moiety. The first reactive moiety may be contacted with the second reactive moiety and be subjected to conditions sufficient to link the first reactive moiety to the second reactive moiety, e.g., via click chemistry. In other instances, the first polymerizable molecule may be coupled to the second polymerizable molecule via a noncovalent or indirect interaction, e.g., biotin-streptavidin.

In some instances, the polymerizable molecule of the binding agent can be coupled to additional polymerizable molecules. For example, a substrate may comprise the polymeric analyte and capture moiety coupled thereto, along with a plurality of additional polymerizable molecules. After coupling of the monomer to the capture moiety and cleavage of the monomer from the polymeric analyte, the monomer may be contacted with the same or different binding agents any number of times. The polymerizable molecule of a single binding agent may contact and be coupled to any number of the additional polymerizable molecules iteratively for repeated interrogation; for instance, the polymerizable molecule of the binding agent may couple to a first additional polymerizable molecule, as described herein, and then subsequently cleaved or removed (e.g., via dehybridization) and contacted and coupled to a second additional polymerizable molecule. Such an approach may be advantageous in transferring several copies of the polymerizable molecule of the binding agent to the substrate. Decoupling Monomers, Binding Agents: In some instances, subsequent to the coupling of the first polymerizable molecule to the second polymerizable molecule, the monomer may be decoupled from the monomer-capture moiety complex or the substrate. The decoupling may be performed chemically, mechanically, or enzymatically. For example, in some instances, the monomer is coupled to the capture moiety via a linking nucleic acid molecule (e.g., a linker comprising a monomer reactive group and a linking nucleic acid molecule). The linking nucleic acid molecule may comprise a cleavage site, e.g., restriction site, and the decoupling may be performed by enzymatic cleavage at the cleavage site, using, for example, a restriction endonuclease. Alternatively, or in addition to, the capture moiety or any of the polymerizable molecules may comprise a cleavage site which may allow for decoupling of the monomer from a portion of the capture moiety. Other examples of enzymatic cleavage include, in non-limiting examples, glycosylases (e.g., uracil glycosylase), restriction endonucleases, micrococcal nucleases, transposases, Cas proteins (e.g., Cas9), Argonaut endonucleases, etc. Beneficially, the removal of the monomer from the monomer-capture moiety complex can allow the capture moiety to be available for subsequent reactions or iterations or prevent binding of additional binding agents to the capture moiety, which may help reduce erroneous or duplicative coupling of the polymerizable molecule of the additional binding agents to the capture moieties. Alternatively, or in addition to, the decoupling may occur using a stimulus, e.g., a photo-stimulus (such as UV, gamma, X-ray irradiation), thermal stimulus, chemical stimulus, etc. In some instances, the linker may comprise a cleavable group and application of the appropriate stimulus may result in cleaving of the linker, as described elsewhere herein.

Alternatively, or in addition to, the monomer may be altered such that it is rendered undetectable by the binding agent, e.g., to prevent binding of the binding agent to the cleaved monomer in subsequent iterations or cycles of cleaving, coupling to the capture moiety, and contacting with additional binding agents. For example, the monomer may be contacted with a blocking agent or derivatized such that the binding agent no longer recognizes the derivatized form. Such blocking strategies may be useful in eliminating the need to remove cleaved monomers following detection or transfer of information from the binding agent-coupled polymerizable molecules. Additional strategies for inhibiting binding of binding agents to cleaved monomers are described elsewhere herein.

Similarly, in some instances, the binding agent may be removed from the monomer-capture moiety complex at any useful or convenient operation, e.g., subsequent to coupling of the polymerizable molecules. Removal of the binding agent may be performed using chemical or enzymatic approaches, e.g., using chemical denaturants, detergents, acidic or alkaline conditions, heat, or proteases. Alternatively, or in addition to, if a polymerizable molecule is coupled to the binding agent, the polymerizable molecule may be removed from the binding agent, e.g., via a cleavage or restriction site and use of a cleaving enzyme (e.g., UDG, restriction enzyme), chemical cleavage, photolysis, or other approach. In some instances, the polymerizable molecule is coupled to the binding agent via a noncovalent interaction, e.g., desthiobiotin-avidin; accordingly, decoupling of the polymerizable molecule from the binding agent may be achieved by use of a competition agent, e.g., a higher-affinity biotin to competitively replace the desthiobiotin.

Identification of polymerizable molecules: The polymerizable molecules may be subjected to sequencing to determine the identity of the individual monomers (e.g., amino acids). For example, following cleavage of the monomer from the capture moiety (e.g., as shown in FIG. 1A Panel F), or any number of iterations of workflow 100, the polymerizable molecules comprising information of the binding agents, and thus the identity of the monomers, may be removed from the substrate and prepared for sequencing (e.g., DNA sequencing, NGS). Removal of the polymerizable molecules may be accomplished using any useful approach, e.g., chemical or enzymatic cleavage. In some instances, any excess or uncoupled polymerizable molecules or capture moieties may be removed, e.g., prior to removal of the polymerizable molecules comprising monomer information. For example, referring again to FIG. 1A Panel B and FIG. 1A Panel E, the coupling events may generate double-stranded or partially-double-stranded molecules; accordingly, the single-stranded polymerizable molecules or capture moieties that do not comprise a monomer or additional polymerizable molecules (e.g., from the binding agents) may be digested using an enzyme such as type II restriction endonucleases, S1 endonucleases.

Alternatively, or in addition to, the polymerizable molecules may be amplified (e.g., using nucleic acid amplification approaches such as polymerase chain reaction (PCR), isothermal amplification, ligation-mediated amplification, transcription-based amplification, etc.) to generate amplicons for sequencing. Amplification may be performed, for example, using the capture moieties or polymerizable molecules as primer binding sites. Any number of useful preparation operations may be performed, such as purification or enrichment, cleanup, nucleic acid reactions (e.g., ligation, extension, amplification, tagmentation, restriction enzyme cleavage), fragmenting, barcoding, addition of adapters, enzymatic treatment, etc. In some instances, the polymerizable molecules, or the substrates comprising the polymerizable molecules, may be filtered based on any useful characteristic or properties. Filtering based on a characteristic or property may achieve higher accuracy or less noise by removing poor quality molecules or enriching for higher quality polymerizable molecules prior to sequencing. For example, polymerizable molecules or substrates (e.g., beads or particles) containing the polymerizable molecules may be filtered by size or length, quantity, presence of particular sequences (e.g., primer sequences, sequences of interest), GC content, polarity, polarization, birefringence, fluorescence (or other optical property), anisotropy, charge, secondary structure (e.g., hairpins), or other useful metric, characteristic, or property or combinations thereof. Such filtration or enrichment may be performed using any suitable approach, e.g., affinity or hybridization approaches (e.g., bead based affinity sequences or hybridization assays, which can enrich particular sequences), chromatography, size-based filtration, electrophoresis, electrofocusing, optoelectronics, digital fluidics, magnetic activated sorting, fluorescence activated sorting, flow cytometry, or other suitable technique.

Sequencing may be performed using a commercially available nanopore system, e.g., Oxford Nanopore Technologies, Genia Technologies, NobleGen, or Quantum Biosystem, or other sequencing and next generation sequencing systems, e.g., Illumina, BGI, Qiagen, ThermoFisher, PacBio, and Roche, including formats such as parallel bead arrays, sequencing by synthesis, sequencing by ligation (e.g., SOLiD), capillary electrophoresis, electronic microchips, “biochips,” microarrays, parallel microchips, single-molecule arrays, and Sanger sequencing, as is described elsewhere herein.

Sequencing may output the identity of the polymerizable molecules or sequences of polymerizable molecules that are coupled together. For example, referring again to FIG. 1A, subsequent to one or more iterations of workflow 100, the polymerizable molecule 107 may comprise a nucleic acid sequence of the polymerizable molecule 117 of the binding agent (or complement thereof), or stacks of polymerizable molecules obtained from multiple rounds of binding agents binding to their target monomer or monomer-capture moiety complex (or complements thereof). Sequencing of the polymerizable molecule 107 may therefore yield sequencing reads that identify the nucleic acid sequence of the polymerizable molecule 117 of the binding agent and the information encoded therein, e.g., the cycle number and the identity of the binding agent or monomer (e.g., one of the 20 proteinogenic amino acids). In instances where the polymerizable molecule 117 of the binding agent comprises a nucleic acid molecule that encodes additional information (e.g., comprises barcode sequences, UMIs, cycle information, spatial information etc.), multiple types of information may be revealed from the nucleic acid sequencing of the polymerizable molecule 107.

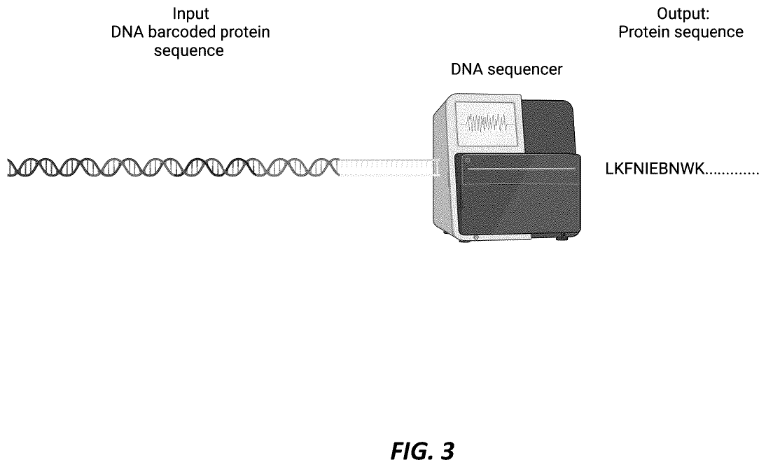

FIG. 3 schematically shows a diagram of inputting, into a sequencing instrument, barcoded DNA sequences (e.g., a stacked polymerizable molecule resulting from multiple iterations of workflow 100 of FIG. 1A) and outputting a peptide or protein sequence. The barcoded DNA sequences comprise stacks of barcodes sequences obtained from individual binding agents that bind to their target monomer or monomer-capture moiety complex; each barcode sequence encodes for the identity of the monomer (e.g., amino acid).

Sequencing reads may be assembled using a de novo approach to identify the peptide or protein. For instance, fragmented peptides arising from a common parent protein may be labeled with a common barcode sequence, as described elsewhere herein. Putative peptide reads can thus be assembled based on the common barcode sequence, amino acid identity, and if applicable, cycle number. Erroneous reads may be identified through probabilistic modeling of accuracy of reads, resulting in reconstructed, fragmentary, peptide sequences (contigs) with possible gaps for missed or unidentified rounds/amino acid. An alternative option for de novo read reconstruction may employ end-to-end, unsupervised machine learning based reconstruction of peptide reads. This option may employ a Machine Learning Algorithm, which refers to a deep-learning based model that takes as its input NGS sequencing reads associated with a parent protein/peptide barcode, and outputs the likely reconstruction of peptide reads (contigs). Training of the model can be conducted with protein sequencing runs using known protein/peptide standards. The de novo reconstruction may output reconstructed, fragmentary, peptide sequences (contigs) with a probability assigned to each amino acid as well as the assembled peptide sequence. In some instances, a k-mer or De Brujin approach may be used for peptide sequence reconstruction. For example, reads arising from each polymerizable molecule may be broken down into shorter k-mer sequences. The k-mer sequences from the pool of reads may be assembled into longer contig sequences. A De Brujin graph may be generated, e.g., to represent splice variants, post-translational modifications, or other proteoforms. The isoforms may be assembled and the expression level may be determined using a Bayesian approach. The assembled isoforms of proteins may be subjected to evaluation and error correction, e.g., by comparison with standard proteins that are spiked in samples, and assessing for missing segments of sequences, incorrect or redundant assembly, uniform coverage, etc.

Alternatively, the identity of the polymerizable molecules may be obtained without use of a sequencing approach. For instance, probes may be used to couple to particular regions of a polymerizable molecule. The probes may comprise nucleic acid probes with probe sequences that can be used to specifically detect a type of monomer. In one such example, the polymeric analyte comprises a peptide, and an individual amino acid (monomeric unit) may be coupled to a capture moiety and cleaved from the peptide. The monomer-capture moiety complex may be contacted with a binding agent (e.g., antibody, nanobody, scFv) comprising a nucleic acid barcode molecule (polymerizable molecule) that identifies the binding agent. The binding agent may be specific to one amino acid (e.g., of the 20 proteinogenic amino acids) and as such, the nucleic acid barcode molecule encodes for one specific amino acid. Accordingly, a nucleic acid probe having a complementary sequence to the nucleic acid barcode molecule of the binding agent may be used to identify the presence of the binding agent (e.g., via in situ hybridization). In some instances, the probes may comprise detectable labels or moieties, e.g., a fluorophore, radioisotope, mass tag, etc. For example, hybridization-based assays such as SeqFISH or Nanostring may be performed to probe or assay particular regions of a polymerizable molecule to determine its identity. In other examples, an amplification based approach may be used to determine the presence and identity of a polymerizable molecule. For example, PCR or nested PCR approaches may be used to selectively probe for a particular sequence of a polymerizable molecule.

Alternatively or in addition to, the binding agent may comprise a detectable label or moiety. For example, the binding agent may comprise a fluorophore, radioisotope, mass tag, chromogenic enzyme (e.g., horse radish peroxidase), etc., which may be detectable using the appropriate imaging technique. Different binding agents (e.g., binding agents that recognize different monomers or amino acids) may be labeled with distinct labels, e.g., different fluorophores, which can be used to identify the presence of the monomer or amino acid. In some examples, fluorophore-labelled binding agents can be detected using single molecule imaging (e.g., total internal reflection, confocal, wide-field, or super resolution microscopy (e.g., PALM, STORM, STED)).

In some instances, the substrates comprising the polymerizable molecules (e.g., following one or more iterations of workflow 100 of FIG. 1A) may be provided on an array for sequencing. For example, a plurality of beads comprising polymerizable molecules that encode for amino acids of a plurality of peptides may be provided on an array for sequencing. In one such example, the plurality of beads may be directly or indirectly coupled to an additional substrate (e.g., planar substrate, such as microscope slides or multi-well plates), and sequencing may be performed using image-based sequencing approaches (e.g., using sequencing by synthesis or in situ hybridization probes and a single-molecule resolution imaging system), amplification-based sequencing, or both. The plurality of beads may be coupled to the additional substrate using any suitable technique such as nucleic acid attachment using the polymerizable molecules or capture molecules, magnetic attachment (using a magnetic field and magnetic beads), optoelectronics, digital microfluidics, application of an electric field, gravity settling, centrifugation, capillary force, hydrogen bonding, electrostatic interactions or other suitable approach.

Fingerprinting: The methods described herein may be useful in complete de novo protein or peptide sequencing (e.g., the identification of each amino acid in a peptide), or for fingerprinting a protein (e.g., identifying only a subset of amino acid types in a peptide and inferring, using a reference database, the identity of the peptide). For fingerprinting, a subset of amino acids may be identified, e.g., using the approaches described herein, without the need of binding agents that are specific to all 20 proteinogenic amino acids. For example, 1, 2, 3, 4, 5, 6, 7, 8, 9, 10, 11, 12, 13, 14, 15, 16, 17, 18, or 19 different binding agents with single-amino acid or multi-amino acid specificity may be sufficient to determine the identity of a protein or peptide. For known proteome databases, reference-based reconstruction may be performed by simulating NGS reads that would be generated from the set of possible peptides in the workflow. For each possible peptide, a simulation can produce NGS reads mimicking the output of this protein sequence system. Next, the real (experimental) NGS reads from a run can be matched to simulated reads from candidate peptides from a database based on likelihood. This results in reconstructed, fragmentary, peptide sequences (contigs) with probability assigned to the assembled peptide sequence.

High Throughput Sequencing Parallelization: The methods described herein may be conducted in a parallelized, high-throughput format. Such parallelization may be achieved by having substrates comprising multiple polymeric analytes coupled thereto and performing the operations (e.g., coupling a monomer to the capture moiety, cleaving, contacting with a binding agent or library of binding agents, coupling of polymerizable molecules, optional cleavage of the monomer from the capture moiety) iteratively, across the substrate. In some instances, a library of binding agents may be used to recognize different monomer types (e.g., different amino acids of a peptide analyte or derivatized amino acids), such that different polymeric analytes (e.g., different peptides) may be processed on a single substrate.

A library of binding agents may be used to recognize different monomer types to facilitate high-throughput readout. As described herein, the library of binding agents may comprise binding agents that can recognize a single monomer (e.g., a single cleaved amino acid) or multiple monomers (e.g., multiple cleaved amino acids). In some instances, binding agents with varying levels of specificity may be used in a sequence or order, which may help render a less-specific binding agent to be more specific, simply based on the sequence in which it is provided. For example, a first binding agent may be capable of specifically binding to a first monomeric analyte, and a second binding agent may be capable of binding to both the first monomeric analyte and a second monomeric analyte. The first binding agent may be provided and contacted with the first monomeric analyte and second monomeric analyte. Since the first binding agent is specific to the first monomeric analyte, the first binding agent will bind exclusively to the first monomeric analyte. Subsequently, the second binding agent may be provided; however, since the first monomeric analyte is bound to the first binding agent, the first monomeric analyte may be inaccessible (e.g., sterically blocked) to the second binding agent. As such, the second binding agent may bind only to the second monomeric analyte. Accordingly, identification of the first binding agent and second binding agent (e.g., through detection of a label/tag or through sequencing of polymerizable molecules coupled to the binding agents and optionally transferred to a substrate), may allow for identification of the first monomeric analyte and the second monomeric analyte.

In some instances, it may be useful to barcode the polymeric analytes prior to processing. Barcode sequences may be attached to the polymeric analytes at a single location (e.g., at a terminus), multiple locations, adjacent to the polymeric analyte (e.g., on a substrate), etc. as is described elsewhere herein. For example, a peptide may be labeled at the N-terminus, C-terminus, or an internal amino acid with a nucleic acid barcode molecule. The nucleic acid barcode sequence may comprise information or be unique to a partition or compartment, sample, peptide, etc. such that each unique barcode sequence can be traced back (e.g., subsequent to nucleic acid sequencing or other detection method) to the originating partition or compartment, sample, peptide, etc.

Alternatively, or in addition to, the capture moieties or polymerizable molecules may comprise a barcode sequence. The barcode sequence may be specific to a particular partition, sample, or spatial location. For example, a substrate may comprise a plurality of individually or by introduction of chaotropic agents (e.g., guanidine, formamide, urea) units. The polymerizable units or capture moieties of each individually addressable unit may comprise a unique barcode specific to the individually addressable unit (e.g., a spatial barcode). The polymeric analytes may be coupled to the substrate such that each individually addressable unit comprises, on average, no more than one polymeric analyte. Such a distribution of polymeric analytes may be obtained, for example, using a limited dilution approach (e.g., diluting the polymeric analytes to reduce the number of polymeric analytes that may attach to a given individually addressable unit) or by introduction of chaotropic agents (e.g., guanidine, formamide, urea). The polymeric analytes may be distributed across the individually addressable units according to a Poisson distribution. Thus, for a given substrate, about 6%, 10%, 18%, 20%, 30%, 36%, 40%, or 50% of the individually addressable units may comprise one or fewer polymeric analytes.

Modifications of Polymeric Analytes and Polymerizable Molecules: The present disclosure also provides for methods of modifying polymeric analytes or monomers of the polymeric analytes (e.g., amino acids of a peptide), as well as polymerizable molecules described herein. Such modifications may useful, for example, in rendering a monomer more resistant to certain reaction conditions (e.g., Edman degradation), to increase or decrease binding affinity of a binding agent to the modified monomer, to assist in docking or interfacing of the modified monomer to an enzyme (e.g., a protease, cleaving enzyme or enzyme analog such as a ribozyme or DNAzyme, binding agent), or other purpose.

A polymeric analyte such as a peptide may be modified in order to render the peptide or a constituent amino acid more resistant to the reaction conditions for cleaving the amino acid from the peptide. For example, the peptide may be subjected to alkylation, e.g., using 4-vinylpyridine, iodoacetamide, which may be useful in preventing oxidation of cysteine residues. The peptide may be subjected to acetylation, e.g., 0-acetylation to form an ester such as acetyl chloride, which may be useful in preventing dehydration, racemization, or destruction of a derivatized (e.g., PTH form) of serine or threonine. The peptide may be subjected to P-elimination of a phosphate, followed by a Michael addition of a thiol group, (e.g., as described in Knight et al. 2003. Nature Biotechnology 21, 1047-1054, which is incorporated by reference herein) to detect phosphorylation events. The peptide may be contacted with phenyl isothiocyanate, acetic anhydride, or other amine-reactive group to protect lysine residues. Additional examples of peptide processing for Edman degradation can be found in Tarr, Methods of Protein Microcharacterization. Pp 155-194, which is incorporated by reference herein.

A polymeric analyte or monomer may be modified to influence the interaction of a binding agent with the polymeric analyte or monomer, e.g., by derivatizing the cleaved monomer, adding of chemical groups to the cleaved monomer, or other chemical processing (e.g., addition or removal of groups) from the cleaved monomer. Blocking of the binding agent may be achieved by appending a blocking agent, e.g., a chemical group or adduct, to the monomer-capture moiety complex; for example, conjugation a synthetic polymer (e.g., PEG), nucleic acid molecule, fluorophores, quenchers, nanotube, nanoparticle, small molecules, polypeptide or protein, fatty acid chain, or other large, sterically-hindering molecules. The blocking agents may be appended to the monomer using a chemical approach (e.g., reacting with an amino acid, e.g., via a photo-reaction) or enzymatically, e.g., using methyltransferases, tRNA synthetases, acetyltransferases, etc.

Additional examples of modifications to monomers, cleaved monomers, and binding agents can be found in International Patent Publication WO/2023196642, which is incorporated by reference herein in its entirety.

Order of Operations: It will be appreciated that the operations presented in the methods described herein may be performed in any useful or convenient order and that some operations, in some instances, may be optional. For example, in some instances, the coupling of the monomer to the capture moiety may occur prior to, during, or subsequent to the cleaving of the monomer from the polymeric analyte. Similarly, the coupling of the sequencing reagent to the capture moiety may occur prior to, during, or subsequent to the coupling of the sequencing reagent to the monomer. In another example, the substrate may be provided with the cleaved monomers coupled thereto, such that cleavage of the monomer from the polymeric analyte is obviated. In yet another example, in instances where a sequencing reagent is used to couple to the monomer (e.g., amino acid) and the capture moiety or substrate, the sequencing reagent may comprise a monomer-coupling group and subsequently be reacted with a capture-binding moiety (e.g., an oligonucleotide); alternatively, the sequencing reagent may be provided with the capture-binding moiety as part of the sequencing reagent (e.g., pre-conjugated to the capture-binding moiety).

Additional operations may be performed at any useful or convenient step, e.g., prior to provision of the polymeric analyte (e.g., peptide) or subsequent to one or more of the processing operations (e.g., subsequent to coupling of the sequencing reagent, polymerizable molecules, contacting with binding agents, etc.). For instance, it may be useful to purify or enrich or purify a population of polymerizable molecules (e.g., subsequent to process 110 in FIG. 1B or process 106 in FIG. 1F). Such enrichment or purification can be performed using any useful technique, e.g., bead-based enrichment, immunoprecipitation, chromatography, solid-phase reverse immobilization (SPRI), electrophoresis, DNA purification, etc. In one such example, purification of a nucleic acid molecule may be performed using a bead comprising or coupled to a complementary sequence of the nucleic acid molecule and optionally, subsequent to capture, eluting the nucleic acid molecule. Similarly, a protein may be purified using a bead comprising an antibody that recognizes the protein or a portion of the protein.

### Substrate Conjugation

The present disclosure provides methods for coupling molecules (e.g., biomolecules such as nucleic acid molecules, peptides, lipids, carbohydrates, etc.) to a substrate. The substrate may be functionalized to allow for covalent or noncovalent coupling of the molecules to a substrate. The substrate may comprise any useful functional moiety, e.g., a reactive moiety. In a non-limiting example, a reactive moiety may comprise a click chemistry moieties, such as azide, alkyne, nitrone, alkene (e.g., a strained alkene), tetrazine, methyltetrazine, triazole, tetrazole, phosphite, phosphine, etc. A click chemistry moiety may be reactive in copper-catalyzed Huisgen cycloaddition or the 1,3-dipolar cycloaddition between an azide and a terminal alkyne, a Diels-Alder reaction (e.g., a cycloaddition between a diene and a dienophile), or a nucleophilic substitution reactions in which one of the reactive species is an epoxy or aziridine. A molecule that is to be coupled to a substrate may comprise a complementary click chemistry moiety to that of the substrate; for example, the substrate may comprise an alkyne moiety and the molecule to be coupled may comprise an azide moiety, which can react with the alkyne moiety of the substrate to generate a covalent linkage. In one such example, the substate may comprise dibenzocyclooctyne (DBCO) moieties to which azide-comprising molecules (e.g., azide-DNA, azide-polymers, azide- peptides) can react and conjugate.

The reactive moiety may comprise a photoreactive moiety that may be activated when exposed to a photostimulus (e.g., light such as UV or visible light). Examples of photoreactive moieties include aryl (phenyl) azides (e.g., phenyl azide, ortho-hydroxyphenyl azide, meta-hydroxyphenyl azide, tetrafluorophenyl azide, ortho-nitrophenyl azide, meta-nitrophenyl azide), diazirines, azido-methyl-coumarins, benzophenones, anthraquinones, diazo compounds, diazirines, psoralen, and analogs or derivatives thereof.

The reactive moiety may comprise a carboxyl-reactive crosslinker group, such as diazomethane, diazoacetyl, carbonyldiimidazole, carbodiimides (e.g., 1-ethyl-3-(3-dimethylaminopropyl)carbodiimide hydrochloride (EDC)), dicyclohexylcarbodiimide (DCC)), or an amine-reactive group (e.g., N-hydroxysulfosuccinimide (NHS), Sulfo-NHS, or NHS-esters). The reactive group may comprise a crosslinking agent, which may comprise an NHS group, an EDC group, a maleimide, a thiol, a cystamine, an aldehyde, a succinimidyl group, an expoxide, an acrylate. Examples of crosslinking agents include, for example, NHS (N-hydroxysuccinimide); sulfo-NHS (N-hydroxysulfosuccinimide); EDC (1-Ethyl-3-[3-dimethylaminopropyl]); carbodiimide hydrochloride; SMCC (succinimidyl 4-(N-maleimidomethyl)cyclohexane-1-carboxylate); sulfo-SMCC; DSS (disuccinimidyl suberate); DSG (disuccinimidyl glutarate); DFDNB (1,5-difluoro-2,4-dinitrobenzene); BS3 (bis(sulfosuccinimidyl)suberate); TSAT (tris-(succinimidyl)aminotriacetate); BS(PEG)5 (PEGylated bis(sulfosuccinimidyl)suberate); BS(PEG)9 (PEGylated bis(sulfosuccinimidyl)suberate); DSP(dithiobis(succinimidyl propionate)); DTSSP (3,3′-dithiobis(sulfosuccinimidyl propionate)); DST(disuccinimidyl tartrate); BSOCOES (bis(2-(succinimidooxycarbonyloxy)ethyl)sulfone); EGS (ethylene glycol bis(succinimidyl succinate)); DMA (dimethyl adipimidate); DMP (dimethyl pimelimidate); DMS (dimethyl suberimidate); DTBP (Wang and Richard's Reagent); BM(PEG)₂ (1,8-bismaleimido-diethyleneglycol); BM(PEG)3 (1,11-bismaleimido-triethyleneglycol); BMB (1,4-bismaleimidobutane); DTME (dithiobismaleimidoethane); BMH (bismaleimidohexane); BMOE (bismaleimidoethane); TMEA (tris(2-maleimidoethyl)amine); SPDP (succinimidyl 3-(2-pyridyldithio)propionate); SMCC (Succinimidyl trans-4-(maleimidylmethyl)cyclohexane-1-Carboxylate); SIA (succinimidyl iodoacetate); SBAP (succinimidyl 3-(bromoacetamido)propionate); STAB (succinimidyl (4-iodoacetyl)aminobenzoate); Sulfo-SIAB (sulfosuccinimidyl (4-iodoacetyl) aminobenzoate); AMAS (N-α-maleimidoacet-oxysuccinimide ester); BMPS (N-β-maleimidopropyl-oxysuccinimide ester); GMBS (N-γ-maleimidobutyryl-oxysuccinimide ester); Sulfo-GMBS (N-γ-maleimidobutyryl-oxysulfosuccinimide ester); MBS (m-maleimidobenzoyl-N-hydroxysuccinimide ester); Sulfo-MBS (m-maleimidobenzoyl-N-hydroxysulfosuccinimide ester); SMCC (succinimidyl 4-(N-maleimidomethyl)cyclohexane-1-carboxylate); Sulfo-SMCC (sulfosuccinimidyl 4-(N-maleimidomethyl)cyclohexane-1-carboxylate); EMCS (N—ε-malemidocaproyl-oxysuccinimide ester); Sulfo-EMCS (N—ε-maleimidocaproyl-oxysulfosuccinimide ester); SMPB (succinimidyl 4-(p-maleimidophenyl)butyrate); Sulfo-SMPB (sulfosuccinimidyl 4-(N-maleimidophenyl)butyrate); SMPH (Succinimidyl 6-((beta-maleimidopropionamido)hexanoate)); LC-SMCC (succinimidyl 4-(N-maleimidomethyl) cyclohexane-1-carboxy-(6-amidocaproate)); Sulfo-KMUS (N-κ-maleimidoundecanoyl-oxysulfosuccinimide ester); SPDP (succinimidyl 3-(2-pyridyldithio)propionate); LC-SPDP (succinimidyl 6-(3(2-pyridyldithio)propionamido) hexanoate); LC-SPDP (succinimidyl 6-(3(2-pyridyldithio)propionamido)hexanoate); Sulfo-LC-SPDP (sulfosuccinimidyl 6-(3′-(2-pyridyldithio)propionamido)hexanoate); SMPT (4-succinimidyloxycarbonyl-alpha-methyl-u(2-pyridyldithio)toluene); PEG4-SPDP (PEGylated, long-chain SPDP crosslinker); PEG12-SPDP (PEGylated, long-chain SPDP crosslinker); SM(PEG)₂ (PEGylated SMCC crosslinker); SM(PEG)4 (PEGylated SMCC crosslinker); SM(PEG)6 (PEGylated, long-chain SMCC crosslinker); SM(PEG)8 (PEGylated, long-chain SMCC crosslinker); SM(PEG)12 (PEGylated, long-chain SMCC crosslinker); SM(PEG)24 (PEGylated, long-chain SMCC crosslinker); BMPH (N—O-maleimidopropionic acid hydrazide); EMCH (N-E-maleimidocaproic acid hydrazide); MPBH (4-(4-N-maleimidophenyl)butyric acid hydrazide); KMUH (N-κ-maleimidoundecanoic acid hydrazide); PDPH (3-(2-pyridyldithio)propionyl hydrazide); ATFB-SE (4-Azido-2,3,5,6-Tetrafluorobenzoic Acid, Succinimidyl Ester); ANB-NOS (N-5-azido-2-nitrobenzoyloxysuccinimide); SDA (NHS-Diazirine) (succinimidyl 4,4′-azipentanoate); LC-SDA (NHS-LC-Diazirine) (succinimidyl 6-(4,4′-azipentanamido)hexanoate); SDAD (NHS—SS-Diazirine) (succinimidyl 2-((4,4′-azipentanamido)ethyl)-1,3′-dithiopropionate); Sulfo-SDA (Sulfo-NHS-Diazirine) (sulfosuccinimidyl 4,4′-azipentanoate); Sulfo-LC-SDA (Sulfo-NHS-LC-Diazirine) (sulfosuccinimidyl 6-(4,4′-azipentanamido)hexanoate); Sulfo-SDAD (Sulfo-NHS—SS-Diazirine) (sulfosuccinimidyl 2-((4,4′-azipentanamido)ethyl)-1,3′-dithiopropionate); SPB (succinimidyl-[4-(psoralen-8-yloxy)]-butyrate); Sulfo-SANPAH (sulfosuccinimidyl 6-(4′-azido-2′-nitrophenylamino)hexanoate); DCC (dicyclohexylcarbodiimide); EDC (1-ethyl-3-(3-dimethylaminopropyl)carbodiimide hydrochloride); glutaraldehyde; formaldehyde; and combinations or derivatives thereof.

Molecules may also be attached to substrates using linkers. The linkers can have any useful number of functional groups or reactive groups and may be uni-functional (having one functional group), bi-functional, tri-functional, quadri-functional, or comprise a greater number of functional groups. In some instances, a molecule (e.g., nucleic acid molecule, peptide, or polymer) may be attached to a substrate using a heterobifunctional linker. The heterobifunctional linker may comprise any useful functional group, as described herein. Non-limiting examples of heterobifunctional linkers include: p-Azidobenzyol hydrazide (ABH), N-5-Azido-2-nitrobenzoyloxysuccinimide (ANB-NOS), N-[4-(p-Azidosalicylamido)butyl]-3′-(2′-pyridyldithio) propionamide (APDP), p-Azidophenyl Glyoxal monohydrate (APG), Bis[B-(4-azidosalicylamido)ethyl]disulfide (BASED), Bis[2-(Succinimidooxycarbonyloxy)ethyl] Sulfone (BSOCOES), BMPS, 1,4-Di[3′-(2′-pyridyldithio)propionamido] Butane (DPDPB), Dithiobis(succinimidyl Propionate) (DSP), Disuccinimidyl Suberate (DSS), Discuccinimidyl Tartrate (DST), 3,3′-Dithiobis(sulfosuccinimidyl Propionate (DTSSP), EDC, Ethylene Glycol bis (succinimidyl succinate) (EGS), N-(E-maleimidocaproic acid hydrazide (EMCH), N-(E-maleimidocaproyloxy)-succinimide ester (EMCS), N-Maleimidobutyryloxysuccinimide ester (GMBS), Hydroxylamine-HCl, MAL-PEG-SCM, m-Maleimidobenzoyl-N-hydroxysuccinimide Ester (MBS), N-Hydroxysuccinimidyl-4-azidosalicylic acid (NHS-ASA), PDPH, N-Succinimidyl bromoacetate (SBA), SIA, Sulfo-SIA, Succinimidyl-4-(N-maleimidomethyl)cyclohexane-1-carboxylate (SMCC), Succinimidyl 4-(p-maleimidophenyl) Butyrate (SMPB), Succinimidyl-6-[B-maleimidopropionamido]hexanoate (SMPH), N-Succinimidyl 3-[2-pyridyldithio]-propionate (SPDP), Sulfo-LC-SPDP, N-(p-Maleimidophenyl isocyanate (PMPI), N-Succinimidyl(4-iodoacetyl) Aminobenzoate (SIAB), Sulfo-MBS, Sulfo-SANPAH, Sulfo-SMCC, Sulfo-DST, Sulfo-EMCS, Sulfo-GMBS, N-Hydroxysulfosuccinimidyl-4-azidobenzoate (Sulfo-HSAB), Sulfosuccinimidyl (4-azidophenyl)-1,3 dithio propionate (Sulfo-SADP), Sulfosuccinimidyl 2-(m-azido-o-nitrobenzamido)-ethyl-1,3′-dithio propionate (Sulfo-SAND), Sulfosuccinimidyl-2-(p-azidosalicylamido)ethyl-1,3-dithiopropionate (Sulfo SASD), Sulfo-SIAB, Sulfo-SMCC, Sulfo-SMPB, and the like.

More than one type of molecule may be coupled to the substrate. For example, a substrate may be coupled to nucleic acid molecules and peptides. Alternatively, a substrate may be coupled to only one type of molecule (e.g., only nucleic acid molecules, only peptides, only lipids, only carbohydrates, etc.). A substrate may be coupled to any useful combination of molecules, linkers, reactive moieties or functional groups, which may be coupled at any useful density, as described elsewhere herein. For example, a multifunctional linker may be used to attach both a nucleic acid barcode molecule and a peptide to the substrate. Alternatively, a substrate may comprise a linker and reactive sites; the linker may be used to attach one type of molecule (e.g., peptides or nucleic acid molecules), whereas the reactive sites may be used to attach another type of molecule (e.g., nucleic acid molecules or peptides).

The proximity of a molecule coupled to a substrate to its nearest neighbor (e.g., another molecule) may be controlled using a variety of approaches, e.g., self-assembling monolayers, patterning approaches, linking moieties, etc. In some instances, it may be advantageous to have two molecules in close proximity (e.g., two polymerizable molecules, such as a peptide and a nucleic acid molecule, or two nucleic acid molecules). For instance, with respect to the sequencing approaches described herein, capture moieties may be used to couple a monomer of a polymeric analyte, and subsequent to monomer cleavage, additional polymerizable molecules may be required to be in proximity to the capture moiety to allow for transfer of information encoded by polymerizable molecules of binding agents. The proximity of the molecules (e.g., capture moiety and polymerizable molecules) may be mediated using tethering molecules, such as nucleic acid molecule “staples” or multi-functional linkers.

Nucleic acid molecules may be coupled to a substrate by direct coupling. In such instances, the substrate or the nucleic acid molecules may comprise functional moieties that can interact. For example, the substrate and nucleic acid molecules may comprise a complementary click chemistry pair, e.g., alkyne and azide. In one such example, a substrate may comprise alkyne moieties (e.g., DBCO), which can be reacted with azide-functionalized nucleic acid molecules. The nucleic acid molecules may be reacted with the alkyne moieties in a click chemistry reaction to covalently link the substrate to the nucleic acid molecules. In another example, the substrate may comprise avidin or streptavidin moieties, to which biotinylated nucleic acid molecules may interact and bind non-covalently.

Alternatively, or in addition to, the nucleic acid molecules may be coupled to a substrate using a linker, e.g., as described elsewhere herein. The linker may comprise at least two functional groups (e.g., a heterobifunctional linker) that can couple to both the substrate and the nucleic acid molecules. In an example, the substrate may comprise an amine group, and alkyne-functionalized DNA primers (e.g., DBCO-DNA primers) may be attached using a linker such as azidoacetic acid NHS ester. In another example, amine-functionalized substrates may be coupled to azide-functionalized DNA primers using a DBCO-NHS ester or DBCO-PEG-NHS ester linker.

Similarly, peptides may be coupled to a substrate by direct coupling or by using a linker. A peptide may be coupled to a substrate at a terminus of the peptide (e.g., C terminus or N terminus), at an internal residue or amino acid of the peptide, or at multiple locations along the peptide. In examples of direct coupling, a peptide may be functionalized with a moiety that can interact with a moiety of the substrate (e.g., click chemistry pair, avidin-biotin). For example, the substrate and peptides may comprise a complementary click chemistry pair, e.g., alkyne and azide, or binding partners such as avidin and biotin. In one example of a click chemistry pair, a substrate may comprise alkyne moieties (e.g., DBCO), which can be reacted with azide-functionalized peptides. The peptides may be reacted with the alkyne moieties in a click chemistry reaction to covalently link the substrate to the peptides. In another example, the substrate may comprise avidin or streptavidin moieties, to which biotinylated peptides may interact and bind non-covalently.

Alternatively, or in addition to, the peptides may be coupled to a substrate using a linker, e.g., as described elsewhere herein. The linker may comprise at least two functional groups (e.g., a heterobifunctional linker) that can couple to both the substrate and the nucleic acid molecules. In an example, the substrate may comprise an amine group, and alkyne-functionalized peptides may be attached using a linker such as azidoacetic acid NHS ester. In another example, amine-functionalized substrates may be coupled to azide-functionalized peptides using a DBCO-NHS ester or DBCO-PEG-NHS ester linker. In yet another example, substrates comprising an amine group may be coupled to an azide-functionalized peptide using EDC and Sulfo-NHS.

A peptide may be functionalized with a functional moiety to enable attachment or coupling of the peptide to the substrate. The functional moiety may comprise a click chemistry moiety or other linking moiety and can be attached to the peptide at a peptide terminus (N-terminus or C-terminus), or at an internal amino acid. Chemical approaches to functionalize peptides can include C-terminal-specific conjugation (e.g., via C-terminal decarboxylative alkylation) using photoredox catalysis, e.g., as described by Bloom et al, Nature Chemistry 10, 205-211. 2018. And Zhang et al, ACS Chem. Biol. 2021, 16, 11, 2595-2603, each of which is incorporated by reference herein in its entirety, or amide coupling to an amine-functionalized surface. N-terminal attachment may comprise amide coupling of the N-terminus amine group to a carboxylic group functionalized surface, or using 2-pyridinecarboxaldehyde variants. Alternatively, or in addition to, functionalization of terminal ends of peptides may be achieved enzymatically, e.g., using carboxypeptidases or amidases for C-terminal functionalization (e.g., as described in Xu et al, ACS Chem Biol. 2011 Oct. 21; 6(10): 1015-1020; Zhu et al, Chinese Chemical Letters. 2018, Vol 29 Issue 7, Pages 1116-1118; and Zhu et al, ACS Catal. 2022, 12, 13, 8019-8026, each of which is incorporated by reference herein in its entirety), Sortase A, subtiligase, Butelase I, or trypsiligase. In some examples, ubiquitin ligase can be used to attach ubiquitin proteins with linker moieties to substrates. These linker moieties can then be used to chemically attach proteins to ubiquitin-coupled substrates. Internal amino acid residues may be coupled to substrates using, for example, amide coupling using EDC/NHS chemistry or DMT-MM to Glutamate or Aspartate residues, alkylation or disulfide bridge labeling of cysteines, or amide coupling to lysine residues.

A peptide may be treated prior to, during, or subsequent to coupling of the peptide to a substrate. In some examples, it may be advantageous to block or protect primary amines or carboxyl groups and optionally, de-block or de-protect the N-terminus primary amine or C-terminus carboxy group in order to facilitate attachment of the N-terminus or C-terminus to a substrate. In an example, single-point (e.g., C-terminal) selective attachment of peptides can be achieved by reacting the peptide with a sequencing reagent comprising an amine-reactive group (e.g., isothiocyanates such as PITC) and a reactive group (e.g., click chemistry group). The sequencing reagent can be, for example, PITC-conjugated click chemistry moieties such as PITC-azide, PITC-alkyne, optionally with spacer moieties in between, e.g., PITC-alkyl-azide, PITC-PEG-azide, PITC-alkyl-alkyne, PITC-PEG-azide). The sequencing reagent reacts with and “blocks” the primary amines (e.g., modifies lysines), including the N-terminus. Subsequent cleavage of the N-terminal amino acid (e.g., using an Edman reagent, such as acid), can be performed, and one of the remaining modified lysines may be attached to a substrate (e.g., using the click chemistry moiety coupled to the amine-reactive group). Optionally, the peptide may be treated with a protease, e.g., LysC, which cleaves peptides such that a remaining peptide has a C-terminal lysine and such that the remaining peptide comprises a primary amine only at the C-terminal lysine residue and the N-terminus; such a cleavage may be performed prior to reacting the amine-reactive group, e.g., as shown by Xie et al. Langmuir 2022, 38, 30, 9119-9128, which is incorporated by reference herein in its entirety.

Similarly, carboxylic groups can be reacted in a way to enable C-terminal or internal residue attachment. In an example of C-terminal conjugation, carboxyl groups may be labeled with a C-terminal sequencing reagent, such as isothiocyanate, when treated with an activating reagent (e.g., acetic anhydride) to generate a peptide-thiohydantoin (at the C-terminus) and “blocked” carboxyl groups on the aspartic acid and glutamic acid residues. The thiohydantoin may then be reacted to couple to a substrate. Alternatively, cleavage of the C-terminal amino acid via a single round of C-terminal sequencing degradation, or via a protease, exposes only a single reactive carboxylic group at the C-terminal amino acid. The single reactive C-terminal carboxylic group can then be used as a reactive moiety for a single attachment site.

In another approach, a peptide or protein can be attached via the N-terminus using the specific reactivities of the N-terminus amine group. Amine-based reactions, such as amide coupling, can be carried out at low pH where only the N-terminal amine group is active. In addition, 2-pyridinecarboxyaldehyde and variants can be used to react to the N-terminal amine group.

In some instances, a peptide may be conjugated to a substrate using a polymerization reaction, e.g., a free radical polymerization, such as using PEGylated peptides, methacrylamide-modified peptides, Michael-type addition of maleimide-terminated oligo-NIPAAM-conjugated peptides; photocrosslinking of azophenyl-conjugated peptides, or other polymerization reactions with monomer-conjugated peptides, e.g., as described by Krishna et al. Biopolymers. 2010; 94(1): 32-48, which is incorporated by reference herein in its entirety.

Alternatively, or in addition to, a peptide may be conjugated to a substrate via a substrate-bound linker. The substrate-bound linker may comprise any useful conjugation moieties or reactive groups. In some instances, the substrate-bound linker comprises a biomolecule (e.g., lipid, carbohydrate, peptide, nucleic acid). For example, the substrate-bound linker may comprise a nucleic acid molecule, and the peptide may be linked or conjugated to an additional nucleic acid molecule that is capable of coupling to the substrate-bound nucleic acid molecule, e.g., via hybridization, ligation, or both.

Multiple types of molecules may be attached to a substrate. The substrate may comprise, coupled thereto, any combination of molecules, including but not limited to: peptides, proteins (e.g., enzymes, antibodies, nanobodies, antibody fragments), nucleic acid molecules, lipids, carbohydrates or sugars, metabolites, small molecules, polymers, metals, viral particles, biotin, avidin, streptavidin, neutravidin, etc. The multiple types of molecules may be attached simultaneously to the substrate or in a sequential manner. For example, a substrate may be treated to conjugate nucleic acid molecules and subsequently treated to conjugate peptides, or alternatively, the substrate may be treated to conjugate peptides prior to the nucleic acid molecules.

A substrate, or portion thereof, may be subjected to conditions sufficient to passivate the substrate or portion thereof Passivation of a substrate may be useful for a variety of purposes, such as preventing nonspecific binding of binding agents, altering the surface density of a molecule (e.g., increasing the density of nucleic acid molecules or peptides), blocking reactive sites (e.g., blocking available click chemistry moieties subsequent to conjugation of the molecules on the substrate), etc. Passivation may be achieved using chemical approaches, e.g., deposition of blocking agents such as proteins (e.g., albumin), Tween-20, polymers, metals or metal oxides, or biochemical approaches, e.g., using metal microbes. Substrates comprising reactive moieties may also be passivated following molecule conjugation (e.g., coupling of nucleic acid molecules, peptides, etc.) by reacting any unreacted sites with an appropriate molecule. For example, a substrate comprising click chemistry moieties, e.g., DBCO beads, may be coupled to molecules of interest (e.g., polymerizable molecules, such as nucleic acid molecules, peptides) at a useful density using click chemistry (e.g., azide-nucleic acid molecules, azide-peptides). Unreacted sites may be passivated by providing and reacting complementary click-chemistry molecules, e.g., azide-polymers (e.g., PEG-azide), which may reduce downstream nonspecific interactions.

Substrate passivation may occur at any useful time or step. For instance, passivation to block unreacted DBCO sites may be performed prior to, during, or subsequent to conjugation of analytes or other molecules of interest (e.g., peptides and nucleic acid molecules). The passivation may be controlled by stoichiometry or densities of the passivating agent relative to the molecules of interest, or by physical approaches, e.g., photopatteming, self assembling monolayers, etc.

### Sample Processing

The present disclosure also provides systems, compositions, devices, and methods for processing samples. One or more methods for processing samples may comprise preparation of biological samples for analysis, which, in some instances, includes partitioning of cells for conducting single- cell analysis. A method for processing a biological sample may comprise extraction or isolation of one or more peptides or proteins from the biological sample for further processing and analysis, as is described elsewhere herein.

Preparation of Cell Suspensions for Single-Cell Analysis: The methods described herein may involve preparation of single cell suspensions from a biological sample. Single cell suspensions may be prepared from biological samples by dissociating cells and optionally, culturing them in a liquid medium. In some instances, biological samples comprise a liquid sample. For example, a biological sample may comprise a bacterial liquid culture, a mammalian liquid culture, a blood, plasma, or serum sample. Processing of such liquid samples may include centrifugation (e.g., to isolate cells), resuspension of cells in a suitable medium, such as Dulbecco's Phosphate Buffered Saline (DPBS), and optional culturing of the isolated cells.

A biological sample may comprise cultured cells, e.g., cell cultured in suspension, or cells adhered to a solid surface, such as petri dishes or tissue culture dishes. Cultured adherent cells samples may be treated to generate a cell suspension, e.g., via a protease such as trypsin, to detach the cells from the surface. A biological sample may comprise a tissue or biopsy sample. A tissue or biopsy sample may be processed mechanically or enzymatically to generate a cell suspension. Such processing may include sonication (mechanical treatment) or enzymatic treatment, such as the use of pronase, collagenase, hyaluronidase, metalloproteinases, trypsin, or other enzymes that digest extracellular matrix components. The dissociated cells can then be stored in a suitable buffer, such as DPBS.

Cell Sorting: A biological sample or a cell suspension may be subjected to sorting to isolate a cell of interest. Sorting may be performed to select or isolate a cell based on a quality or characteristic of the cell, e.g., expression of a protein target, size, deformability, fluorescence or other optical property, or other physical property of the cell. Sorting may accomplished using any number of approaches, e.g., using immunosorting (e.g., fluorescence activated cell sorting (FACS) or magnetic activated cell sorting (MACS)), electrophoretic approaches, chromatography, microfluidic approaches (e.g., using inertial focusing, cell traps, electrophoresis), acoustic sorting, optical sorting (e.g., optoelectronic tweezers), mechanical cell picking (e.g., using manual or robotic pipettes) or passive approaches (e.g., gravitational settling).

Partitioning: Cells of a biological sample or cell suspension may be partitioned into individual partitions such that at least a subset of the individual partitions comprises a single cell. The individual partitions may comprise a barcode molecule (e.g., fluorophore or set of fluorophores, nucleic acid barcode molecules, etc.). Barcode molecules may be unique to the partition, such that each individual partition comprises a different barcode sequence than other partitions. The barcode molecules may be loaded into the individual partitions at any useful ratio of barcode molecules to sample species (e.g., cells, proteins, nucleic acid molecules). The barcode molecules may be loaded into partitions such that about 0.0001, 0.001, 0.1, 1, 2, 3, 4, 5, 6, 7, 8, 9, 10, 20, 50, 100, 500, 1000, 5000, 10000, or 200000 barcodes are loaded per sample species. In some cases, the barcodes are loaded into partitions such that more than about 0.0001, 0.001, 0.1, 1, 2, 3, 4, 5, 6, 7, 8, 9, 10, 20, 50, 100, 500, 1000, 5000, 10000, or 200000 barcodes are loaded per sample species. In some cases, the barcodes are loaded in the partitions so that less than about 0.0001, 0.001, 0.1, 1, 2, 3, 4, 5, 6, 7, 8, 9, 10, 20, 50, 100, 500, 1000, 5000, 10000, or 200000 barcodes are loaded per sample species.

A partition may assume any useful geometry such as a droplet, a microwell, a solid substrate, a gel (e.g., a cell encapsulated in a gel bead), a bead, a flask, a tube, a spot, a capsule, a channel, a chamber, or other compartment or vessel. A partition may be part of an array of partitions, e.g., a droplet in a microfluidic device, a microwell of a microwell plate, a spot on a multi-spot array, etc.

Lysis, Permeabilization, and Analyte Extraction: Single cells (e.g., in partitions) may be processed to obtain one or more analytes contained therein. A method for processing a single cell may comprise lysing the cell to release the contents into the individual compartment or partition. Lysis may be performed using a detergent (e.g., Triton-X 100, sodium dodecyl sulfate, sodium deoxycholate, CHAPS), RIPA buffer, a change in temperature (e.g., elevated or lower temperature, freezing, freeze-thawing), enzymes, mechanical lysis (e.g., sonication, application of mechanical force), electrical lysis, or a combination thereof Lysis may be performed in the presence of protease inhibitors to prevent degradation or digestion of the proteins from the cell. The contents may optionally be further processed, e.g., subjected to purification or extraction, denaturation of proteins or peptides, enzyme or chemical digestion, etc. In some instances, the contents may be subjected to enzymatic digestion to remove nucleic acid molecules, e.g. using nucleases such as DNAse or RNAse. Alternatively or in addition to, a cell may be fixed (e.g., using a fixative) and/or permeabilized. Examples of fixatives include aldehydes (e.g., glutaraldehyde, formaldehyde, paraformaldehyde), alcohols (e.g., methanol, ethanol), acetone, acids (e.g., acetic acid, Davidson's AFA), oxidizing agents (e.g., osmium tetroxide, potassium dichromate, chromic acid, permanganate salts), Zenker's fixative, picrates, Hepes-glutamic acid buffer-mediated organic solvent protection effect (HOPE), or Karnovsky fixative. Cell permeabilization may be achieved mechanically (e.g., using sonication, electroporation, shearing) or chemically (e.g., using an organic solvent such as methanol or acetone or detergents such as saponin, Tween-20, Triton X-100).

Protein Processing: The biological sample (or single cell suspensions or partitioned cells) may be further processed to enable proteomic analysis. For example, de-aggregation of proteins in the sample may be performed, e.g., using chemical or mechanical approaches. Chemical de-aggregation methods can include but are not limited to: sodium dodecyl (SDS), Triton-X 100, 3-((3-cholamidopropyl) dimethylamminio)-1-proppanesulfonate (CHAPS), ethylene carbonate, or formamide. Mechanical de-aggregation methods can include but are not limited to: sonication or high temperature treatment. The biological sample (or single cell suspensions or partitioned cells) may be subjected to conditions sufficient to denature one or more proteins. Denaturation may be achieved using heat, chemicals (e.g., SDS, urea, guanidine), reducing agents (e.g., dithiothreitol (DTT), beta mercaptoethanol, TCEP), urea, enzymes (e.g., ClpX, ClpS, unfoldases). Other biological or chemical agents may be included during the protein processing, e.g., lysozymes, papain, cruzain, trypsin, protease inhibitors, nucleases or nuclease-containing proteins (e.g., DNAse, RNAse, DNA glycosylases, restriction endonucleases, transposases, micrococcal nucleases, Cas proteins).

Peptides or proteins may be fragmented prior to analysis. Fragmenting proteins may be useful in reducing the size of the proteins and allow for efficient processing of peptides, as is described elsewhere herein. Fragmentation may be performed using proteases, e.g., trypsin, chymotrypsin, pepsin, Lys-C, Glu-C, Proteinase K, furin, thrombin, endopeptidase, papain, subtilisin, elastase, enterokinase, genenanse, endoproteinase, metalloproteases, or with chemical treatment, e.g., cyanogen bromide, hydrazine, hydroxylamine, formic acid, BNPS-skatole, iodosobenzoic acid, 2-nitro-5-thiocyanobenzoic acid, etc. Alternatively or in addition to, fragmentation may be performed using mechanical methods, such as sonication, vortexing, mechanical stirring, using temperature changes (e.g., freeze/thaw, heating), or other fragmentation approach.

Enrichment of proteins or peptides in a biological sample may be performed, e.g., for separating proteins and peptides from cellular debris or other types of analytes (e.g., nucleic acids, lipids, carbohydrates, metabolites). Such enrichment may include, for example, the use of affinity columns (e.g., ion exchange), size exclusion columns, affinity precipitation (e.g., immunoprecipitation), chromatography (e.g., HPLC), or electrophoresis. In instances where cells are partitioned prior to enrichment, the enrichment may be performed using microbeads, affinity microcolumns, affinity beads, etc. In some instances, fractionation may be performed on the proteins or peptides, which may be used to separate the proteins by size, hydrophobicity, charge, affinity, size, mass, density, etc.

Peptides may be barcoded, in bulk or in partitions. Peptides may be barcoded with any useful type of barcode molecule, e.g., spectral or fluorescent barcodes, mass tags, nucleic acid barcode molecules, etc. The barcode molecules may allow for identification of an originating peptide, a partition, a sample, a cell, or cell compartment. For example, a cell sample may be partitioned such that a partition comprises at most one cell; the partition may comprise a unique barcode molecule (e.g., nucleic acid barcode molecule) that identifies the partition and thus the cell. Subsequent labeling of the peptides within the partition (e.g., by permeabilizing or lysing the cell) with the barcode molecules may be useful in identifying the peptides as arising or originating from the same cell or partition. In other examples, a substrate may comprise nucleic acid molecules comprising a unique barcode sequence that differs from barcode sequences of other substrates. As such, the barcode sequence may be used to identify the substrate. In some instances, barcoded substrates may be partitioned with cell samples, such that at least a subset of the partitions comprise a single cell and a single barcoded substrate. As such, the peptides arising from the single cell and transferred to the barcoded substrate may all be identifiable as originating from the single cell. Barcode molecules may comprise additional useful functional sequences, e.g., UMIs, primer sites, restriction sites, cleavage sites, transposition sites, sequencing sites, read sites, etc.

Attachment of barcode molecules to peptides may be achieved using any suitable chemistry. For example, C-terminal conjugation of nucleic acid barcode molecules may be achieved by amide coupling of amine-conjugated DNA barcode molecules to peptides or by thiol alkylation, e.g., reacting a thiolated peptide with an alkylated (e.g., iodoacetamide) DNA barcode molecule. N-terminal conjugation can be achieved, for instance, using 2-pyridinecarboxyaldehyde labeling of a DNA barcode and reacting with the N-terminus of a peptide. Internal residues, e.g., glutamate, can also be labeled with amine-conjugated DNA barcode molecules or carboxylated DNA barcodes (e.g., to react with primary amines in lysine).

Individual peptides may be barcoded at multiple locations for a given peptide. A peptide may be labeled at multiple sites with the same or different barcode sequences. For example, a peptide may be partitioned into a partition comprising a plurality of identical barcode molecules that comprise a barcode sequence that is unique to the partition. The peptide may be labeled at a single or multiple sites with the unique partition barcode sequence, optionally each comprising a unique molecular identifier (UMI), such that subsequent downstream analysis (e.g., sequencing) may be attributable to the same peptide using the barcode sequence. In some instances, a terminus of the peptide (e.g., N-terminus or C-terminus) or an internal amino acid may be labeled with a barcode. In some instances, the peptide may be fragmented prior to analysis or sequencing; accordingly, upstream attachment of multiple identical barcode molecules to the same peptide may allow for attribution of the sequence analysis back to a single peptide. Barcoding of peptides may occur prior to, during, or subsequent to fragmentation. Peptides may be labeled with barcodes (e.g., nucleic acid barcode molecules) using any suitable chemistry, e.g., as described above, or using bifunctional or trifunctional linkers comprising multiple linking moieties, e.g., as described elsewhere herein, such as click chemistry moieties, NHS-esters, EDC, etc. For example, C-terminal attachment may comprise amide coupling to C-terminus carboxylic group or photoredox tagging of C-terminus carboxylic group (e.g., to add an electrophile tag). N-terminal attachment may comprise amide coupling to N-terminus amine group, where specific attachment can occur at low pH, or using 2-pyridinecarboxaldehyde variants for specific attachment to N-terminus. Internal attachment may comprise, for example, amide coupling using EDC/NHS chemistry or DMT-MM to Glutamate or Aspartate; alkylation or disulfide bridge labeling of cysteines; or amide coupling to lysine residues.

In some examples, a peptide may be labeled with different barcode molecules, which can be indexed by proximity to one another, e.g., using primers that can anneal to adjacent barcode molecules. In one such approach, after a protein has been labeled with a plurality of barcodes with different barcode sequences, proximity-based polymerase extension may be used to copy and associate the sequence of adjacent barcodes. For example, each barcode molecule may comprise a primer binding site, to which a dual-primer linker sequence comprising two sequences is annealed. The dual primer linker sequence can bind to the primer binding sites of two adjacent barcodes. An extension reaction, e.g., using a polymerase, may extend and copy the barcode sequences of the adjacent barcodes. Subsequently, the dual primer linker sequence, which now has copies of the two adjacent barcodes, may be removed and sequenced. From the sequencing reads, an adjacency matrix of barcode sequences may be generated (e.g., to correspond barcode sequences on a single dual primer linker as spatially adjacent). Accordingly, each of the barcode sequences may be associated with a nearby adjacent barcode sequences, and as such, peptide portions may be aligned or attributed as being adjacent. Such an approach may be useful in instances where the peptide is fragmented, such that individual fragments of a peptide may be corresponded with the nearest neighbor using the barcode sequences.

In another example, a peptide may be barcoded at multiple locations for a given peptide using bridge amplification. In such an approach, a peptide or protein may be labeled at multiple sites with a nucleic acid primer. A nucleic acid barcode molecule may be provided, which can anneal to the nucleic acid primer (not shown) or be ligated to the nucleic acid primer. Subsequent rounds of bridge amplification may be performed in order to copy the nucleic acid barcode molecule to the other primers located at other sites of the given peptide. In some examples, a peptide may be tagged with multiple copies of the nucleic acid primer, and barcode sequences may be provided sparsely, such that only one nucleic acid primer per peptide is extended by polymerase extension. Subsequent rounds of bridge amplification can result in a peptide having the same barcode sequence at each nucleic acid primer. Subsequent fragmenting of peptides may be performed, such that peptide fragments comprise on average, a single barcode. Accordingly, in some cases, the output such an amplification approach may be peptides with individual barcodes generated from fragmenting multi-labeled proteins where peptides from the same protein have the same barcodes.

A sample of cells may be partitioned into individual partitions or compartments (e.g., droplets, microwells) such that at least a subset of the partitions comprise a single cell. The partitions may then be treated with a lysing agent to lyse the cells and release the proteins from the cells into the partition. The proteins may then be labeled with a partition-specific barcode (e.g., using a barcode bead), such that all peptides or proteins arising from a single compartment comprises the same barcode. In some examples, the barcodes comprise nucleic acid barcode molecules, and the barcode sequence can be used in downstream processing, e.g., via sequencing, the partition or cell from which a peptide originated. The nucleic acid barcode molecule may comprise any additional useful sequences, e.g., UMIs, primer sequences, etc.

Bulk Processing: A biological sample may be processed in bulk. For example, a biological sample may be processed to obtain a suspension of cells, which may be directly lysed in the suspension, without partitioning of cells in individual compartments. Cells may be lysed in bulk using any useful approach, e.g., as described above and optionally subjected to further processing, e.g., homogenization, protease inhibition, denaturation, protein processing (e.g., chemical treatment, fragmentation), or a combination thereof A biological sample may be subjected to pre-processing prior to cell lysis or protein extraction. Such pre-processing may include removal of debris, purification, filtration, concentration, or sorting.

Spatial barcoding: A biological sample may comprise a tissue sample comprising multiple cells. Tissue samples may be processed using an approach to retain spatial information (e.g., to identify peptides from individual cells), e.g., using spatial barcodes. For instance, a 2-D or 3-D tissue sample may be provided, and individual cells or locations within a tissue sample may be contacted with a plurality of spatial barcodes (e.g., nucleic acid barcode molecules) comprising different barcode sequences. The different barcode sequences may be attributed to a particular location in the 2-D or 3-D tissue sample, which may correspond with a location of a cell. For example, spatial barcodes may be provided using deterministic methods such as two-photon patterning, or stochastic methods such as PCR, to assign different segments of the 2-D or 3-D tissue sample with unique spatial barcodes Accordingly, peptides that are labeled with spatial barcodes may be attributed back to a single location within a tissue sample, or back to a single cell.

Computer systems

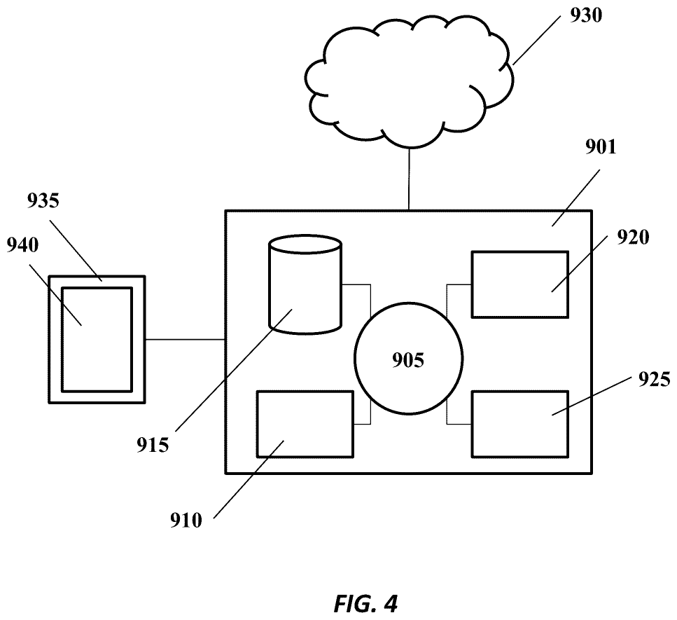

The present disclosure provides computer systems that are programmed to implement methods of the disclosure. FIG. 4 shows a computer system 401 that is programmed or otherwise configured to detect a detectable complex. In some embodiments, the computer system is programmed or otherwise configured to use a sequencing agent of Formula I (e.g., or I, IV, IV′, IV″, IV-A, IV-B, or V-C) to detect a detectable complex. In some embodiments, the detectable complex comprises an amino acid or derivative thereof. In some embodiments, the detectable complex comprises a peptide. In some embodiments, the computer system is programmed or otherwise configured to provide sequencing data of a polypeptide. The computer system 401 can regulate various aspects of detecting of the present disclosure, such as, for example, providing sequencing data of a polypeptide. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, sequencing data of a polymeric analyte. In some embodiments, the polymeric analyte comprises a peptide. In some embodiments, the sequencing data comprises sequencing reads from a polymerizable molecule, peptide identity, or both. In some embodiments, the sequencing data comprises peptide mass. In some embodiments, the polymeric analyte is a polypeptide. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example use the sequence data to provide an identity and order of each or a subset of amino acids of a peptide.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

### Example 1. Characterization and Validation of Sequencing Reagent

The sequencing reagents provided herein, such as sequencing reagents of Formula I, IV, IV′, IV″, IV-A, IV-B, or V-C, can conjugate to and cleave N-terminal amino acids and also comprise a capture-binding moiety that can enable conjugation of the sequencing reagent to a capture moiety (e.g., a peptide-bound capture moiety, a substrate-bound capture moiety). Cleavage and detection comprise conjugating the guanidinylating group (e.g., guanidination) to a peptide or terminal residue of an amino acid of known molecular weight and measuring the molecular weight before and after cleavage using mass spectrometry. In some instances, one or more products of the reaction(s) may be performed, e.g., using high performance liquid chromatography (HPLC). The expected reduction in molecular weight by loss of one amino acid will signify a successful cleavage. Mass spectrometry is performed on the peptide only without the sequencing reagent, peptide with the sequencing reagent conjugated to the N-terminal amino acid, and the cleaved peptide after the sequencing reagent removes the N-terminal amino acid. Alternatively, or in addition to, the conjugation reaction and the cleavage reaction can be monitored using liquid chromatography mass spectrometry (LCMS) for 30 minutes at 15 minute intervals for each reaction. In one such example protocol, conjugation of the sequencing reagent to the peptide can be performed using a reaction solvent containing the peptide in DMF, 2.5 equivalents of TEA or DIPEA at ambient temperature (20 degrees Celsius) for 30 minutes. The reaction is monitored using LCMS every 15 minutes for 30-60 minutes. The reaction is conducted under inert gas conditions. For the cleavage reaction, the purified product from the conjugation reaction is taken and 2% NaOH is added. The reaction is placed in a preheated oil or water bath at 37 degrees Celsius and the reaction is monitored every 15 minutes using LCMS and optionally NMR for 30-60 minutes. The cleavage reaction is conducted under inert gas.

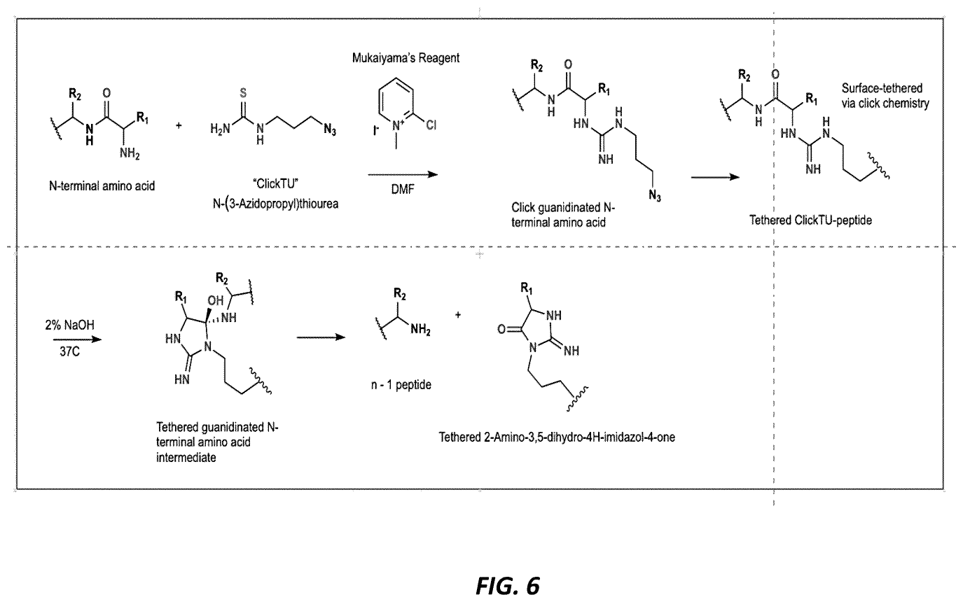

FIG. 5 and FIG. 6 show examples of mechanisms of cleavage of an amino acid using a thiourea, or click chemistry (e.g., azide) functionalized thiourea, to conjugate the guanidyl group. A peptide may be contacted with the thiourea (e.g., thiourea or N-(3-Azidopropyl)thiourea) in dimethylformamide (DMF) with the addition of Mukaiyama's reagent (2-chloro-1-methylpyridinium iodide). This reaction results in the formation of a guanidinylated N-terminal amino acid (sequencing reagent-monomer complex). In instances where the thiourea used was functionalized with a click-chemistry moiety, such as an azide, the guanidinylated peptide can be surface-tethered or coupled to a polymerizable molecule (e.g., nucleic acid molecule) through the click-chemistry moiety, such as through an azide/alkyne reaction. A heterocyclic guanidinylated N-terminal amino acid derivative can be formed by reaction of the guanidinylated peptide in 2% sodium hydroxide (NaOH) at 37° C. This intermediate can undergo cleavage to generate the terminal peptide (n-1 peptide) and optionally-tethered detectable complex 2-amino-3,5-dihydro-4H-imidazol-4-one. The tethered 2-amino-3,5-dihydro-4H-imidazol-4-one may be detected using any useful approach described herein, e.g., using a binding agent, using a nanopore sequencer, imaging or fluorescence detection, etc. or a combination thereof.

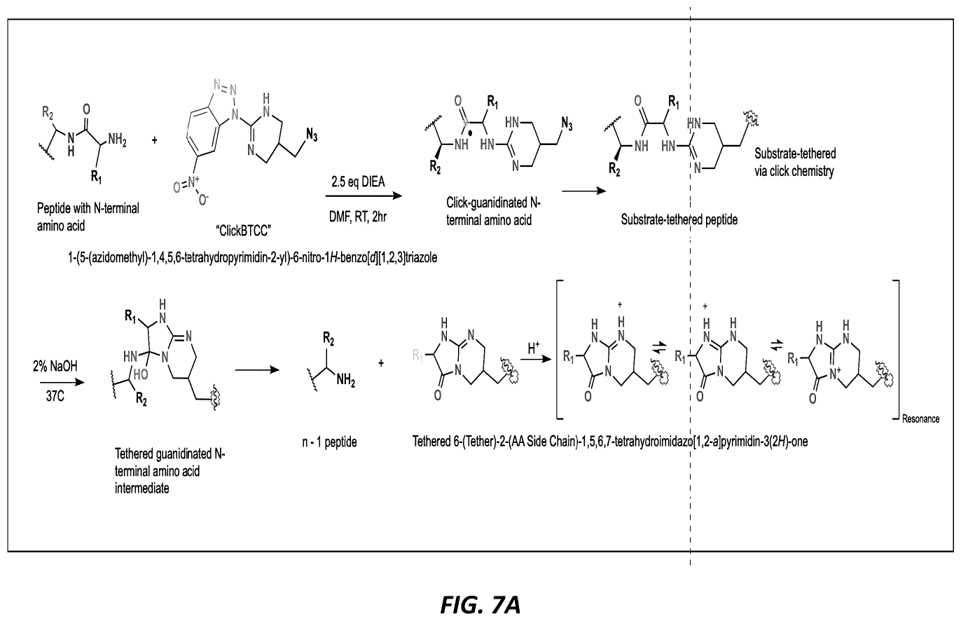

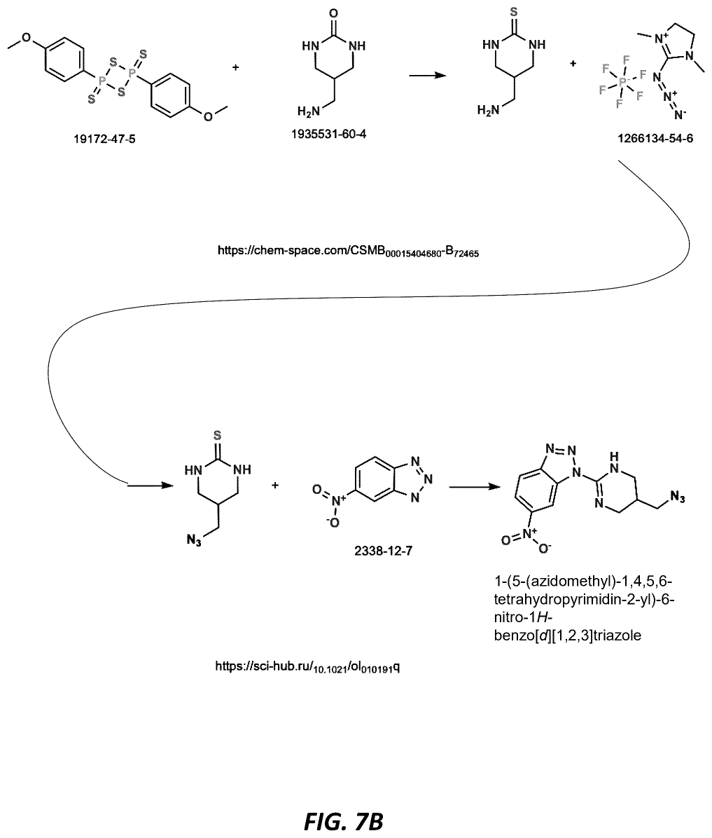

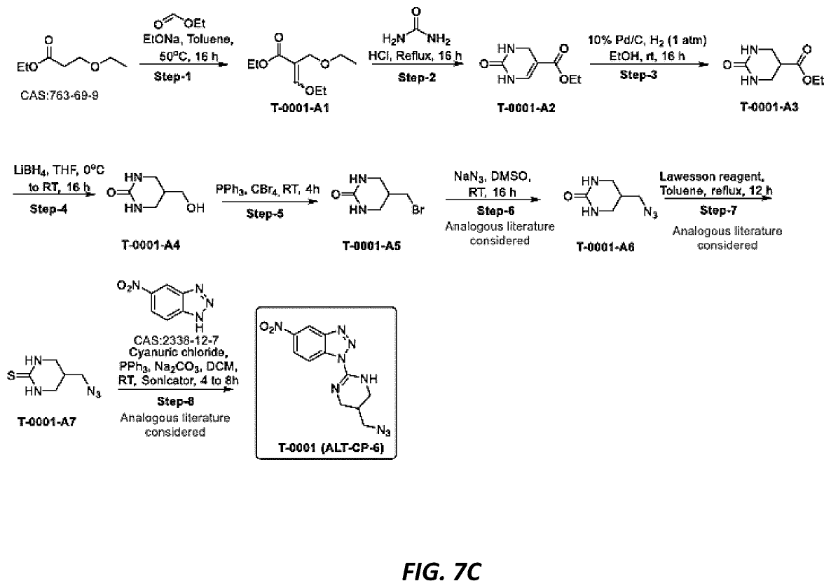

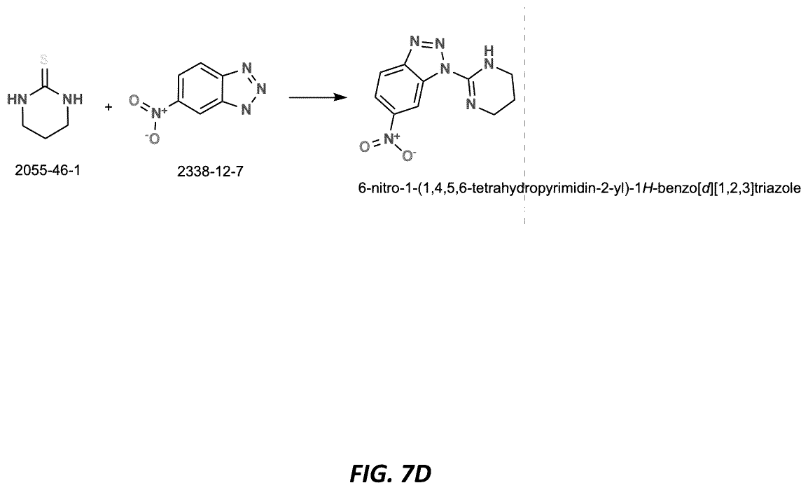

In other instances, the N-terminal amino acid may be contacted with a sequencing reagent that comprises a guanidinylating agent, such as 6-nitrobenzotriazole-1-carboxamidine, and a click chemistry (e.g., azide) moiety, illustrated in FIG. 7A. The sequencing reagent, 1-(5-(azidomethyl)-1,4,5,6,-tetrahydropyrimidin-2-yl)-6-nitro-1H-benzo[d][1,2,3]triazole, is contacted with the N-terminal amino acid of a peptide under conditions sufficient to guanidinylate the N-terminal amino acid (thereby generating a sequencing reagent-monomer complex). For example, the conjugation reaction may be performed using 2.5 equivalents of N,N-diisopropylethylamine in DMF for 2 hours at room temperature, which can result in the guanidinylated N-terminal amino acid (sequencing reagent-monomer complex). The resultant compound can be surface-tethered or coupled to a polymerizable molecule (e.g., a nucleic acid molecule) through the azide moiety, such as through an azide/alkyne click chemistry reaction. The guanidinylated N-terminal amino acid can then be cleaved from the peptide by reacting the compound in 2% NaOH at 37° C. to form a guanidinlyated N-terminal amino acid intermediate which can further react and undergo cleavage to form the remaining (cleaved)N-terminal amino acid (n-1 peptide) and a detectable complex comprising an imidazolone amino acid derivative (a derivative of the cleaved amino acid). The tethered, detectable complex may be detected, as described elsewhere herein.

FIG. 7B shows an example synthesis scheme for generating the sequencing reagent of FIG. 7A. FIG. 7C shows a synthesis scheme for a sequencing reagent similar to that shown in FIG. 7A, with the NO₂ moiety on a different carbon of the phenyl ring. FIG. 7D shows a synthesis scheme for a similar compound as FIG. 7A, 6-nitro-1-(1,4,5,6-tetrahydropyrimidin-2-yl)-1H-benzo[d][1,2,3]triazole, that comprises a guanidinylating agent but does not comprise an azide linker. The synthesis reaction is performed in DMF and triethylamine using mercury chloride (HgCl₂) at room temperature.

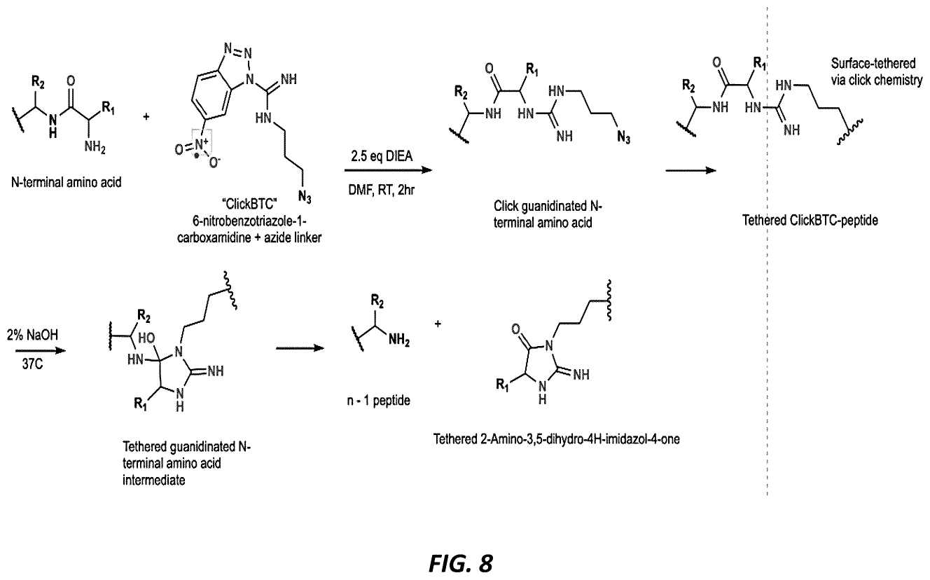

FIG. 8 shows another example sequencing reagent comprising a guanidinylating agent group, an alkyl linker, and a click chemistry (azide) moiety. Contacting of the N-terminal amino acid with the sequencing reagent, N-(3-azidopropyl)-6-nitro-1H-benzo[d][1,2,3]triazole-1-carboximidamide with the addition of 2.5 equivalents of N,N-diisopropylethylamine in DMF for 2 hours at room temperature results in the guanidinylated N-terminal amino acid (sequencing reagent-monomer complex), which can be surface-tethered or coupled to a polymerizable molecule through the azide, such as through an azide/alkyne click chemistry reaction. The guanidinylated N-terminal amino acid can be reacted in 2% NaOH at 37° C. to form a guanidinylated N-terminal amino acid intermediate which can further react to form the remaining (cleaved)N-terminal amino acid (n-1 peptide) and a substrate-tethered or polymerizable molecule-tethered detectable complex comprising 2-amino-3,5-dihydro-4H-imidazol-4-one (a derivative of the cleaved amino acid). The tethered, detectable complex may be detected, as described elsewhere herein.

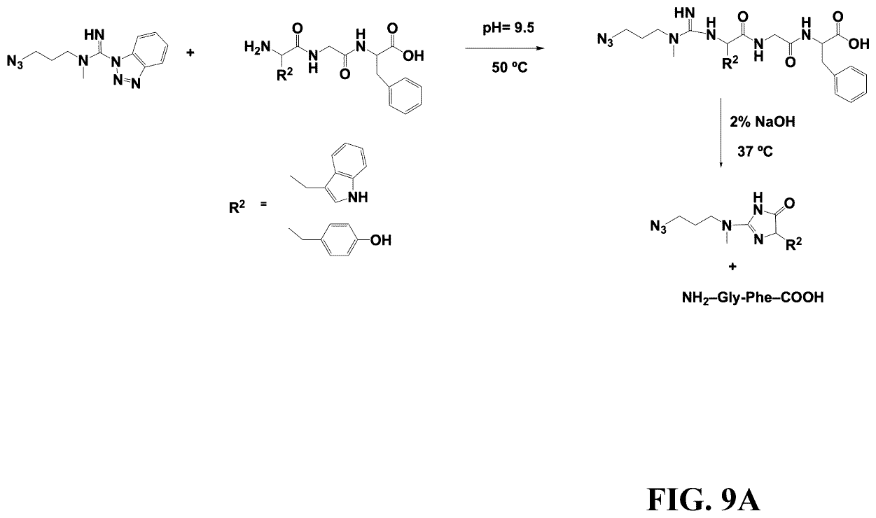

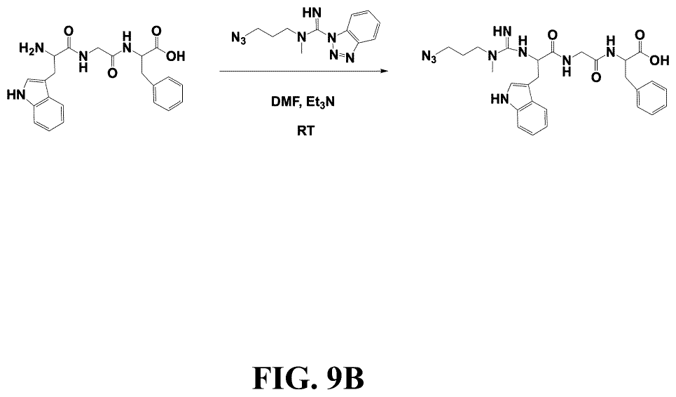

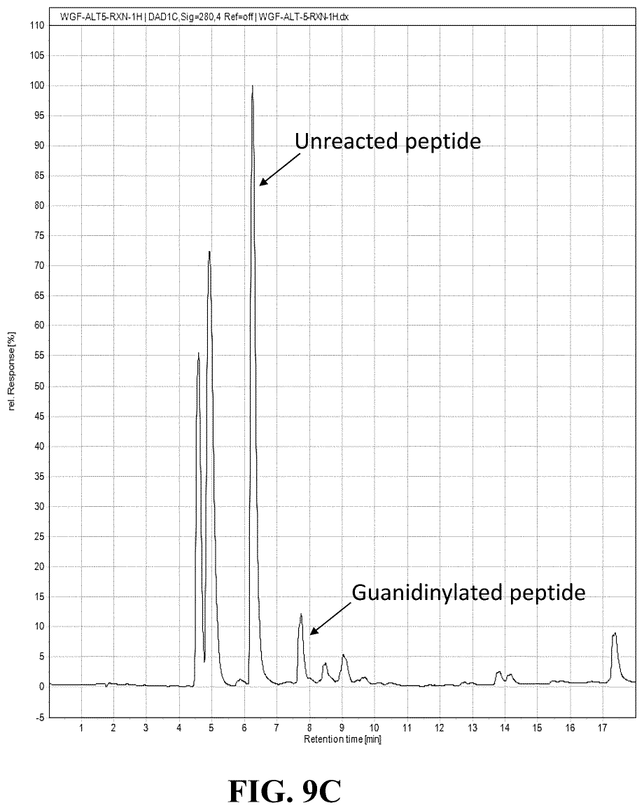

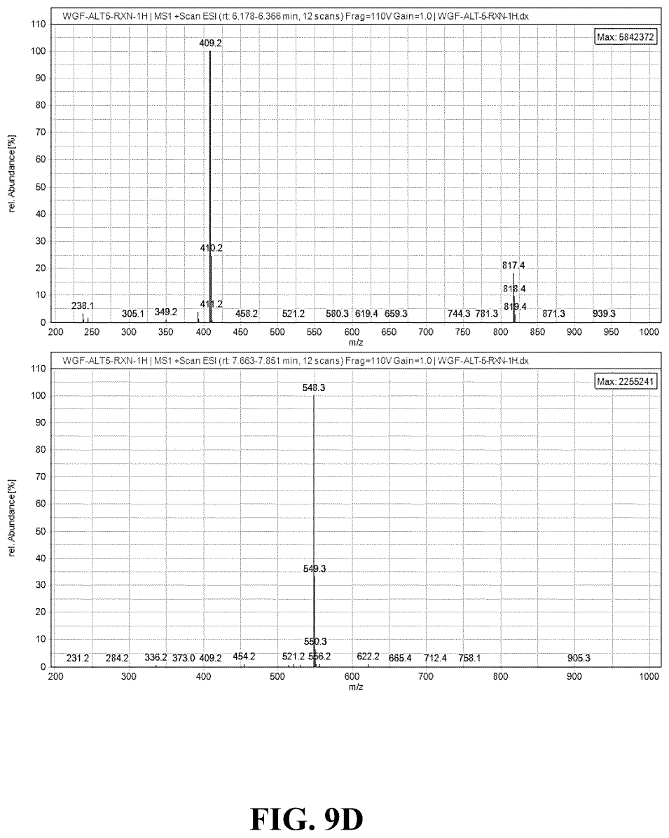

FIG. 9A shows another example sequencing reagent comprising a guanidinylating agent. Contacting of the N-terminal amino acid of a tripeptide with the sequencing reagent, N-(3-azidopropyl)-N-methyl-1 H-benzo[d][1,2,3]triazole-1-carboximidamide under mildly alkaline conditions (pH 9.5 at 50 degrees Celsius) results in the guanidinylated N-terminal amino acid (sequencing reagent-monomer complex), which can be surface-tethered or coupled to a polymerizable molecule through the azide moiety, such as through an azide/alkyne click chemistry reaction. The guanidinylated N-terminal amino acid can be reacted in 2% NaOH at 37° C. to form the remaining (cleaved)N-terminal amino acid (n-1 peptide) and a substrate-tethered or polymerizable molecule-tethered detectable complex. The tethered, detectable complex may be detected, as described elsewhere herein. It will be appreciated that while a particular sequencing reagent is shown in FIG. 9A, the reaction scheme can be generalized to other sequencing reagents, e.g., sequencing reagent of Formula I, IV, IV′, IV″, IV-A, IV-B, or V-C, and other peptides, e.g., different number of amino acids, different amino acid residues, etc.

FIG. 9B shows the same sequencing reagent as FIG. 9A using a different reaction condition for conjugation to the tripeptide (W-G-F). The sequencing reagent, N-(3-azidopropyl)-N-methyl-1 H-benzo[d][1,2,3]triazole-i-carboximidamide is reacted with a tripeptide at a 10:1 ratio using DMF and triethylamine at room temperature for 1 h, thereby generating a guanidinylated N-terminal amino acid (sequencing reagent-monomer complex), which can be surface-tethered or coupled to a polymerizable molecule through the azide moiety (not shown), such as through an azide/alkyne click chemistry reaction. The guanidinylated N-terminal amino acid can then be cleaved from the peptide using 2% NaOH at 37° C. to form the remaining (cleaved) N-terminal amino acid (n-1 peptide) and a substrate-tethered or polymerizable molecule-tethered detectable complex (not shown). The tethered, detectable complex may be detected, as described elsewhere herein.

FIGS. 9C-9D show LCMS data of the conjugation reaction shown in FIG. 9B. FIG. 9C shows the LC chromatogram. FIG. 9D shows the mass spectra for the peaks at ˜6.3 minutes and ˜7.7 minutes. The peak observed at ˜6.3 minutes indicates the unreacted tripeptide, and the peak at ˜7.7 minutes indicates the guanidinylated peptide (guanidinylated N-terminal amino acid, prior to cleavage of the N-terminal amino acid from the peptide).

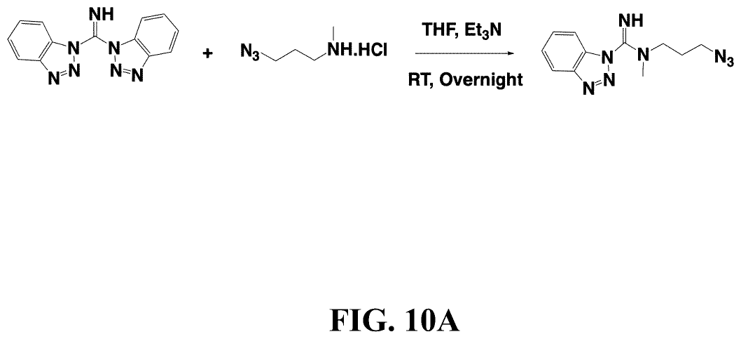

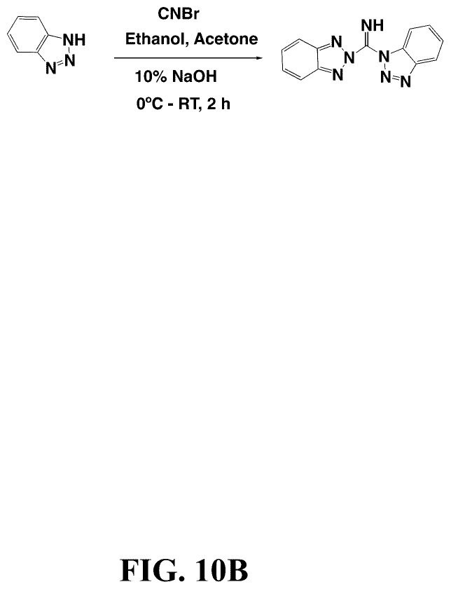

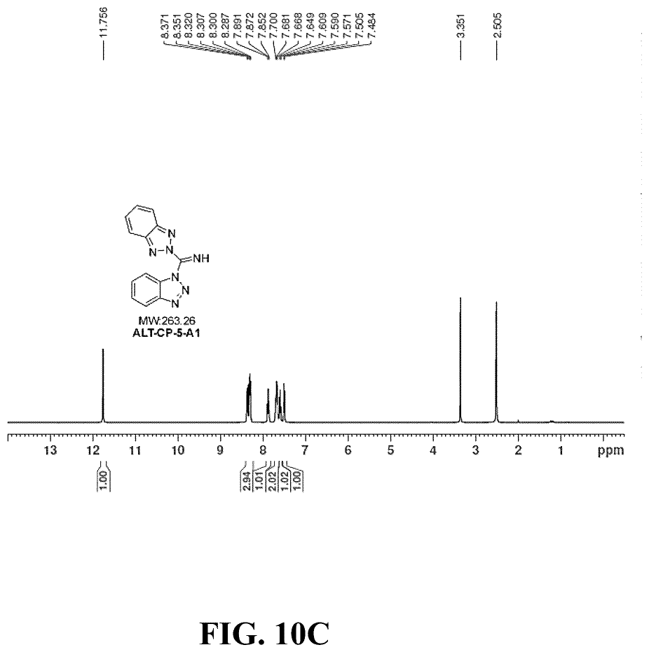

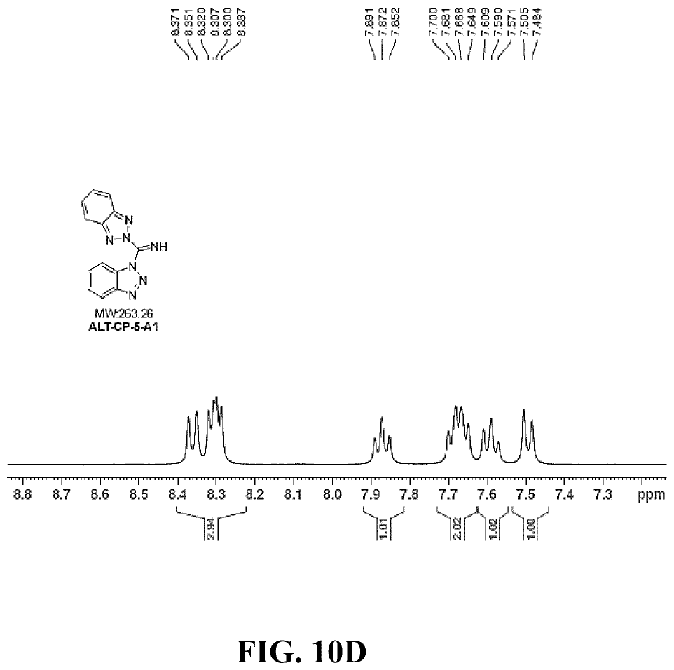

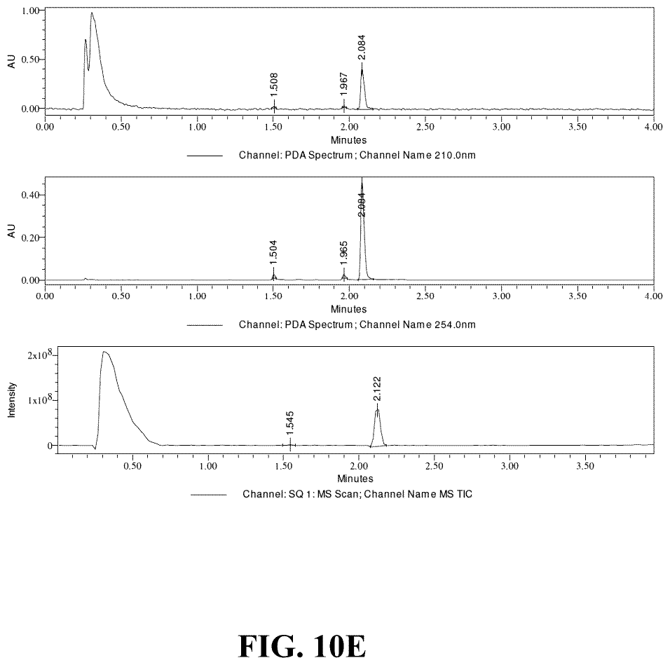

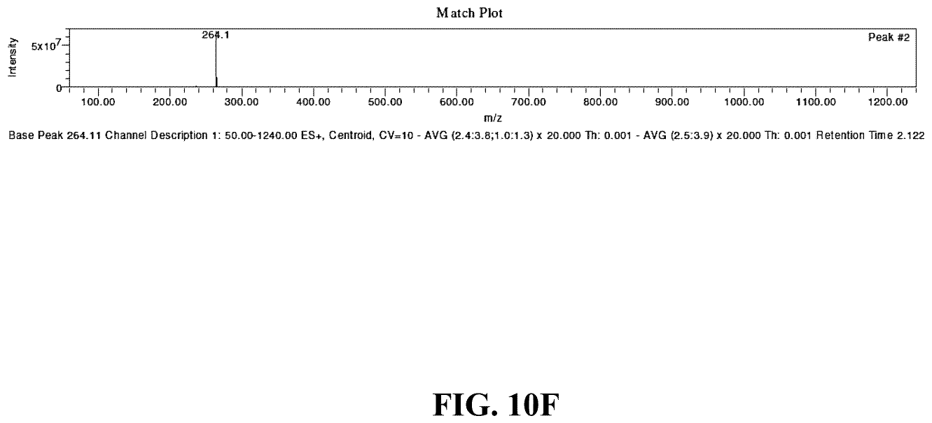

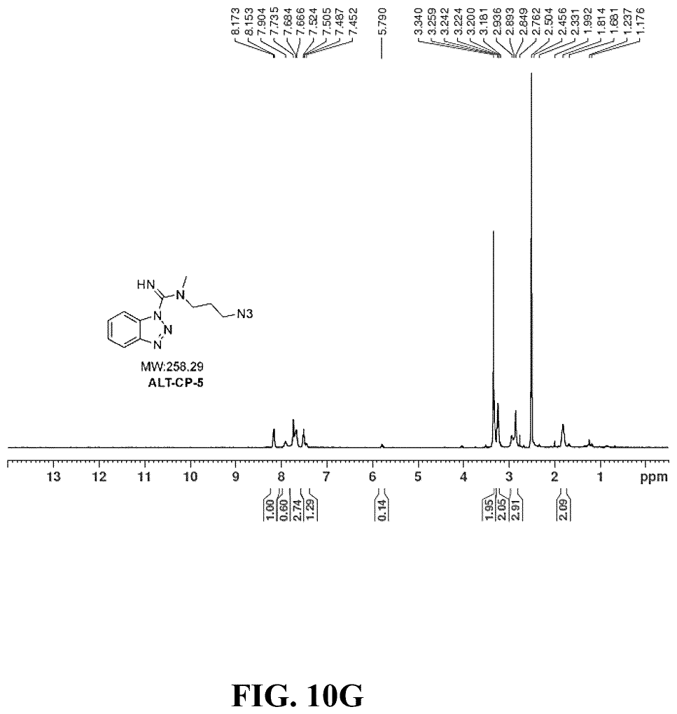

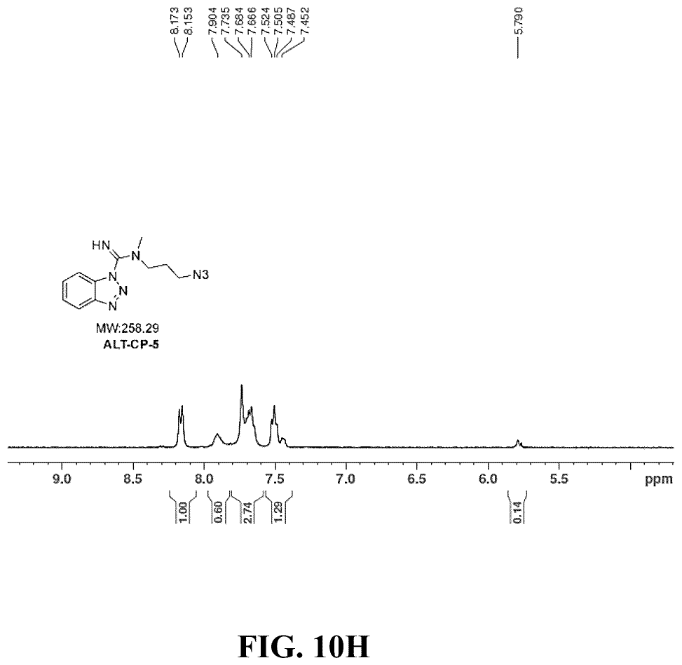

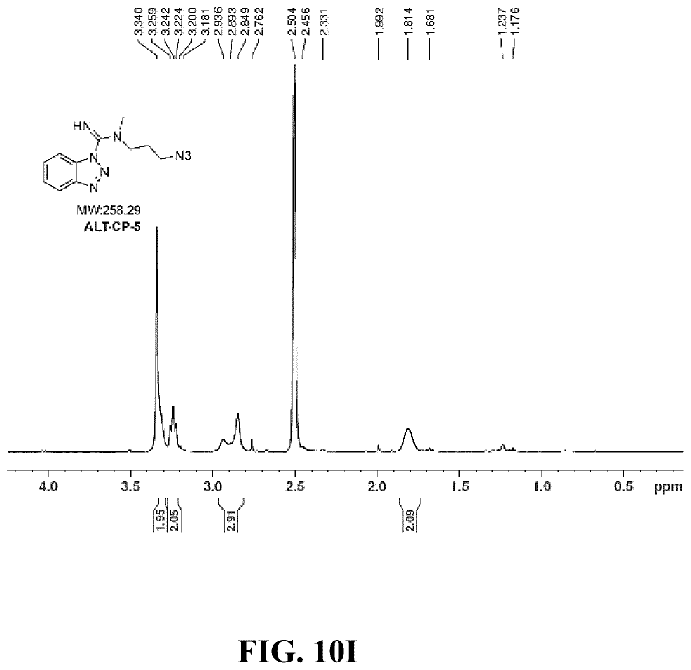

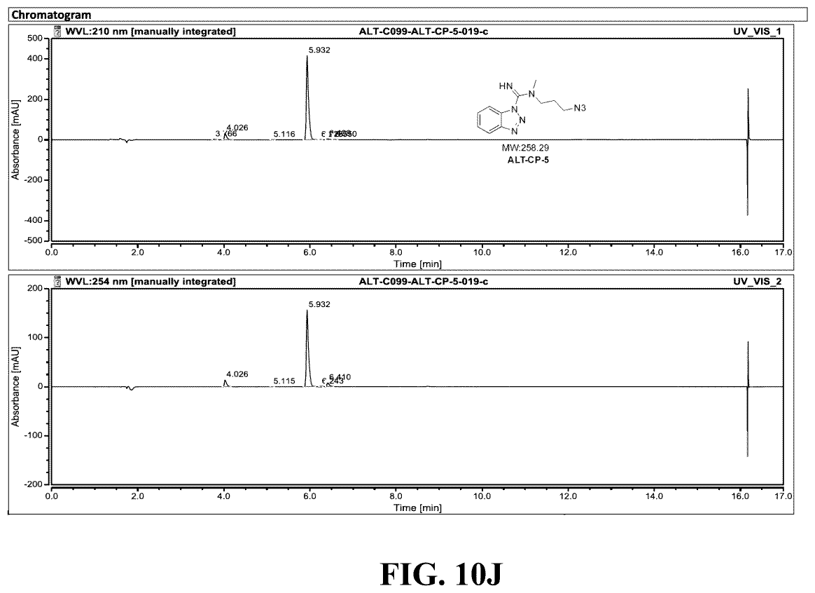

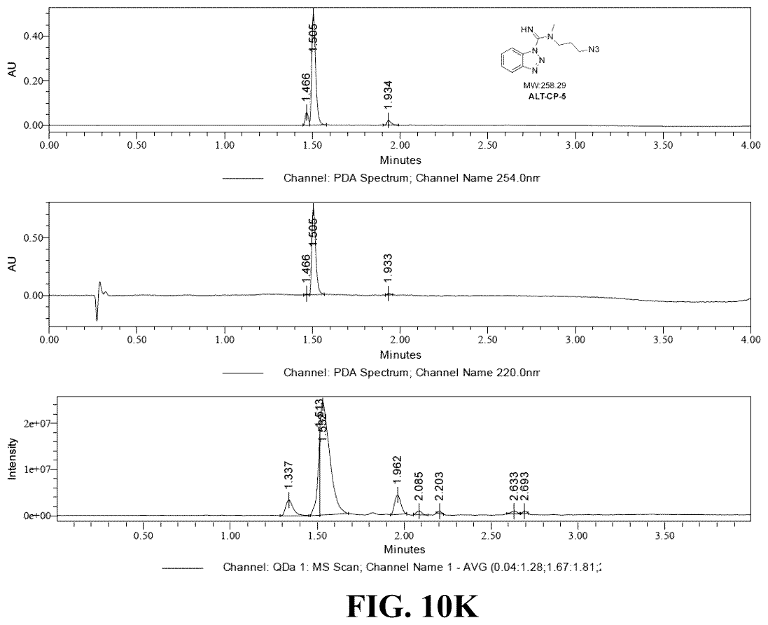

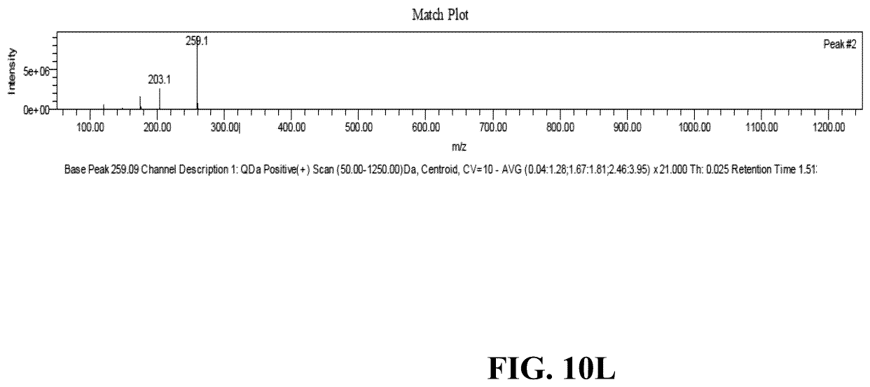

FIG. 10A shows an example synthesis scheme for generating the sequencing reagent shown in FIGS. 9A-9B. For the synthesis scheme of FIG. 10A, a stirred solution of (1H-benzo[d][1,2,3]triazol-1-yl)(2H-benzo[d][1,2,3]triazol-2-yl)methanimine (2.0 g, 7.66 mmol, 1.0 eq) in THF (20 mL) was added to triethyl amine (3.0 g, 30.64 mmol, 4.0 eq) at 0 degrees C. followed by addition of 3-azido-N-methylpropan-1-amine hydrochloride (2.16 g, 14.34 mmol, 1.9 eq) and stirred overnight at room temperature. After completion of the reaction as indicated by TLC (80% EtOAc in Hexane as a mobile phase), the reaction mixture was poured into ice-cold water (20 mL) and extracted with diethyl ether (3×100 mL). The combined organic layers were dried over Na₂SO₄ and concentrated under reduced pressure to afford the crude product. The obtained crude was purified by normal phase column chromatography using combi-flash with 230-400 silica as stationary phase and gradient 60% EtOAc in Hexane afforded the desired compound as a colorless liquid. (0.1 g, 5.10%). Analysis on the reaction was performed using LCMS and NMR.

FIG. 10B shows a synthesis scheme for generating (1H-benzo[d][1,2,3]triazol-1-yl)(2H-benzo[d][1,2,3]triazol-2-yl)methanimine), a precursor used for synthesis of the sequencing reagent shown in FIGS. 9A-9B. To a stirred solution of 1H-benzo[d][1,2,3]triazole (10.0 g, 94.41 mmol, 1.0 eq) in EtOH (450 mL) was added Cyanogen bromide (22.80 g, 191.65 mmol, 2.03 eq) in acetone (40 mL) at 0° C. The 10% NaOH solution (40 mL) was added in one portion and resulting mixture was stirred at same temperature for 2h (white precipitate observed). After, completion of the reaction as indicated by TLC (³0% EtOAc in Hexane as a mobile phase), the reaction mixture was filtered off and white precipitate was collected, washed with water (50 mL) and ethanol (50 mL). The precipitate was dried under vacuum to afford the title compound as white solid (5.5 g, 24.89%). Analysis on the reaction was performed using LCMS and NMR.

FIGS. 10C-10D shows NMR spectra of the resultant synthesis reaction for 1H-benzo[d][1,2,3]triazol-1-yl)(2H-benzo[d][1,2,3]triazol-2-yl)methanimine shown in FIG. 10B.

FIGS. 10E-10F show LCMS data of the resultant synthesis reaction for 1H-benzo[d][1,2,3]triazol-1-yl)(2H-benzo[d][1,2,3]triazol-2-yl)methanimine shown in FIG. 10B. The liquid chromatogram is shown in FIG. 10E, and the mass spectra are shown in FIG. 10F.

FIGS. 10G-10I show NMR spectra of the sequencing reagent of FIGS. 9A-9B, (N-(3-azidopropyl)-N-methyl-1 H-benzo[d][1,2,3]triazole-1-carboximidamide), which is synthesized using the synthesis scheme of FIG. 10A.

FIG. 10J shows an HPLC chromatogram of the sequencing reagent of FIGS. 9A-9B, (N-(3-azidopropyl)-N-methyl-1 H-benzo[d][1,2,3]triazole-1-carboximidamide), which is synthesized using the synthesis scheme of FIG. 10A.

FIGS. 10K-10L show LCMS data of the sequencing reagent of FIGS. 9A-9B, (N-(3-azidopropyl)-N-methyl-1 H-benzo[d][1,2,3]triazole-1-carboximidamide), which is synthesized using the synthesis scheme of FIG. 10A. The liquid chromatogram is shown in FIG. 10K, and the mass spectra are shown in FIG. 10L.

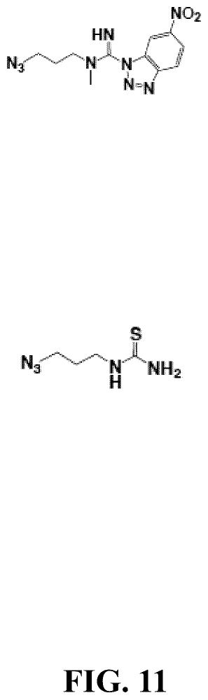

FIG. 11 shows two additional examples of sequencing reagents, N-(3-azidopropyl)-N-methyl-6-nitro-1H-benzo[d][1,2,3]triazole-1-carboximidamide (top) and 1-(3-azidopropyl)thiourea (bottom) that comprise a guanidinylating agent group and a click chemistry moiety (azide) that are connected by an alkyl linker (propyl).

FIG. 12 shows additional example guanidinylating agents that can be modified for use as a sequencing reagent described herein. For instance, a guanidinylating agent such as 1-amidinopyrazole hydrochloride, 1-carbamimidoyl-1,2,4-triazole hydrochloride, 1-(N-Boc-amidino)-pyrazole, or 1-(N-Cbz-amidino)-pyrazole can be modified to comprise a linker (e.g., a methyl, ethyl, propyl, PEG, or other polymeric linker) and a capture-binding moiety, e.g., click chemistry moiety, thereby generating a sequencing reagent.

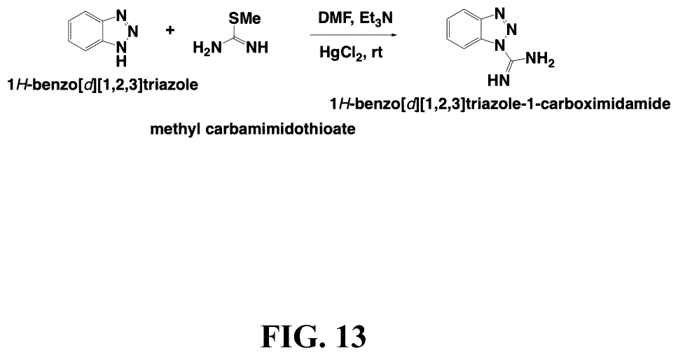

FIG. 13 shows a synthesis scheme for generating a guanidinylating agent that can be further modified for use as a sequencing reagent. For instance, a guanidinylating agent such as 1H-benzo[d][1,2,3]triazole-1-carboximidamide can be synthesized using commercially available reagents 1H-benzo[d][1,2,3]triazole and methyl carbamimidothioate and reacting the compounds in DMF and triethylamine with mercury chloride (HgCl₂). The 1H-benzo[d][1,2,3]triazole-1-carboximidamide can be further modified (not shown) to comprise a linker (e.g., a methyl, ethyl, propyl, PEG, or other polymeric linker) and a capture-binding moiety, e.g., click chemistry moiety, thereby generating a sequencing reagent.

The functionality of the azide group as the capture-binding moiety and its ability to conjugate to alkyne-substrates through click chemistry is determined. To demonstrate this, functionalized beads are either coated without peptide (control) or with immobilized peptides (e.g., using a linker, as described elsewhere herein). Following coating, beads are incubated with any of the sequencing reagents provided herein, such as a sequencing reagent of any of Formula I-IV, to facilitate conjugation (e.g., guanidination) to the N-terminal amino acid of the peptides. Next, the clickable group (e.g., azide) of the sequencing reagent is reacted with an alkyne (e.g., DBCO) connected to a fluorophore, such as tetramethylrhodamine (TMR), followed by a washing step, which can remove unreacted fluorophore. Since the reactive alkyne-fluorophore should only react with the azide group of the sequencing reagent, the sequencing reagent conjugated to the peptide containing beads should be higher in fluorescent intensity when the alkyne-fluorophore is introduced. The increase in fluorescence when compared to the control will be indicative of a functional azide-alkyne click chemistry on the sequencing reagent.

The sequencing reagent may comprise a cleavable linker, which can enable removal or cleavage of at least a portion of the sequencing reagent from a substrate. In some embodiments, the cleavable linker comprises a disulfide bond, which may be cleaved upon addition of a reducing agent (e.g., dithiothreitol or TCEP). In one example, a sequencing reagent comprising a modular thiol-azide linker will be tested to ensure that it forms a disulfide bond with thiol-functionalized surfaces for conditionally immobilizing the sequencing reagent. This validation will involve using the linker to form disulfide bonds on thiol-functionalized beads and using the azide group on the linker to conjugate to an alkyne-fluorophore conjugate. Adding the reducing agent TCEP is expected to cleave the disulfide bond and release the linker conjugated to the fluorophore, thus reducing the fluorescent intensity. Controls will test whether disulfide bonds under amino acid cleavage conditions will cleave and release the fluorophore and also whether the fluorophore itself is stable when exposed to TCEP and amino acid cleavage conditions. The fluorophore will be directly conjugated to beads and expected to maintain the same fluorescent intensity when under amino acid cleavage conditions (e.g., NaOH solution) and exposed to the reducing agent TCEP.

In some embodiments, the sequencing reagent comprises a polymerizable molecule that comprises a capture-binding moiety (e.g., nucleic acid sequence that can couple to a substrate-bound nucleic acid molecule or to a capture moiety coupled to a polymeric analyte such as a peptide). The sequencing reagent may be generated, for example, using a sequencing reagent precursor that comprises a guanidinylating agent (amino acid reactive group) and a first reactive moiety (e.g., a click chemistry moiety) and reacting the sequencing reagent precursor with a polymerizable molecule comprising a second reactive moiety (e.g., a click chemistry moiety). The first reactive moiety and the second reactive moiety may react (e.g., via strain promoted azide-alkyne cycloaddition or other click chemistry reaction) to covalently link the polymerizable molecule to the substate-tethering moiety. Accordingly, the sequencing reagent may comprise Formula I (e.g., or IV, IV′, IV″, IV-A, IV-B, or V-C), in which A is a guanidinylating agent, Li comprises a linker, and B comprises the polymerizable molecule (e.g., nucleic acid molecule).

### Example 2. Imaging and Scaling to Proteome

Amino acids will be identified by integrating all components of the sequencing reagent, isolation of the N-terminal amino acids, labeling the amino acids with the sequencing reagent-amino acid specific binding agents, detection (e.g., via imaging of fluorophores coupled to the binding agents, or via coupling of polymerizable molecules attached to the binding agents and substrate and sequencing the polymerizable molecules, as described elsewhere herein), and release of the sequencing reagent for subsequent cycles of amino acid identification. Alternatively, amino acids will be identified by integrating all components of the sequencing reagent, isolation of the N-terminal amino acids, optional iteration, and detection, e.g., using a nanopore sequencer to output the identity of the individual amino acids of a cleaved amino acid-linker complex. Sufficient cycles of amino acid identification will provide protein sequencing information.

Edman degradation and phenylisothiocyanate (PITC)-based reagents are typically used for peptide sequencing. However, alternative guanidination agents can provide several advantages over the traditional Edman reagents. For example, sequencing using guanidination agents requires milder reaction conditions, such as avoiding the need for high temperature and acidic conditions which are characteristic of Edman degradation. In some instances, guanidination requires the use of neutral or basic conditions, such as 2% NaOH. Additionally, guanidination agents can exhibit high efficiency and can exhibit faster kinetics than that of Edman reagents. In some instances, guanidination can result in isomer formation from alternative cyclization of the guanidine group. The generic mechanism of amino acid isolation using a guanidination agent can be seen below:

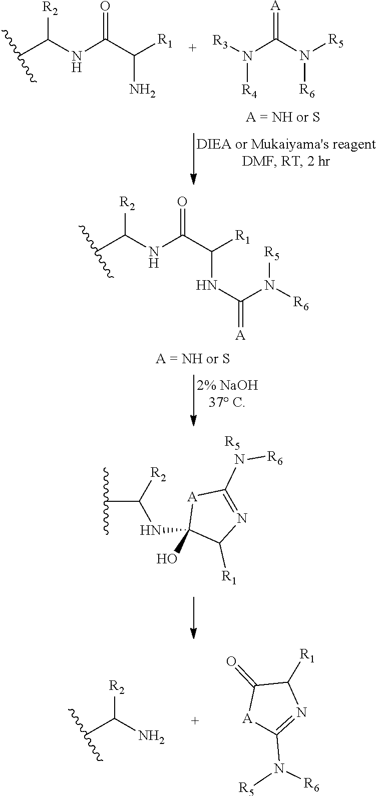

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-76. (canceled)

77. A method of using a sequencing reagent, comprising:
a. providing a capture moiety and a polymeric analyte;
b. contacting the polymeric analyte with the sequencing reagent, wherein the sequencing reagent comprises Formula I:
(Formula I), 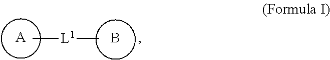
or a stereoisomer, tautomer, or salt thereof, wherein:
A comprises a reactive group configured to form a covalent bond with an N-terminal amino acid of a peptide, wherein the reactive group comprises a guanidinylating agent;
B comprises a capture-binding moiety; and
L¹ comprises a linker coupled to A and B,
wherein the sequencing reagent binds to a monomer of the polymeric analyte to form a sequencing reagent-monomer complex;
c. coupling the capture-binding moiety to the capture moiety; and
d. cleaving the sequencing reagent-monomer complex from the polymeric analyte, thereby providing a detectable complex.

78. The method of claim 77, further comprising:
e. detecting the detectable complex.

79. The method of claim 77, wherein the reactive group comprises a structure of Formula I-B: 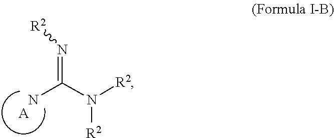
or a stereoisomer, tautomer, or salt thereof, wherein:
R² are each independently hydrogen, R⁴, OH, OR⁴, NH₂, or —NHR⁴;
R⁴ is C₁₋₆ alkyl or C₁₋₆ alkoxy, each of which is optionally substituted with halo, C₁₋₆ alkyl, C₁₋₆ alkoxy, C₁₋₆ haloalkyl, oxo, aryl, and 5-6-membered heteroaryl, wherein the aryl and heteroaryl are optionally substituted with one or two members selected from halo, —OH, C₁₋₄ alkyl, C₁₋₄ alkoxy, C₁₋₄ haloalkyl, NO₂, CN, COOR″, and CON(R″)₂, wherein each R″ is independently H or C₁₋₃ alkyl;
ring A is a 5-6-membered heteroaryl ring comprising up to three N atoms as ring members and is optionally fused to an additional aryl or 5-6-membered heteroaryl ring, and wherein the aryl and 5-6-membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R^{a}, —NR₂, phenyl, and 5-6 membered heteroaryl;
wherein each R is independently selected from hydrogen and C₁₋₃ alkyl optionally substituted with OH, OR^{a}, —NH₂, —NHR^{a}, or —NR^{a}₂; and
each R^{a} is C₁₋₃ alkyl, optionally substituted with OH, oxo, C₁₋₂ alkoxy, or CN;
wherein two R, or two R″, or two R^{a} on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, and CN.

80. The method of claim 79, wherein Ring A is pyrazole or triazole, each optionally fused with aryl.

81. The method of claim 79 wherein the reactive group comprising Formula I-B is: 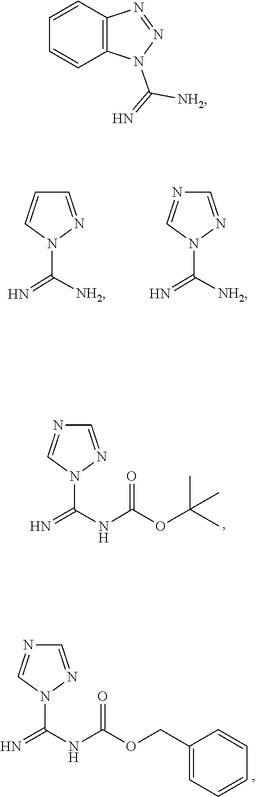 [SMILES: N=C(N)n1nnc2ccccc21 ; N=C(N)n1cccn1 ; N=C(N)n1cncn1 ; CC(C)(C)OC(=O)NC(=N)n1cncn1 ; N=C(NC(=O)OCc1ccccc1)n1cncn1]
stereoisomer, tautomer, or salt thereof.

82. The method of claim 77, wherein the capture-binding moiety comprises a polymer.

83. The sequencing reagent of claim 82, wherein the polymer comprises polyethylene glycol (PEG), deoxyribonucleic acid (DNA), or ribonucleic acid (RNA).

84. The method of claim 82, wherein the polymer is covalently linked to a substrate.

85. The method of claim 77, wherein the capture-binding moiety comprises a click chemistry moiety.

86. The method of claim 77, wherein the capture-binding moiety is configured to couple to a capture-moiety, wherein the capture moiety comprises a thiol group.

87. The method of claim 77, wherein the sequencing reagent comprises: 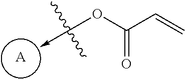
wherein 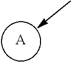
indicates orientation of the capture-binding moiety relative to the reactive group.

88. The method of claim 87, wherein the capture-binding moiety comprises a thiol group, and wherein the capture-binding moiety is configured to couple to a surface that comprises an acrylate group.

89. The method of claim 77, wherein the reactive group comprises Formula II-A: 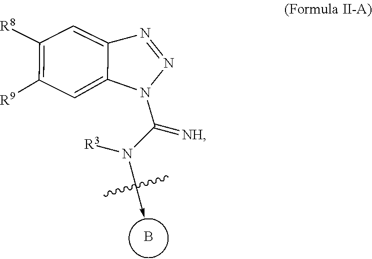
wherein:
R³ is hydrogen or substituted or unsubstituted C₁₋₆ alkyl, or heteroalkyl;
R⁸ and R⁹ are independently hydrogen or NO₂; and
wherein 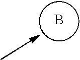
indicates orientation of the reactive group relative to the capture-binding moiety.

90. The method of claim 77, wherein L¹ comprises a cleavable linker, wherein the cleavable linker comprises a disulfide bond, a hydrazone, a PEG linker, a DNA molecule comprising a cleavage site, a peptide that is cleavable by an enzyme, an ester, or a de-click chemistry moiety.

91. The method of claim 77, wherein the reactive group is linked by a covalent bond with the N-terminal amino acid of the peptide.

92. The method of claim 77, wherein the sequencing reagent has a structure of Formula IV: 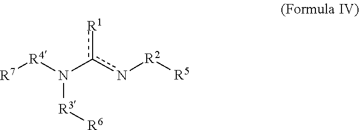
or a stereoisomer, tautomer, or salt thereof, wherein:
R¹ comprises one or more leaving groups;
R² and R^{3′} are independently selected from one or more linkers, hydrogen, or absent; or
R² and R^{3′} are taken together with the atoms from which they are attached to form an heteroaryl or heterocycloalkyl, the heteroaryl or heterocycloalkyl being optionally substituted with one or more linker-click-chemistry moieties;
R^{4′} is one or more linkers, hydrogen, or absent;
R⁵, R⁶, and R⁷ are independently click-chemistry moieties or absent; [symbol]
is a single or double bond;
wherein at least one of R⁵, R⁶, and R⁷ comprise a click-chemistry moiety.

93. The method of claim 92, wherein R² and R^{3′} are taken together with the atoms from which they are attached to form heterocycloalkyl substituted with one or more linker-click chemistry moieties.

94. The method of claim 92, wherein the leaving group comprises an electrophilic group.

95. The method of claim 94, wherein the electrophilic group comprises S, SCH₃, SO₃H, SO₂CF₃, or NHTf.

96. The method of claim 94, wherein the electrophilic group comprises SR*, wherein R* comprises hydrogen, R′, OH, OR′, NH₂, or NHR′, wherein R′ is a C₁₋₆ alkyl optionally substituted with one or more members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂, where each R″ is independently H or C₁₋₃ alkyl.

97. The method of claim 92, wherein the sequencing reagent has the structure of Formula IV-A: 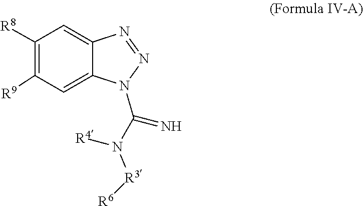
or a stereoisomer, tautomer, or salt thereof, wherein:
R^{4′} is substituted or unsubstituted C₁₋₆ alkyl, substituted or unsubstituted C₁₋₆ heteroalkyl, substituted or unsubstituted C₁₋₆ alkoxy, hydrogen, or absent;
R¹ and R⁹ are independently hydrogen or an electron withdrawing group.

98. The method of claim 97, wherein the electron withdrawing group comprises a haloalkyl, a halogen, an amide, a carbonyl, or NO₂.

99. The method of claim 77, wherein the sequencing reagent has a structure of Formula IV-B: 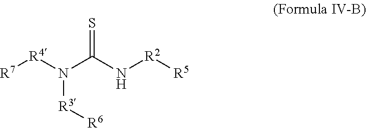
wherein:
R², R^{3′}, and R^{4′} are independently selected from hydrogen, one or more linkers, or absent; and
R⁵, R⁶, and R⁷ are independently click-chemistry moieties or absent.

100. The method of claim 77, wherein the sequencing reagent is: 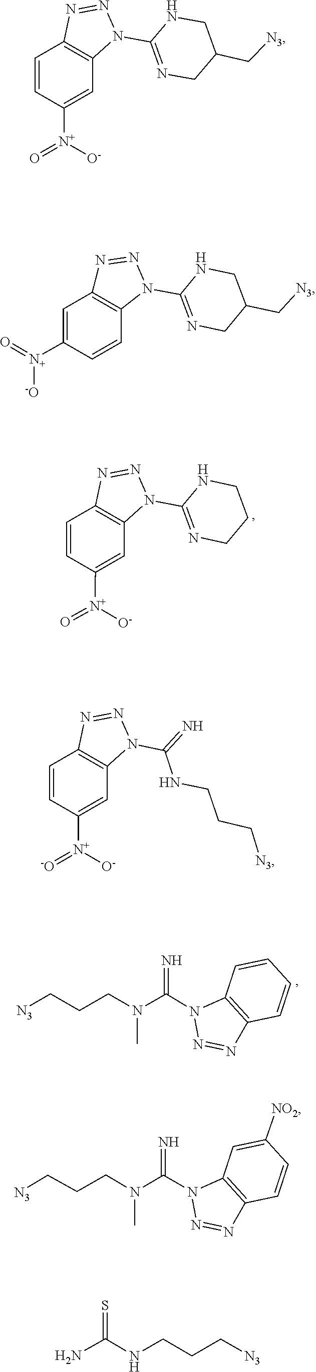
or a stereoisomer, tautomer, or salt thereof.

101. The method of claim 77, wherein the polymeric analyte comprises a polypeptide.

102. The method of claim 77, wherein the monomer comprises a terminal amino acid residue.

103. The method of claim 77, wherein the capture moiety comprises a DNA molecule.

104. The method of claim 77, wherein, in (a), the polymeric analyte is coupled to a substrate.

105. The method of claim 77, wherein (d) is performed chemically or enzymatically.

106. The method of claim 78, wherein (e) is performed using a nanopore sequencer.
